(19) United States
(12) Patent Application Publication
(10) Pub. No.: US 2025/0140348 A1
(43) Pub. Date: May 1, 2025

(54) METHODS AND SYSTEMS FOR PREDICTING AN ORIGIN OF AN ALTERATION IN A SAMPLE USING A STATISTICAL MODEL

(71) Applicant: Foundation Medicine, Inc., Boston, MA (US)

(72) Inventors: Alexander D. FINE, Cambridge, MA (US); Brennan DECKER, Winchester, MA (US); Zheng KUANG, Boston, MA (US); Chang XU, Germantown, MD (US); Daokun SUN, Cambridge, MA (US); Yanmei HUANG, Brookline, MA (US)

(73) Assignee: Foundation Medicine, Inc., Boston, MA (US)

(21) Appl. No.: 18/941,622

(22) Filed: Nov. 8, 2024

Related U.S. Application Data
(63) Continuation of application No. PCT/US2023/021754, filed on May 10, 2023.
(60) Provisional application No. 63/340,785, filed on May 11, 2022.

(51) Int. Cl. G16B 30/00; C12Q 1/6869; G16B 40/00

(52) U.S. Cl. CPC G16B 30/00; C12Q 1/6869; G16B 40/00

(57) ABSTRACT

Methods for predicting an origin of an alteration of a sample are described. The methods may comprise, for example, receiving, using one or more processors, sequence read data associated with the sample, selecting, using the one or more processors, a plurality of reads from the sequence read data based on the alteration, determining, using the one or more processors, at least one feature characterizing the selected plurality of reads, inputting, using the one or more processors, the at least one feature into a statistical model, generating, using the one or more processors, a score indicative of the origin of the alteration by the statistical model, and predicting, using the one or more processors, the origin of the alteration in the sample by comparing the score and one or more predefined thresholds.

### CROSS REFERENCE TO RELATED APPLICATIONS

This application is a continuation application of International Application No. PCT/US2023/021754, filed internationally on May 10, 2023, which claims the priority benefit of U.S. Provisional Patent Application Ser. No. 63/340,785, filed May 11, 2022, the disclosure of each of which is hereby incorporated by reference in its entirety.

### FIELD OF DISCLOSURE

The present disclosure relates generally to methods and systems for analyzing genomic profiling data, and more specifically to methods and systems for determining an origin of an alteration in a sample.

### BACKGROUND

Cell free DNA (cfDNA) testing can detect multiple categories of variants or alterations, including germline alleles, somatic mutations from tumor cells, and alterations that drive clonal hematopoiesis (CH). Due to overlap in the underlying biological processes, CH-derived alterations (e.g., CH alterations) can be misconstrued as tumor-derived alterations, which may confound clinical interpretation of liquid biopsy results. For example, CH alterations and tumor-derived alterations may have similar allelic fractions in cfDNA and can occur in the same genes. These CH alterations detected in a cfDNA test can change the medical interpretation of a liquid biopsy by, for example, falsely listing CH alterations as actionable tumor alterations, inflating estimations of tumor fraction, and impacting the calculation of many biomarkers.

An accurate predictor that distinguishes CH-derived alterations from tumor-derived alterations would improve the accuracy of cfDNA testing by identifying alterations derived from CH that are unlikely to predict treatment response. For example, if a healthcare provider recommends a therapy based on cfDNA test results that misidentify CH-derived alterations as tumor-derived, the treatment prescribed to the patient would likely be ineffective and would expose the patient to the potential risks of the therapy without the therapeutic benefits. Healthcare providers would thus benefit from having access to more accurate cfDNA testing that accounts for the presence of CH-derived alterations in a cfDNA sample to improve clinical management for the patient's solid tumor.

### BRIEF SUMMARY

Disclosed herein are methods and systems for determining an origin of an alteration in a sample. Also disclosed are methods for monitoring disease progression (e.g., cancer) in a patient. The disclosed methods have the potential to improve healthcare outcomes for patients (e.g., cancer patients) by providing healthcare providers with a test that can more accurately identify the presence of tumor-derived alterations in liquid biopsy samples by accounting for the presence of CH-derived alterations in the sample. Embodiments in accordance with this disclosure can further improve the testing and monitoring of patients by ensuring that patient samples include a sufficient amount of tumor-derived variations to make a clinical determination. Embodiments in accordance with this disclosure can further improve the testing and monitoring of patients by improving the detection of biomarkers based on the patient sample.

Described herein are systems and methods of prediction an origin of an alteration in a sample. An exemplary method of predicting an origin of an alteration in a sample comprises obtaining a first set of one or more samples from a subject, isolating polynucleotides from the first set of one or more samples, sequencing the isolated polynucleotides to produce sequence reads, selecting a plurality of reads from the sequence read data based on an alteration in the first set of one or more samples, determining at least one feature characterizing each of the selected plurality of reads, inputting the at least one feature characterizing each of the selected plurality of reads into a trained machine learning model, generating a score indicative of an origin of the alteration by the trained machine learning model, and predicting the origin of the alteration in the sample by comparing the score and one or more predefined thresholds.

In some embodiments, the alteration includes at least one of an insertion, a deletion, or a substitution. In some embodiments, the method can further include providing a plurality of nucleic acid molecules obtained from a sample from a subject, ligating one or more adapters onto one or more nucleic acid molecules from the plurality of nucleic acid molecules, amplifying the one or more ligated nucleic acid molecules from the plurality of nucleic acid molecules, capturing amplified nucleic acid molecules from the amplified nucleic acid molecules, sequencing, by a sequencer, the captured nucleic acid molecules to obtain a plurality of sequence reads that represent the captured nucleic acid molecules, receiving, at one or more processors, sequence read data for the plurality of sequence reads, receiving, using one or more processors, sequence read data associated with the sample, selecting, using the one or more processors, a plurality of reads from the sequence read data based on the alteration, determining, using the one or more processors, at least one feature characterizing the selected plurality of reads, inputting, using the one or more processors, the at least one feature into a statistical model, generating, using the one or more processors, a score indicative of the origin of the alteration by the statistical model, and predicting, using the one or more processors, the origin of the alteration in the sample by comparing the score and one or more predefined thresholds.

In some embodiments, the statistical model is a trained statistical model or an untrained statistical model. In some embodiments, the statistical model is a trained machine learning model and trained by: receiving, using the one or more processors, training data including information quantifying features related to the alteration; and training, using the one or more processors, the machine learning model based on the training data.

In some embodiments, the score is indicative of a probability that the alteration is derived from a solid tumor. In some embodiments, the score is further indicative of a probability that the alteration is derived from clonal hematopoiesis.

In some of embodiments, the at least one feature comprises at least one fragmentomic characteristic of the sample. In some embodiments, the at least one fragmentomic characteristic of the sample comprises at least one of: an amount of the selected plurality of reads having a specified length, a mean fragment length of the selected plurality of reads, a median fragment length of the selected plurality of reads, an interquartile range of fragment lengths of the selected plurality of reads, a distribution of fragment lengths of the selected plurality of reads, one or more peaks of fragment lengths for the selected plurality of reads, a fragment length for at least one of the selected plurality of reads, a fragment end motif, a start position of a fragment for the selected plurality of reads, and an end position of a fragment for the selected plurality of reads.

In some embodiments, a read of the selected plurality of reads is a cfDNA fragment. In some embodiments, the statistical model is configured to receive one or more additional features related to the alteration. In some embodiments, the one or more additional features comprise an alteration depth, an allele frequency of the selected plurality of reads, an alteration coding type, a somatic determination, a germline determination, and a zygosity determination, an age of the individual, a blood tumor mutational burden score, a protein level data, a gene level data, an odds ratio describing an enrichment of the alteration in older patients over younger patients adjusted by disease group, a p-value describing a significance of an enrichment of the alteration in older patients over younger patients adjusted by disease group, an odds ratio describing an enrichment for a specific amino acid change in older patients over younger patients adjusted by disease group, a p-value describing a significance of an enrichment for a specific amino acid change in older patients over younger patients adjusted by disease group, an odds ratio describing an enrichment of the alteration in a certain cancer type compared to other cancers, a p-value describing a significance of an enrichment of the alteration in a certain cancer type compared to other cancers, an odds ratio describing an enrichment for a specific amino acid change in a certain cancer type compared to other cancers, a p-value describing a significance of an enrichment for a specific amino acid change in a certain cancer type compared to other cancers, a tumor fraction, an inferred subclonal status, a distribution of variant level germline, somatic, subclonal somatic calls, variant allele frequency changes over time, a sample level mutational signature score, a description of pathogenicity measures, or any combination thereof.

In some embodiments, the one or more predetermined thresholds comprise a first predetermined threshold, and wherein predicting the origin of the alteration in the sample by comparing the score and one or more predefined thresholds further comprises: comparing, using the one or more processors, the score against the first predetermined threshold and in accordance with a determination that the score is greater than or equal to the first predetermined threshold, determining, using the one or more processors, that the alteration is not derived from a tumor. In such embodiments, the one or more predetermined thresholds comprise a second predetermined threshold, and wherein predicting, using the one or more processors, the origin of the alteration in the sample by comparing the score and one or more predefined thresholds further comprises: comparing, using the one or more processors, the score against the second predetermined threshold; and in accordance with a determination that the score is less than or equal to the second predetermined threshold, determining, using the one or more processors, that the alteration is derived from the tumor. In some embodiments, the second predetermined threshold is the same as the first predetermined threshold.

In one or more embodiments, the individual is suspected of having or is determined to have cancer. In one or more embodiments, the cancer is a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

In one or more embodiments, the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR/MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia. In such embodiments, the method further comprises treating the subject with an anti-cancer therapy.

In one or more embodiments, the anti-cancer therapy comprises a targeted anti-cancer therapy. In such embodiments, the targeted anti-cancer therapy comprises abemaciclib (Verzenio), abiraterone acetate (Zytiga), acalabrutinib (Calquence), ado-trastuzumab emtansine (Kadcyla), afatinib dimaleate (Gilotrif), aldesleukin (Proleukin), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), copanlisib hydrochloride (Aliqopa), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), iobenguane I131 (Azedra), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), panobinostat (Farydak), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sipuleucel-T (Provenge), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.

In one or more embodiments, the method further comprises obtaining the sample from the subject. In one or more embodiments, the sample comprises a tissue biopsy sample, a liquid biopsy sample, or a normal control. In one or more embodiments, the sample is a liquid biopsy sample and comprises blood, plasma, cerebrospinal fluid, sputum, stool, urine, or saliva. In one or more embodiments, the sample is a liquid biopsy sample and comprises circulating tumor cells (CTCs). In one or more embodiments, the sample is a liquid biopsy sample and comprises cell-free DNA (cfDNA), circulating tumor DNA (ctDNA), or any combination thereof.

In one or more embodiments, the plurality of nucleic acid molecules comprises a mixture of tumor nucleic acid molecules and non-tumor nucleic acid molecules. In such embodiments, the tumor nucleic acid molecules are derived from a tumor portion of a heterogeneous tissue biopsy sample, and the non-tumor nucleic acid molecules are derived from a normal portion of the heterogeneous tissue biopsy sample. In one or more embodiments, the sample comprises a liquid biopsy sample, and wherein the tumor nucleic acid molecules are derived from a circulating tumor DNA (ctDNA) fraction of the liquid biopsy sample, and the non-tumor nucleic acid molecules are derived from a non-tumor, cell-free DNA (cfDNA) fraction of the liquid biopsy sample.

In one or more embodiments, the one or more adapters comprise amplification primers, flow cell adaptor sequences, substrate adapter sequences, or sample index sequences. In one or more embodiments, the captured nucleic acid molecules are captured from the amplified nucleic acid molecules by hybridization to one or more bait molecules. In such embodiments, the one or more bait molecules comprise one or more nucleic acid molecules, each comprising a region that is complementary to a region of a captured nucleic acid molecule.

In one or more embodiments, amplifying nucleic acid molecules comprises performing a polymerase chain reaction (PCR) amplification technique, a non-PCR amplification technique, or an isothermal amplification technique. In one or more embodiments, the sequencing comprises use of a massively parallel sequencing (MPS) technique, whole genome sequencing (WGS), whole exome sequencing, targeted sequencing, direct sequencing, or Sanger sequencing technique. In such embodiments, the sequencing comprises massively parallel sequencing, and the massively parallel sequencing technique comprises next generation sequencing (NGS). In one or more embodiments, the sequencer comprises a next generation sequencer.

In one or more embodiments, one or more of the plurality of sequencing reads overlap one or more gene loci within one or more subgenomic intervals in the sample. In such embodiments, the one or more gene loci comprises between 10 and 20 loci, between 10 and 40 loci, between 10 and 60 loci, between 10 and 80 loci, between 10 and 100 loci, between 10 and 150 loci, between 10 and 200 loci, between 10 and 250 loci, between 10 and 300 loci, between 10 and 350 loci, between 10 and 400 loci, between 10 and 450 loci, between 10 and 500 loci, between 20 and 40 loci, between 20 and 60 loci, between 20 and 80 loci, between 20 and 100 loci, between 20 and 150 loci, between 20 and 200 loci, between 20 and 250 loci, between 20 and 300 loci, between 20 and 350 loci, between 20 and 400 loci, between 20 and 500 loci, between 40 and 60 loci, between 40 and 80 loci, between 40 and 100 loci, between 40 and 150 loci, between 40 and 200 loci, between 40 and 250 loci, between 40 and 300 loci, between 40 and 350 loci, between 40 and 400 loci, between 40 and 500 loci, between 60 and 80 loci, between 60 and 100 loci, between 60 and 150 loci, between 60 and 200 loci, between 60 and 250 loci, between 60 and 300 loci, between 60 and 350 loci, between 60 and 400 loci, between 60 and 500 loci, between 80 and 100 loci, between 80 and 150 loci, between 80 and 200 loci, between 80 and 250 loci, between 80 and 300 loci, between 80 and 350 loci, between 80 and 400 loci, between 80 and 500 loci, between 100 and 150 loci, between 100 and 200 loci, between 100 and 250 loci, between 100 and 300 loci, between 100 and 350 loci, between 100 and 400 loci, between 100 and 500 loci, between 150 and 200 loci, between 150 and 250 loci, between 150 and 300 loci, between 150 and 350 loci, between 150 and 400 loci, between 150 and 500 loci, between 200 and 250 loci, between 200 and 300 loci, between 200 and 350 loci, between 200 and 400 loci, between 200 and 500 loci, between 250 and 300 loci, between 250 and 350 loci, between 250 and 400 loci, between 250 and 500 loci, between 300 and 350 loci, between 300 and 400 loci, between 300 and 500 loci, between 350 and 400 loci, between 350 and 500 loci, or between 400 and 500 loci.

In one or more embodiments, the one or more gene loci comprise ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOTIL, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MSTIR, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCDILG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSCIL1, WT1, XPO1, XRCC2, ZNF217, ZNF703, or any combination thereof.

In one or more embodiments, the one or more gene loci comprise ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1β, IL-6, IL-6R, JAK1, JAK2, JAK3, KIT, KRAS, MEK, MET, MSI-H, mTOR, PARP, PD-1, PDGFR, PDGFRα, PDGFRB, PD-L1, PI3Kδ, PIGF, PTCH, RAF, RANKL, RET, ROS1, SLAMF7, VEGF, VEGFA, VEGFB, or any combination thereof.

In one or more embodiments, the method further comprises generating, by the one or more processors, a report indicating the origin of the alteration in the sample. In such embodiments the method further comprises transmitting the report to a healthcare provider. In one or more embodiments, the report is transmitted via a computer network or a peer-to-peer connection.

Embodiments of the present disclosure further provide a method for predicting an origin of an alteration in a sample from an individual, the method comprising: receiving, using one or more processors, sequence read data associated with the sample, selecting, using the one or more processors, a plurality of reads from the sequence read data based on the alteration, determining, using the one or more processors, at least one feature characterizing the selected plurality of reads, inputting, using the one or more processors, the at least one feature into a statistical model, generating, using the one or more processors, a score indicative of the origin of the alteration by the statistical model, and predicting, using the one or more processors, the origin of the alteration in the sample by comparing the score and one or more predefined thresholds.

In one or more embodiments, the alteration includes at least one of an insertion, a deletion, or a substitution. In one or more embodiments, the statistical model is a trained statistical model or an untrained statistical model. In one or more embodiments, the statistical model is a machine learning model. In one or more embodiments, the statistical model is a trained machine learning model and trained by: receiving, using the one or more processors, training data including information quantifying features related to the alteration, and training, using the one or more processors, the machine learning model based on the training data.

In one or more embodiments, the score is indicative of a probability that the alteration is derived from a solid tumor. In one or more embodiments, the score is further indicative of a probability that the alteration is derived from clonal hematopoiesis.

In one or more embodiments, the at least one feature comprises a fragment length for the selected plurality of reads, a start position of a fragment for the selected plurality of reads, an end position of a fragment for the selected plurality of reads, or a combination thereof. In one or more embodiments, the at least one feature comprises at least one fragmentomic characteristic of the sample. In such embodiments, the at least one fragmentomic characteristic of the sample comprises at least one of: an amount of the selected plurality of reads having a specified length, a mean fragment length of the selected plurality of reads, a median fragment length of the selected plurality of reads, an interquartile range of fragment lengths of the selected plurality of reads, a distribution of fragment lengths of the selected plurality of reads, one or more peaks of fragment lengths for the selected plurality of reads, a fragment length for at least one of the selected plurality of reads, a fragment end motif, a start position of a fragment for the selected plurality of reads, and an end position of a fragment for the selected plurality of reads. In such embodiments, a read of the selected plurality of reads is a cfDNA fragment.

In one or more embodiments, the statistical model is configured to receive one or more additional features related to the alteration. In such embodiments, the one or more additional features comprise an alteration depth, an allele frequency of the selected plurality of reads, an alteration coding type, a somatic determination, a germline determination, a zygosity determination, an age of the individual, a blood tumor mutational burden score, a protein level data, a gene level data, an odds ratio describing an enrichment of the alteration in older patients over younger patients adjusted by disease group, a p-value describing a significance of an enrichment of the alteration in older patients over younger patients adjusted by disease group, an odds ratio describing an enrichment for a specific amino acid change in older patients over younger patients adjusted by disease group, a p-value describing a significance of an enrichment for a specific amino acid change in older patients over younger patients adjusted by disease group, an odds ratio describing an enrichment of the alteration in a certain cancer type compared to other cancers, a p-value describing a significance of an enrichment of the alteration in a certain cancer type compared to other cancers, an odds ratio describing an enrichment for a specific amino acid change in a certain cancer type compared to other cancers, a p-value describing a significance of an enrichment for a specific amino acid change in a certain cancer type compared to other cancers, a tumor fraction, an inferred subclonal status, a distribution of variant level germline, somatic, subclonal somatic calls, variant allele frequency changes over time, a sample level mutational signature score, a description of pathogenicity measures, or any combination thereof. In such embodiments, one or more of the somatic determination, the germline determination, and the zygosity determination is a computational determination.

In one or more embodiments, the one or more predetermined thresholds comprise a first predetermined threshold, and wherein predicting the origin of the alteration in the sample by comparing the score and one or more predefined thresholds further comprises: comparing, using the one or more processors, the score against the first predetermined threshold, and in accordance with a determination that the score is greater than or equal to the first predetermined threshold, determining, using the one or more processors, that the alteration is not derived from a tumor.

In one or more embodiments, the one or more predetermined thresholds comprise a second predetermined threshold, and wherein predicting, using the one or more processors, the origin of the alteration in the sample by comparing the score and one or more predefined thresholds further comprises: comparing, using the one or more processors, the score against the second predetermined threshold, and in accordance with a determination that the score is less than or equal to the second predetermined threshold, determining, using the one or more processors, that the alteration is derived from the tumor. In such embodiments, the second predetermined threshold is the same as the first predetermined threshold. In one or more embodiments, the second predetermined threshold is different from the first predetermined threshold.

In one or more embodiments, predicting the origin of the alteration in the sample by comparing the score and one or more predefined thresholds further comprises: in accordance with a determination that the score is less than the first predetermined threshold and that the score is greater than the second predetermined threshold, determining, using the one or more processors, that the score is inconclusive.

In one or more embodiments, the one or more predetermined thresholds are determined by maximizing or minimizing one or more of a function of sensitivity and specificity and the area under a predictor's receiver operating characteristic curve. In one or more embodiments, the sample comprises a liquid biopsy sample and comprises cell-free DNA (cfDNA), circulating tumor DNA (ctDNA), or any combination thereof.

In one or more embodiments, the method further comprises identifying, using the one or more processors, one or more of a treatment or a monitoring requirement for the individual based on the prediction. In such embodiments, the monitoring requirement comprises: obtaining, using the one or more processors, an additional sample from the individual and determining, using the one or more processors, an origin of the alteration in the additional sample using one or more tests. In such embodiments, the one or more tests comprises at least one of a paired normal test or an orthogonal test. In one or more embodiments, the paired normal test comprises sequencing peripheral blood mononuclear cells. In one or more embodiments, the one or more tests comprises reflex testing of a tissue sample.

In one or more embodiments, the method further comprises administering, using the one or more processors, one or more of a treatment or a monitoring type for the individual based on the prediction. In such embodiments, administering the monitoring type comprises: obtaining, using the one or more processors, an additional sample from the individual, and determining, using the one or more processors, an origin of the alteration in the additional sample using one or more tests.

In one or more embodiments, the method further comprises determining, using the one or more processors, an adequacy of the sample for clinical decision-making. In such embodiments, the sample is determined to be inadequate for clinical decision-making if the alteration is not derived from a tumor.

In one or more embodiments, the method further comprises determining one or more biomarkers based on the score. In such embodiments, the one or more biomarkers comprise at least one of: a tumor fraction or a blood tumor mutational burden (bTMB).

In one or more embodiments, the method further comprises obtaining training data, wherein the training data includes information quantifying features related to the alteration. In such embodiments, the information quantifying features related to the alteration includes at least one of: fragmentomic characteristics for each of a plurality of training sequencing reads, additional features for the plurality of training sequencing reads, and origin information for the plurality of training sequence reads. In one or more embodiments, the fragmentomic characteristics for each of a plurality of training sequencing reads comprises at least one of: an amount of a fragment having a specified length, a mean fragment length of the selected plurality of reads, a median fragment length of the selected plurality of reads, an interquartile range of the selected plurality of reads, a distribution of a fragment length of the selected plurality of reads, one or more peaks of a fragment length for the selected plurality of reads, a fragment length for the selected plurality of reads, a start position of a fragment for the selected plurality of reads, and an end position of a fragment for the selected plurality of reads. In one or more embodiments, the additional features for each of the plurality of training sequence reads comprise an alteration depth, an allele frequency of the selected plurality of reads, an alteration coding type, a somatic determination, a germline determination, a zygosity determination, an age of the individual, a blood tumor mutational burden score, a protein level data, a gene level data, or any combination thereof.

In one or more embodiments, the training data is obtained by obtaining, using the one or more processors, a matched sample pair comprising a white blood cell sample and a corresponding plasma sample, receiving, using the one or more processors, white blood cell read data obtained from the white blood cell sample and plasma read data obtained from the plasma sample, selecting, using the one or more processors, a plurality of training sequence reads from the white blood cell read data based on the alteration and further selecting a plurality of training sequence reads from the plasma read data based on the alteration, determining, using the one or more processors, the information quantifying features related to the alteration from the plasma read data and white blood cell read data, and determining, using the one or more processors, origin information based on a comparison of the selected plurality of training sequence reads from the plasma read data and the selected plurality of training sequence reads from white blood cell read data.

In one or more embodiments, the training data is obtained by: receiving, using one or more processors, sequence read data obtained based on a training sample from a subject, selecting, using the one or more processors, a plurality of training sequence reads from the sequence read data obtained based on the training sample based on a gene associated with the alteration, classifying, using one or more processors, the plurality of training sequence reads into a first category corresponding to the alteration and a second category corresponding to a wild-type, determining, using one or more processors, a plurality of first fragment length metrics for the first category, determining, using one or more processors, a plurality of second fragment length metrics for the second category, wherein the information quantifying features related to the alteration comprises the plurality of first fragment length metrics and the plurality of second fragment length metrics.

In such embodiments, the training further comprises: inputting, using one or more processors, the information quantifying features related to the alteration into the statistical model, obtaining, using one or more processors, a feature vector based on the first plurality of fragment length metrics and the second plurality of fragment length metrics, determining, using one or more processors, a score based on the feature vector and one or more additional features, and updating one or more weights associated with the statistical model based on the score.

In one or more embodiments, a first fragment length metric of the plurality of first fragment length metrics comprises a plurality of first fragment amounts in the first category, each first fragment amount corresponding to a specified length for a plurality of lengths. In one or more embodiments, a second fragment length metric of the plurality of second fragment length metrics comprises a plurality of second fragment amounts in the second category, each second fragment amount having a specified length for the plurality of lengths. In one or more embodiments, the first fragment length metrics correspond to the alteration and the second fragment length metrics correspond to the wild-type. In one or more embodiments, the plurality of first fragment length metrics and the plurality of second fragment length metrics are stored in a two-dimensional array. In one or more embodiments, the feature vector includes one or more fragmentomic characteristics of the plurality of training sequence reads, one or more additional features of the plurality of training sequence reads, or a combination thereof.

In one or more embodiments, the statistical model is part of a machine learning process. In one or more embodiments, the statistical model includes an artificial intelligence learning model. In one or more embodiments, the statistical model is at least one of a Bayesian model, a random forest model, a support vector machine learning model, a linear regression model, a non-linear regression model, a multivariate regression machine learning model, a robust machine learning model, a neural network model, a nearest neighbor machine learning model, a gradient boosting ensemble model, and a proportional hazards model.

In one or more embodiments, the statistical model is a convolutional neural network (CNN) machine learning model. In one or more embodiments, the CNN machine learning model comprises one or more one or more convolution layers, one or more pooling layers, one or more dropout layers, one or more dense layers, or a combination thereof. In one or more embodiments, the CNN machine learning model is configured to receive, an input, at least one fragmentomic characteristic of the sample and at least one additional feature. In such embodiments, the method further comprises processing the at least one fragmentomic characteristic of the sample with the CNN machine learning model, concatenating an output of the CNN machine learning model with the one additional feature, and inputting the concatenated output into a deep neural network, wherein generating the score indicative of the origin of the alteration is performed by the deep neural network.

In one or more embodiments, the CNN machine learning model is configured to extract one or more additional fragmentomic characteristics from the input. In one or more embodiments, the at least one additional feature comprises an age of the individual, a blood tumor mutational burden score, a protein level data, a gene level data, or any combination thereof.

In one or more embodiments, the statistical model is a supervised machine learning model or an unsupervised machine learning model.

In one or more embodiments, the method further comprises selecting, using the one or more processors, a plurality of reference reads from the sequence read data based on a location of a reference gene associated with the alteration, determining, using the one or more processors, at least one feature characterizing the selected plurality of reference reads, comparing, using the one or more processors, the at least one feature characterizing the selected plurality of reference reads and the at least one feature characterizing the selected plurality of reads to determine a reference score, and inputting, using the one or more processors, the reference score into the statistical model.

In one or more embodiments, the alteration is based on a predetermined user input. In one or more embodiments, the alteration is determined based on an algorithmic process. In one or more embodiments, the statistical model is configured to determine one or more fragmentomic characteristics based on the at least one feature characterizing the selected plurality of reads.

In one or more embodiments, the sequence read data is obtained through the use of next-generation sequencing. In one or more embodiments, selecting a plurality of reads from the sequence read data based on the alteration comprises selecting sequencing reads from a genomic region where the alteration is located.

Embodiments of the present disclosure provide methods for predicting an origin of an alteration in a sample from an individual, the method comprising: receiving, using one or more processors, sequence read data associated with the sample, selecting, using the one or more processors, a plurality of reads from the sequence read data based on the alteration, determining, using the one or more processors, at least one at least one fragmentomic characteristic based on the selected plurality of reads, inputting, using the one or more processors, the at least one fragmentomic characteristic into a statistical model, generating, using the one or more processors, a score indicative of the origin of the alteration by the statistical model, and predicting, using the one or more processors, the origin of the alteration in the sample by comparing the score and one or more predefined thresholds.

In one or more embodiments, the alteration includes at least one of an insertion, a deletion, or a substitution. In one or more embodiments, the statistical model is a trained statistical model or an untrained statistical model. In one or more embodiments, statistical model is a machine learning model.

In one or more embodiments, the statistical model is a trained machine learning model and trained by: receiving, using the one or more processors, training data including information quantifying features related to the alteration and training, using the one or more processors, the machine learning model based on the training data.

In one or more embodiments, the score is indicative of a probability that the alteration is derived from a solid tumor. In one or more embodiments, the score is further indicative of a probability that the alteration is derived from clonal hematopoiesis.

In one or more embodiments, the at least one feature comprises at least one fragmentomic characteristic of the sample. In such embodiments, the at least one fragmentomic characteristic of the sample comprises at least one of: an amount of the selected plurality of reads having a specified length, a mean fragment length of the selected plurality of reads, a median fragment length of the selected plurality of reads, an interquartile range of fragment lengths of the selected plurality of reads, a distribution of fragment lengths of the selected plurality of reads, one or more peaks of fragment lengths for the selected plurality of reads, a fragment length for at least one of the selected plurality of reads, a fragment end motif, a start position of a fragment for the selected plurality of reads, and an end position of a fragment for the selected plurality of reads. In such embodiments, a read of the selected plurality of reads is a cfDNA fragment.

In one or more embodiments, the statistical model is configured to receive one or more additional features related to the alteration. In such embodiments, the one or more additional features comprise an alteration depth, an allele frequency of the selected plurality of reads, an alteration coding type, a somatic determination, a germline determination, a zygosity determination, an age of the individual, a blood tumor mutational burden score, a protein level data, a gene level data, an odds ratio describing an enrichment of the alteration in older patients over younger patients adjusted by disease group, a p-value describing a significance of an enrichment of the alteration in older patients over younger patients adjusted by disease group, an odds ratio describing an enrichment for a specific amino acid change in older patients over younger patients adjusted by disease group, a p-value describing a significance of an enrichment for a specific amino acid change in older patients over younger patients adjusted by disease group, an odds ratio describing an enrichment of the alteration in a certain cancer type compared to other cancers, a p-value describing a significance of an enrichment of the alteration in a certain cancer type compared to other cancers, an odds ratio describing an enrichment for a specific amino acid change in a certain cancer type compared to other cancers, a p-value describing a significance of an enrichment for a specific amino acid change in a certain cancer type compared to other cancers, a tumor fraction, an inferred subclonal status, a distribution of variant level germline, somatic, subclonal somatic calls, variant allele frequency changes over time, a sample level mutational signature score, a description of pathogenicity measures, or any combination thereof. In such embodiments, one or more of the somatic determination, the germline determination, and the zygosity determination is a computational determination.

In one or more embodiments, the one or more predetermined thresholds comprise a first predetermined threshold, and wherein predicting the origin of the alteration in the sample by comparing the score and one or more predefined thresholds further comprises: comparing, using the one or more processors, the score against the first predetermined threshold; and in accordance with a determination that the score is greater than or equal to the first predetermined threshold, determining, using the one or more processors, that the alteration is not derived from a tumor.

In one or more embodiments, the one or more predetermined thresholds comprise a second predetermined threshold, and wherein predicting, using the one or more processors, the origin of the alteration in the sample by comparing the score and one or more predefined thresholds further comprises: comparing, using the one or more processors, the score against the second predetermined threshold, and in accordance with a determination that the score is less than or equal to the second predetermined threshold, determining, using the one or more processors, that the alteration is derived from the tumor. In such embodiments, the second predetermined threshold is the same as the first predetermined threshold. Further, in such embodiments, the second predetermined threshold is different from the first predetermined threshold.

In one or more embodiments, predicting the origin of the alteration in the sample by comparing the score and one or more predefined thresholds further comprises: in accordance with a determination that the score is less than the first predetermined threshold and that the score is greater than the second predetermined threshold, determining, using the one or more processors, that the score is inconclusive.

In one or more embodiments, the one or more predetermined thresholds are determined by maximizing or minimizing one or more of a function of sensitivity and specificity and the area under a predictor's receiver operating characteristic (AUC). In one or more embodiments, the sample comprises a liquid biopsy sample and comprises cell-free DNA (cfDNA), circulating tumor DNA (ctDNA), or any combination thereof.

In one or more embodiments, the method further comprises identifying, using the one or more processors, one or more of a treatment or a monitoring requirement for the individual based on the prediction. In one or more embodiments, the monitoring requirement further comprises: obtaining, using the one or more processors, an additional sample from the individual; and determining, using the one or more processors, an origin of the alteration in the additional sample using one or more tests. In such embodiments, the one or more tests comprises at least one of a paired normal test or an orthogonal test. In one or more embodiments, the paired normal test comprises sequencing peripheral blood mononuclear cells. In one or more embodiments, the one or more tests comprises reflex testing of a tissue sample.

In one or more embodiments, the method further comprises administering, using the one or more processors, one or more of a treatment or a monitoring type for the individual based on the prediction. In such embodiments, administering the monitoring type comprises: obtaining, using the one or more processors, an additional sample from the individual, and determining, using the one or more processors, an origin of the alteration in the additional sample using one or more tests.

In one or more embodiments, the method further comprises determining, using the one or more processors, an adequacy of the sample for clinical decision-making. In such embodiments, the sample is determined to be inadequate for clinical decision-making if the alteration is not derived from a tumor.

In one or more embodiments, the method further comprises determining one or more biomarkers based on the score. In such embodiments, the one or more biomarkers comprise at least one of: a tumor fraction or a blood tumor mutational burden (bTMB).

In one or more embodiments, the method further comprises obtaining training data, wherein the training data includes information quantifying features related to the alteration. In such embodiments, the information quantifying features related to the alteration includes at least one of: fragmentomic characteristics for each of a plurality of training sequencing reads, additional features for the plurality of training sequencing reads, and origin information for the plurality of training sequence reads. In one or more embodiments, the fragmentomic characteristics for each of a plurality of training sequencing reads comprises at least one of: an amount of a fragment having a specified length, a mean fragment length of the selected plurality of reads, a median fragment length of the selected plurality of reads, an interquartile range of the selected plurality of reads, a distribution of a fragment length of the selected plurality of reads, one or more peaks of a fragment length for the selected plurality of reads, a fragment length for the selected plurality of reads, a start position of a fragment for the selected plurality of reads, and an end position of a fragment for the selected plurality of reads. In one or more embodiments, the additional features for each of the plurality of training sequence reads comprise an alteration depth, an allele frequency of the selected plurality of reads, an alteration coding type, a somatic determination, a germline determination, a zygosity determination, an age of the individual, a blood tumor mutational burden score, a protein level data, a gene level data, or any combination thereof.

In one or more embodiments, the training data is obtained by obtaining, using the one or more processors, a matched sample pair comprising a white blood cell sample and a corresponding plasma sample, receiving, using the one or more processors, white blood cell read data obtained from the white blood cell sample and plasma read data obtained from the plasma sample, selecting, using the one or more processors, a plurality of training sequence reads from the white blood cell read data based on the alteration and further selecting a plurality of training sequence reads from the plasma read data based on the alteration, determining, using the one or more processors, the information quantifying features related to the alteration from the plasma read data and white blood cell read data, and determining, using the one or more processors, origin information based on a comparison of the selected plurality of training sequence reads from the plasma read data and the selected plurality of training sequence reads from white blood cell read data.

In one or more embodiments, the training data is obtained by: receiving, using one or more processors, sequence read data obtained based on a training sample from a subject, selecting, using the one or more processors, a plurality of training sequence reads from the sequence read data obtained based on the training sample based on a gene associated with the alteration, classifying, using one or more processors, the plurality of training sequence reads into a first category corresponding to the alteration and a second category corresponding to a wild-type, determining, using one or more processors, a plurality of first fragment length metrics for the first category, determining, using one or more processors, a plurality of second fragment length metrics for the second category, wherein the information quantifying features related to the alteration comprises the plurality of first fragment length metrics and the plurality of second fragment length metrics.

In such embodiments, the training further comprises: inputting, using one or more processors, the information quantifying features related to the alteration into the statistical model, obtaining, using one or more processors, a feature vector based on the first plurality of fragment length metrics and the second plurality of fragment length metrics, determining, using one or more processors, a score based on the feature vector and one or more additional features, and updating one or more weights associated with the statistical model based on the score.

In one or more embodiments, a first fragment length metric of the plurality of first fragment length metrics comprises a plurality of first fragment amounts in the first category, each first fragment amount corresponding to a specified length for a plurality of lengths. In one or more embodiments, a second fragment length metric of the plurality of second fragment length metrics comprises a plurality of second fragment amounts in the second category, each second fragment amount having a specified length for the plurality of lengths. In one or more embodiments, the first fragment length metrics correspond to the alteration and the second fragment length metrics correspond to the wild-type. In one or more embodiments, the plurality of first fragment length metrics and the plurality of second fragment length metrics are stored in a two-dimensional array. In one or more embodiments, the feature vector includes one or more fragmentomic characteristics of the plurality of training sequence reads, one or more additional features of the plurality of training sequence reads, or a combination thereof.

In one or more embodiments, the statistical model is part of a machine learning process. In one or more embodiments, the statistical model includes an artificial intelligence learning model. In one or more embodiments, the statistical model is at least one of a Bayesian model, a random forest model, a support vector machine learning model, a linear regression model, a non-linear regression model, a multivariate regression machine learning model, a robust machine learning model, a neural network model, a nearest neighbor machine learning model, a gradient boosting ensemble model, and a proportional hazards model.

In one or more embodiments, the statistical model is a convolutional neural network (CNN) machine learning model. In one or more embodiments, the CNN machine learning model comprises one or more one or more convolution layers, one or more pooling layers, one or more dropout layers, one or more dense layers, or a combination thereof. In one or more embodiments, the CNN machine learning model is configured to receive, an input, at least one fragmentomic characteristic of the sample and at least one additional feature. In such embodiments, the method further comprises processing the at least one fragmentomic characteristic of the sample with the CNN machine learning model, concatenating an output of the CNN machine learning model with the one additional feature, and inputting the concatenated output into a deep neural network, wherein generating the score indicative of the origin of the alteration is performed by the deep neural network.

In one or more embodiments, the CNN machine learning model is configured to extract one or more additional fragmentomic characteristics from the input. In one or more embodiments, the at least one additional feature comprises an age of the individual, a blood tumor mutational burden score, a protein level data, a gene level data, or any combination thereof.

In one or more embodiments, the statistical model is a supervised machine learning model or an unsupervised machine learning model.

In one or more embodiments, the method further comprises selecting, using the one or more processors, a plurality of reference reads from the sequence read data based on a location of a reference gene associated with the alteration, determining, using the one or more processors, at least one fragmentomic characteristic based on the selected plurality of reference reads, comparing, using the one or more processors, the at least one fragmentomic characteristic based on the selected plurality of reference reads and the at least one fragmentomic characteristic based on the selected plurality of reads to determine a reference score, and inputting, using the one or more processors, the reference score into the statistical model.

In one or more embodiments, the alteration is based on a predetermined user input. In one or more embodiments, the alteration is determined based on an algorithmic process. In one or more embodiments, the statistical model is configured to determine the at least one fragmentomic characteristics based on the selected plurality of reads.

In one or more embodiments, the sequence read data is obtained through the use of next-generation sequencing. In one or more embodiments, selecting a plurality of reads from the sequence read data based on the alteration comprises selecting sequencing reads from a genomic region where the alteration is located.

Embodiments of the present disclosure provide methods for diagnosing a disease, the method comprising: diagnosing that a subject has the disease based on a determination of an origin of an alteration in a sample from the subject, wherein the origin of the alteration is determined according to the method of any of the embodiments of this disclosure.

Embodiments of the present disclosure provide methods of selecting an anti-cancer therapy, the method comprising: responsive to determining an origin of an alteration in a sample from a subject, selecting an anti-cancer therapy for the subject, wherein the origin of the alteration is determined according to the method of any of the embodiments of this disclosure.

Embodiments of the present disclosure provide methods of treating a cancer in a subject, comprising: responsive to determining an origin of an alteration in a sample from the subject, administering an effective amount of an anti-cancer therapy to the subject, wherein the origin of the alteration is determined according to the method of any of the embodiments of this disclosure.

Embodiments of the present disclosure provide methods for monitoring cancer progression or recurrence in a subject, the method comprising determining a first origin of an alteration in a first sample obtained from the subject at a first time point according to the method of any of the embodiments of this disclosure, determining a second origin of an alteration in a second sample obtained from the subject at a second time point, and comparing the first origin of the alteration to the second origin of the alteration, thereby monitoring the cancer progression or recurrence.

In one or more embodiments, the second origin of the alteration for the second sample is determined according to the method of any one of the embodiments described in this disclosure.

In one or more embodiments, the method further comprises selecting an anti-cancer therapy for the subject in response to the cancer progression. In one or more embodiments, the method further comprises administering an anti-cancer therapy to the subject in response to the cancer progression. In one or more embodiments, the method further comprises adjusting an anti-cancer therapy for the subject in response to the cancer progression. In one or more embodiments, the method further comprises adjusting a dosage of the anti-cancer therapy or selecting a different anti-cancer therapy in response to the cancer progression. In one or more embodiments, the method further comprises administering the adjusted anti-cancer therapy to the subject.

In one or more embodiments, the first time point is before the subject has been administered an anti-cancer therapy, and wherein the second time point is after the subject has been administered the anti-cancer therapy. In one or more embodiments, the subject has a cancer, is at risk of having a cancer, is being routine tested for cancer, or is suspected of having a cancer. In one or more embodiments, the cancer is a solid tumor. In one or more embodiments, the cancer is a hematological cancer. In one or more embodiments, the anti-cancer therapy comprises chemotherapy, radiation therapy, immunotherapy, a targeted therapy, or surgery.

Embodiments of the present disclosure provide methods, wherein the determination of the origin of the alteration in the sample is used in making suggested treatment decisions for the subject. Embodiments of the present disclosure provide methods, wherein the determination of the origin of the alteration in the sample is used in applying or administering a treatment to the subject.

Embodiments of the present disclosure are directed to systems comprising: one or more processors and a memory communicatively coupled to the one or more processors. In one or more embodiments, the memory is configured to store instructions that, when executed by the one or more processors, cause the system to: receive, using one or more processors, sequence read data associated with the sample, select, using the one or more processors, a plurality of reads from the sequence read data based on the alteration, determine, using the one or more processors, at least one feature characterizing the selected plurality of reads, input, using the one or more processors, the at least one feature into a statistical model, generate, using the one or more processors, a score indicative of the origin of the alteration by the statistical model, and predict, using the one or more processors, the origin of the alteration in the sample by comparing the score and one or more predefined thresholds.

In some embodiments, the alteration includes at least one of an insertion, a deletion, or a substitution. In some embodiments, the system can further include providing a plurality of nucleic acid molecules obtained from a sample from a subject, ligating one or more adapters onto one or more nucleic acid molecules from the plurality of nucleic acid molecules, amplifying the one or more ligated nucleic acid molecules from the plurality of nucleic acid molecules, capturing amplified nucleic acid molecules from the amplified nucleic acid molecules, sequencing, by a sequencer, the captured nucleic acid molecules to obtain a plurality of sequence reads that represent the captured nucleic acid molecules, receiving, at one or more processors, sequence read data for the plurality of sequence reads, receiving, using one or more processors, sequence read data associated with the sample, selecting, using the one or more processors, a plurality of reads from the sequence read data based on the alteration, determining, using the one or more processors, at least one feature characterizing the selected plurality of reads, inputting, using the one or more processors, the at least one feature into a statistical model, generating, using the one or more processors, a score indicative of the origin of the alteration by the statistical model, and predicting, using the one or more processors, the origin of the alteration in the sample by comparing the score and one or more predefined thresholds.

In some embodiments, the statistical model is a trained statistical model or an untrained statistical model. In some embodiments, the statistical model is a trained machine learning model and trained by: receiving, using the one or more processors, training data including information quantifying features related to the alteration; and training, using the one or more processors, the machine learning model based on the training data.

In some embodiments, the score is indicative of a probability that the alteration is derived from a solid tumor. In some embodiments, the score is further indicative of a probability that the alteration is derived from clonal hematopoiesis.

In some of embodiments, the at least one feature comprises at least one fragmentomic characteristic of the sample. In some embodiments, the at least one fragmentomic characteristic of the sample comprises at least one of: an amount of the selected plurality of reads having a specified length, a mean fragment length of the selected plurality of reads, a median fragment length of the selected plurality of reads, an interquartile range of fragment lengths of the selected plurality of reads, a distribution of fragment lengths of the selected plurality of reads, one or more peaks of fragment lengths for the selected plurality of reads, a fragment length for at least one of the selected plurality of reads, a fragment end motif, a start position of a fragment for the selected plurality of reads, and an end position of a fragment for the selected plurality of reads.

In some embodiments, a read of the selected plurality of reads is a cfDNA fragment. In some embodiments, the statistical model is configured to receive one or more additional features related to the alteration. In some embodiments, the one or more additional features comprise an alteration depth, an allele frequency of the selected plurality of reads, an alteration coding type, a somatic determination, a germline determination, and a zygosity determination, an age of the individual, a blood tumor mutational burden score, a protein level data, a gene level data, an odds ratio describing an enrichment of the alteration in older patients over younger patients adjusted by disease group, a p-value describing a significance of an enrichment of the alteration in older patients over younger patients adjusted by disease group, an odds ratio describing an enrichment for a specific amino acid change in older patients over younger patients adjusted by disease group, a p-value describing a significance of an enrichment for a specific amino acid change in older patients over younger patients adjusted by disease group, an odds ratio describing an enrichment of the alteration in a certain cancer type compared to other cancers, a p-value describing a significance of an enrichment of the alteration in a certain cancer type compared to other cancers, an odds ratio describing an enrichment for a specific amino acid change in a certain cancer type compared to other cancers, a p-value describing a significance of an enrichment for a specific amino acid change in a certain cancer type compared to other cancers, a tumor fraction, an inferred subclonal status, a distribution of variant level germline, somatic, subclonal somatic calls, variant allele frequency changes over time, a sample level mutational signature score, a description of pathogenicity measures, or any combination thereof.

In some embodiments, the one or more predetermined thresholds comprise a first predetermined threshold, and wherein predicting the origin of the alteration in the sample by comparing the score and one or more predefined thresholds further comprises: comparing, using the one or more processors, the score against the first predetermined threshold and in accordance with a determination that the score is greater than or equal to the first predetermined threshold, determining, using the one or more processors, that the alteration is not derived from a tumor. In such embodiments, the one or more predetermined thresholds comprise a second predetermined threshold, and wherein predicting, using the one or more processors, the origin of the alteration in the sample by comparing the score and one or more predefined thresholds further comprises: comparing, using the one or more processors, the score against the second predetermined threshold; and in accordance with a determination that the score is less than or equal to the second predetermined threshold, determining, using the one or more processors, that the alteration is derived from the tumor. In some embodiments, the second predetermined threshold is the same as the first predetermined threshold.

In one or more embodiments, the statistical model is part of a machine learning process. In one or more embodiments, the statistical model includes an artificial intelligence learning model. In one or more embodiments, the statistical model is at least one of a Bayesian model, a random forest model, a support vector machine learning model, a linear regression model, a non-linear regression model, a multivariate regression machine learning model, a robust machine learning model, a neural network model, a nearest neighbor machine learning model, a gradient boosting ensemble model, and a proportional hazards model. In one or more embodiments, the statistical model is a convolutional neural network (CNN) machine learning model. In one or more embodiments, the CNN machine learning model comprises one or more one or more convolution layers, one or more pooling layers, one or more dropout layers, one or more dense layers, or a combination thereof.

Embodiments of the present disclosure are directed to non-transitory computer-readable storage mediums storing one or more programs. In one or more embodiments, the one or more programs comprising instructions, which when executed by one or more processors of a system, cause the system to: receive, using one or more processors, sequence read data associated with the sample, select, using the one or more processors, a plurality of reads from the sequence read data based on the alteration, determine, using the one or more processors, at least one feature characterizing the selected plurality of reads, input, using the one or more processors, the at least one feature into a statistical model, generate, using the one or more processors, a score indicative of the origin of the alteration by the statistical model, and predict, using the one or more processors, the origin of the alteration in the sample by comparing the score and one or more predefined thresholds.

In some embodiments, the alteration includes at least one of an insertion, a deletion, or a substitution. In some embodiments, the non-transitory computer readable storage medium can further include providing a plurality of nucleic acid molecules obtained from a sample from a subject, ligating one or more adapters onto one or more nucleic acid molecules from the plurality of nucleic acid molecules, amplifying the one or more ligated nucleic acid molecules from the plurality of nucleic acid molecules, capturing amplified nucleic acid molecules from the amplified nucleic acid molecules, sequencing, by a sequencer, the captured nucleic acid molecules to obtain a plurality of sequence reads that represent the captured nucleic acid molecules, receiving, at one or more processors, sequence read data for the plurality of sequence reads, receiving, using one or more processors, sequence read data associated with the sample, selecting, using the one or more processors, a plurality of reads from the sequence read data based on the alteration, determining, using the one or more processors, at least one feature characterizing the selected plurality of reads, inputting, using the one or more processors, the at least one feature into a statistical model, generating, using the one or more processors, a score indicative of the origin of the alteration by the statistical model, and predicting, using the one or more processors, the origin of the alteration in the sample by comparing the score and one or more predefined thresholds.

In some embodiments, the statistical model is a trained statistical model or an untrained statistical model. In some embodiments, the statistical model is a trained machine learning model and trained by: receiving, using the one or more processors, training data including information quantifying features related to the alteration; and training, using the one or more processors, the machine learning model based on the training data.

In some embodiments, the score is indicative of a probability that the alteration is derived from a solid tumor. In some embodiments, the score is further indicative of a probability that the alteration is derived from clonal hematopoiesis.

In some of embodiments, the at least one feature comprises at least one fragmentomic characteristic of the sample. In some embodiments, the at least one fragmentomic characteristic of the sample comprises at least one of: an amount of the selected plurality of reads having a specified length, a mean fragment length of the selected plurality of reads, a median fragment length of the selected plurality of reads, an interquartile range of fragment lengths of the selected plurality of reads, a distribution of fragment lengths of the selected plurality of reads, one or more peaks of fragment lengths for the selected plurality of reads, a fragment length for at least one of the selected plurality of reads, a fragment end motif, a start position of a fragment for the selected plurality of reads, and an end position of a fragment for the selected plurality of reads.

In some embodiments, a read of the selected plurality of reads is a cfDNA fragment. In some embodiments, the statistical model is configured to receive one or more additional features related to the alteration. In some embodiments, the one or more additional features comprise an alteration depth, an allele frequency of the selected plurality of reads, an alteration coding type, a somatic determination, a germline determination, and a zygosity determination, an age of the individual, a blood tumor mutational burden score, a protein level data, a gene level data, an odds ratio describing an enrichment of the alteration in older patients over younger patients adjusted by disease group, a p-value describing a significance of an enrichment of the alteration in older patients over younger patients adjusted by disease group, an odds ratio describing an enrichment for a specific amino acid change in older patients over younger patients adjusted by disease group, a p-value describing a significance of an enrichment for a specific amino acid change in older patients over younger patients adjusted by disease group, an odds ratio describing an enrichment of the alteration in a certain cancer type compared to other cancers, a p-value describing a significance of an enrichment of the alteration in a certain cancer type compared to other cancers, an odds ratio describing an enrichment for a specific amino acid change in a certain cancer type compared to other cancers, a p-value describing a significance of an enrichment for a specific amino acid change in a certain cancer type compared to other cancers, a tumor fraction, an inferred subclonal status, a distribution of variant level germline, somatic, subclonal somatic calls, variant allele frequency changes over time, a sample level mutational signature score, a description of pathogenicity measures, or any combination thereof.

In some embodiments, the one or more predetermined thresholds comprise a first predetermined threshold, and wherein predicting the origin of the alteration in the sample by comparing the score and one or more predefined thresholds further comprises: comparing, using the one or more processors, the score against the first predetermined threshold and in accordance with a determination that the score is greater than or equal to the first predetermined threshold, determining, using the one or more processors, that the alteration is not derived from a tumor. In such embodiments, the one or more predetermined thresholds comprise a second predetermined threshold, and wherein predicting, using the one or more processors, the origin of the alteration in the sample by comparing the score and one or more predefined thresholds further comprises: comparing, using the one or more processors, the score against the second predetermined threshold; and in accordance with a determination that the score is less than or equal to the second predetermined threshold, determining, using the one or more processors, that the alteration is derived from the tumor. In some embodiments, the second predetermined threshold is the same as the first predetermined threshold.

In one or more embodiments, the statistical model is part of a machine learning process. In one or more embodiments, the statistical model includes an artificial intelligence learning model. In one or more embodiments, the statistical model is at least one of a Bayesian model, a random forest model, a support vector machine learning model, a linear regression model, a non-linear regression model, a multivariate regression machine learning model, a robust machine learning model, a neural network model, a nearest neighbor machine learning model, a gradient boosting ensemble model, and a proportional hazards model. In one or more embodiments, the statistical model is a convolutional neural network (CNN) machine learning model. In one or more embodiments, the CNN machine learning model comprises one or more one or more convolution layers, one or more pooling layers, one or more dropout layers, one or more dense layers, or a combination thereof.

Embodiments of the present disclosure are directed to methods for predicting an origin of an alteration in a sample from an individual, the method comprising: receiving, using one or more processors, sequence read data associated with the sample, selecting, using the one or more processors, a plurality of reads from the sequence read data based on the alteration, determining, using the one or more processors, at least one feature characterizing the selected plurality of reads, inputting, using the one or more processors, the at least one feature into a trained statistical model, generating, using the one or more processors, a score indicative of the origin of the alteration by the trained statistical model, and predicting, using the one or more processors, the origin of the alteration in the sample by comparing the score and one or more predefined thresholds.

Embodiments of the present disclosure are directed to methods for predicting an origin of an alteration in a sample from an individual, the method comprising receiving, using one or more processors, sequence read data associated with the sample, selecting, using the one or more processors, a plurality of reads from the sequence read data based on the alteration, determining, using the one or more processors, at least one feature characterizing the selected plurality of reads, inputting, using the one or more processors, the at least one feature into a trained statistical model, generating, using the one or more processors, a score indicative of the origin of the alteration by the trained statistical model, wherein the score is indicative of a probability that the alteration is derived from a tumor, and predicting, using the one or more processors, the origin of the alteration in the sample by comparing the score and one or more predefined thresholds.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### BRIEF DESCRIPTION OF THE DRAWINGS

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings, of which:

FIG. 1 provides a non-limiting example of a plot showing the relationship between fragment length and origin of the fragment.

FIGS. 2A-2B provide non-limiting examples of flowcharts for a process for predicting an origin of an alteration in a sample from a patient, in accordance with embodiments of the present disclosure.

FIGS. 3A-3C provide non-limiting examples of features characterizing a selected plurality of reads, in accordance with embodiments of the present disclosure.

FIG. 4 provides a non-limiting example of a diagram for predicting an origin of an alteration in a sample from a patient, in accordance with embodiments of the present disclosure.

FIG. 5 provides a non-limiting example of a flowchart for a process for training a statistical model, in accordance with embodiments of the present disclosure.

FIG. 6 provides a non-limiting example of a diagram for training a statistical model, in accordance with embodiments of the present disclosure.

FIG. 7 provides a non-limiting example of a flowchart for obtaining training data to train a statistical model, in accordance with embodiments of the present disclosure.

FIG. 8 provides a non-limiting example of a diagram for obtaining training data to train a statistical model, in accordance with embodiments of the present disclosure.

FIG. 9 provides a non-limiting example of a diagram for training a statistical model, in accordance with embodiments of the present disclosure.

FIG. 10 provides a non-limiting example of a flowchart for obtaining training data to train a statistical model, in accordance with embodiments of the present disclosure.

FIG. 11 provides a non-limiting example of a flowchart for identifying a treatment for a patient, in accordance with embodiments of the present disclosure.

FIG. 12 provides a non-limiting example of a flowchart for identifying a monitoring requirement for a patient, in accordance with embodiments of the present disclosure.

FIG. 13 provides a non-limiting example of a flowchart for administering a treatment for a patient, in accordance with embodiments of the present disclosure.

FIG. 14 provides a non-limiting example of a flowchart for administering a monitoring type for a patient, in accordance with embodiments of the present disclosure.

FIG. 15 provides a non-limiting example of a flowchart for determining an adequacy of a patient sample for clinical decision-making, in accordance with embodiments of the present disclosure.

FIG. 16 provides a non-limiting example of a flowchart for determining one or more biomarkers, in accordance with embodiments of the present disclosure.

FIG. 17 depicts an exemplary computing device or system in accordance with embodiments of the present disclosure.

FIG. 18 depicts an exemplary computer system or computer network, in accordance with some instances of the systems described herein.

FIG. 19 depicts a plot showing the fragmentomic characteristics for germline, CH-derived, and tumor-derived fragments.

FIG. 20 depicts a plot showing the distribution of median fragment lengths for a tumor-derived sample and a CH-derived sample.

FIG. 21 depicts non-limiting examples of a ROC curve showing the accuracy of a prediction model, in accordance with embodiments of the present disclosure.

FIG. 22 depicts a non-limiting example of a plot showing the prediction score for a plurality of samples for alterations at different genes, in accordance with embodiments of the present disclosure.

FIG. 23A illustrates an ROC curve for an exemplary model trained in accordance with embodiments of the present disclosure.

FIG. 23B illustrates a plot that shows the overall prediction accuracy of the exemplary model, in accordance with embodiments of the present disclosure.

FIG. 23C illustrates a benchmark of methods for determining CH in accordance with embodiments of this disclosure compared to inferred CH determined via a computational CH prediction method.

### DETAILED DESCRIPTION

Methods, devices, and systems for predicting an origin of an alteration in a patient sample are described. Also described are methods for monitoring disease (e.g., cancer) in a patient based on the predicted origin of the alteration of interest in the patient sample. The disclosed methods have the potential to improve healthcare outcomes for patients (e.g., cancer patients) by providing healthcare providers with a test that can more accurately identify the presence of tumor-derived alterations in liquid biopsy samples. Embodiments in accordance with this disclosure can further improve the testing and monitoring of patients by ensuring that patient samples include a sufficient amount of tumor-derived variations to make a clinical determination. Embodiments in accordance with this disclosure can further improve the testing and monitoring of patients by improving the detection of biomarkers based on the patient sample.

Methods, devices, and systems for predicting an origin of an alteration in a sample from a patient are described. The prediction may be based on a score determined by a statistical model, such as a machine learning model. Also described are methods for selecting a treatment and/or monitoring a patient based on the prediction. The disclosed methods have the potential to improve healthcare outcomes for patients (e.g., cancer patients) by providing healthcare providers with better decision-making tools for detecting tumor-derived alterations in a patient sample.

In some instances, for example, methods for predicting an origin of an alteration in a sample from a patient comprise: receiving, using one or more processors, sequence read data obtained based on the sample; selecting, using the one or more processors, a plurality of reads from the sequence read data based on the alteration; determining, using the one or more processors, at least one feature characterizing the selected plurality of reads; inputting, using the one or more processors, the at least one feature into a statistical model, such as a trained machine learning model; generating, using the one or more processors, a score indicative of the origin of the alteration by the statistical model; and predicting, using the one or more processors, the origin of the alteration in the sample by comparing the score and one or more predefined thresholds.

In some instances, the at least one feature comprises at least one of: a fragment length for each of the selected plurality of reads, a start position of a fragment for the selected plurality of reads, and an end position of a fragment for the selected plurality of reads. In some instances, the at least one fragmentomic characteristic of the sample comprises at least one of: an amount of the selected plurality of reads having a specified length, a mean fragment length of the selected plurality of reads, a median fragment length of the selected plurality of reads, an interquartile range of the fragment lengths of the selected plurality of reads, a distribution of a fragment lengths of the selected plurality of reads, one or more peaks of a fragment lengths for the selected plurality of reads, a fragment length for at least one of the selected plurality of reads, a fragment end motif, a start position of a fragment for the selected plurality of reads, and an end position of a fragment for the selected plurality of reads. In some instances, the one or more additional features comprise an alteration depth, an allele frequency of the selected plurality of reads, an alteration coding type, a somatic determination, a germline determination, and a zygosity determination, a patient age, a blood tumor mutational burden score, a protein level data, a gene level data, an odds ratio describing an enrichment of the alteration in older patients over younger patients adjusted by disease group, a p-value describing a significance of an enrichment of the alteration in older patients over younger patients adjusted by disease group, an odds ratio describing an enrichment for a specific amino acid change in older patients over younger patients adjusted by disease group, a p-value describing a significance of an enrichment for a specific amino acid change in older patients over younger patients adjusted by disease group, an odds ratio describing an enrichment of the alteration in a certain cancer type compared to other cancers, a p-value describing a significance of an enrichment of the alteration in a certain cancer type compared to other cancers, an odds ratio describing an enrichment for a specific amino acid change in a certain cancer type compared to other cancers, a p-value describing a significance of an enrichment for a specific amino acid change in a certain cancer type compared to other cancers, a tumor fraction, an inferred subclonal status, a distribution of variant level germline, somatic, subclonal somatic calls, variant allele frequency changes over time, a sample level mutational signature score, a description of pathogenicity measures, or any combination thereof.

In some instances, methods for predicting an origin of an alteration in a sample from a patient comprise: receiving, using one or more processors, sequence read data associated with the sample; selecting, using the one or more processors, a plurality of reads from the sequence read data based on the alteration; determining, using the one or more processors, at least one fragmentomic characteristic based on the selected plurality of reads; inputting, using the one or more processors, the at least one fragmentomic characteristic into a statistical model; generating, using the one or more processors, a score indicative of the origin of the alteration by the statistical model; and predicting, using the one or more processors, the origin of the alteration in the sample by comparing the score and one or more predefined thresholds.

The disclosed methods and systems can improve the detection of tumor-derived alterations from liquid biopsy samples. For example, embodiments in accordance with this disclosure can further improve the testing and monitoring of patients by ensuring that patient samples include a sufficient amount of tumor-derived variations to make a clinical determination. Embodiments in accordance with this disclosure can further improve the testing and monitoring of patients by improving the detection of biomarkers based on the patient sample.

### Definitions

Unless otherwise defined, all of the technical terms used herein have the same meaning as commonly understood by one of ordinary skill in the art in the field to which this disclosure belongs.

As used in this specification and the appended claims, the singular forms “a”, “an”, and “the” include plural references unless the context clearly dictates otherwise. Any reference to “or” herein is intended to encompass “and/or” unless otherwise stated.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

As used herein, the terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain”), are inclusive or open-ended and do not exclude additional, un-recited additives, components, integers, elements, or method steps.

As used herein, the terms “individual,” “patient,” or “subject” are used interchangeably and refer to any single animal, e.g., a mammal (including such non-human animals as, for example, dogs, cats, horses, rabbits, zoo animals, cows, pigs, sheep, and non-human primates) for which treatment is desired. In particular embodiments, the individual, patient, or subject herein is a human.

The terms “cancer” and “tumor” are used interchangeably herein. These terms refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell, such as a leukemia cell. These terms include a solid tumor, a soft tissue tumor, or a metastatic lesion. As used herein, the term “cancer” includes premalignant, as well as malignant cancers.

As used herein, “treatment” (and grammatical variations thereof such as “treat” or “treating”) refers to clinical intervention (e.g., administration of an anti-cancer agent or anti-cancer therapy) in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

As used herein, the term “subgenomic interval” (or “subgenomic sequence interval”) refers to a portion of a genomic sequence.

As used herein, the term “subject interval” refers to a subgenomic interval or an expressed subgenomic interval (e.g., the transcribed sequence of a subgenomic interval).

As used herein, the terms “variant sequence” or “variant” are used interchangeably and refer to a modified nucleic acid sequence relative to a corresponding “normal” or “wild-type” sequence. In some instances, a variant sequence may be a “short variant sequence” (or “short variant”), i.e., a variant sequence of less than about 50 base pairs in length.

The terms “allele frequency” and “allele fraction” are used interchangeably herein and refer to the fraction of sequence reads corresponding to a particular allele relative to the total number of sequence reads for a genomic locus.

The terms “variant allele frequency” and “variant allele fraction” are used interchangeably herein and refer to the fraction of sequence reads corresponding to a particular variant allele relative to the total number of sequence reads for a genomic locus.

The term “fragmentomics” refers to any quantitative description of a cfDNA fragment including, but not limited to, a fragment length, a genomic site of origin of a cfDNA fragment, a sequence content a cfDNA fragment, a surrounding genomic context of a cfDNA fragment, a percentile of fragment lengths (e.g., a 25th percentile of fragment lengths, a 75th percentile of fragment lengths), a percentage of fragments less than or greater than a specific length threshold or a range of lengths threshold, or other quantitative feature of a cfDNA fragment. In one or more examples, the fragmentomic characteristics for the plurality of training sequencing reads can include at least one of an amount of a fragment having a specified length, a mean fragment length of the selected plurality of reads, a median fragment length of the selected plurality of reads, an interquartile range of fragment lengths of the selected plurality of reads, a distribution of fragment lengths of the selected plurality of reads, one or more peaks of fragment lengths for the selected plurality of reads, a fragment length for the selected plurality of reads, a start position of a fragment for the selected plurality of reads, and an end position of a fragment for the selected plurality of reads.

The term “statistical model” may include any trained or untrained model, and may include a machine learning model. The machine learning model can include an artificial intelligence (“AI”) learning model. The machine learning model can be at least one of a supervised model or an unsupervised model. In some instances, the statistical model can be a gradient boosting ensemble model. In some instances, the statistical model can be a convolutional neural network (CNN), an artificial neural network (ANN), a recurrent neural network (RNN), or other neural network. In some examples, the statistical model can be a Bayesian regression model, a random forest regression model, a support vector machine model, a linear regression model, a non-linear regression model, a multivariate regression machine learning model, a robust regression machine learning model, a neural network model, a nearest neighbor regression machine learning model, or a proportional hazards regression statistical model

The term “read length” may refer to a length of reads that is actually sequenced by a sequencing system or device.

The term “fragment length” may refer to a DNA fragment length that is specifically targeted for sequencing. The fragment length may be associated with certain biological processes. In some instances the fragment length may differ from the read length.

The section headings used herein are for organizational purposes only and are not to be construed as limiting the subject matter described.

### Methods for Determining an Origin of a Variation of Interest

Liquid biopsy tests provide healthcare providers with a less-invasive method of obtaining and analyzing patient sample for detecting potentially cancerous cells in order to diagnose and treat patient. Liquid biopsy tests examine cell free DNA (cfDNA) and can detect multiple categories of alterations, including germline alleles, somatic mutations from tumor cells, and alterations that drive clonal hematopoiesis (CH). Due to overlap in the underlying biological processes, the liquid biopsy tests can misclassify CH-derived alterations as tumor-derived alterations, which can inflate the number of tumor-derived alterations detected in the sample. A healthcare provider that recommends a therapy based on the results of a test that does not account for the potential misclassification of CH-derived alterations as tumor-derived alterations runs the risk of prescribing an ineffective treatment while exposing the patient to the risks and adverse side-effects associated with the treatment. Accordingly, healthcare providers and patients alike would benefit from having access to a more accurate cfDNA testing to improve clinical management for the patient's solid tumor.

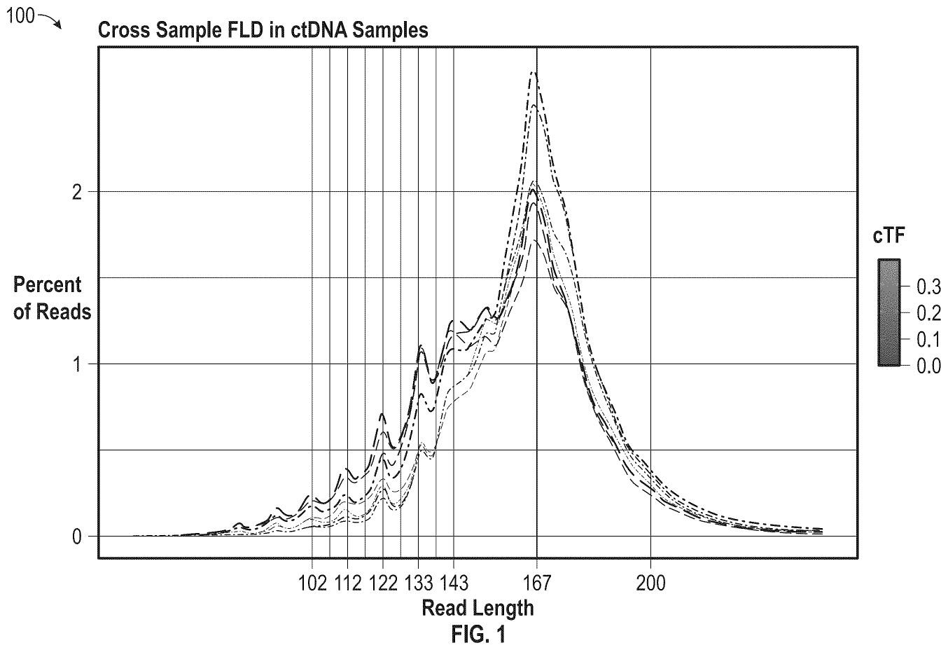

Embodiments of the present disclosure can determine an origin of an alteration in a sample. For example, systems and methods described herein can distinguish tumor-derived alterations from CH-derived alterations in liquid biopsy using genomic profiling data. In some instances, embodiments of the present disclosure can distinguish CH-derived alterations from tumor-derived alterations based on fragmentomic characteristics of cfDNA sequence reads from a sample. As an example of a fragmentomic characteristic of cfDNA sequence reads, the length distribution of cfDNA fragments is correlated with its cell of origin: cfDNA fragments arising from tumor cells tend to be shorter than cfDNA fragments from non-tumor cells. The inventors discovered that cfDNA fragments arising from tumor cells tend to be shorter than fragments arising from CH-derived alterations. As such, fragment length can be indicative of the origin of the cell (e.g., tumor-derived or CH-derived) for individual and groups of alterations. FIG. 1 is a plot 100 that illustrates the fragment size distribution for a range of samples, including samples having a high percentage of circulating tumor DNA (ctDNA) to samples having a low percentage of circulating tumor DNA (ctDNA). As shown in the figure, the samples with low ctDNA tend to have longer fragment length distributions, akin to healthy, tumor-free samples, compared to samples with high ctDNA. This demonstrates the concept that ctDNA has shorter fragment lengths than healthy-cell-derived cfDNA.

The disclosed methods for predicting the origin of one or more alterations of interest in a patient sample provide a number of potential advantages, by accounting for the presence of CH-derived alterations in a liquid biopsy sample, thereby enhancing the accuracy of an analysis of ctDNA presence in liquid biopsy samples and by reducing false positives of ctDNA. Embodiments in accordance with this disclosure can further improve the testing and monitoring of patients by ensuring that patient samples include a sufficient amount of tumor-derived variations to make a clinical determination. Embodiments in accordance with this disclosure can further improve the testing and monitoring of patients by improving the detection of biomarkers based on the patient sample.

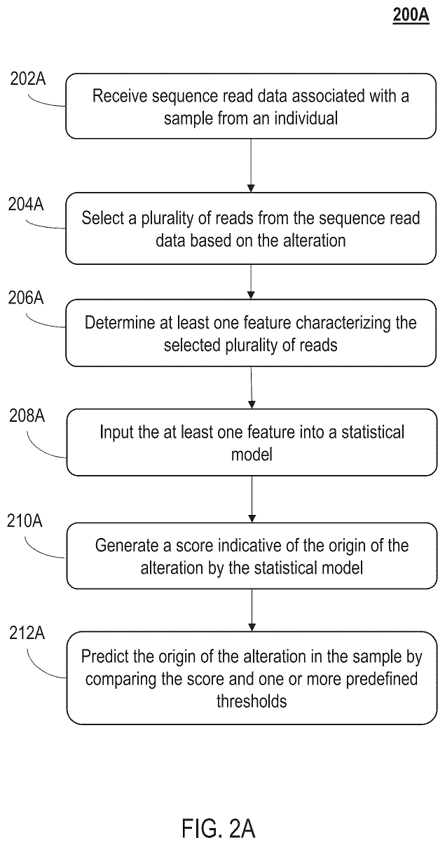

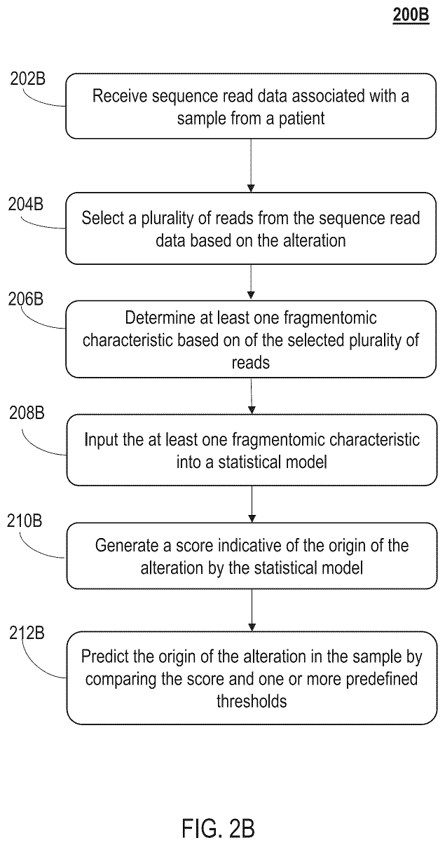

FIGS. 2A-2B provide non-limiting examples of flowcharts for processes 200A and 200B, respectively, for predicting an origin of an alteration of interest in a sample from a patient. Processes 200A and 200B can be performed, for example, using one or more electronic devices implementing a software platform. In some examples, processes 200A and 200B are performed using a client-server system, and the blocks of processes 200A and 200B are divided up in any manner between the server and a client device. In other examples, the blocks of processes 200A and 200B are divided up between the server and multiple client devices. Thus, while portions of processes 200A and 200B are described herein as being performed by particular devices of a client-server system, it will be appreciated that processes 200A and 200B are not so limited. In other examples, processes 200A and 200B are performed using only a client device or only multiple client devices. In processes 200A and 200B, some blocks are, optionally, combined, the order of some blocks is, optionally, changed, and some blocks are, optionally, omitted. In some examples, additional steps may be performed in combination with the processes 200A and 200B. Accordingly, the operations as illustrated (and described in greater detail below) are exemplary by nature and, as such, should not be viewed as limiting.

At step 202A in FIG. 2A, the system can receive sequence read data associated with a sample from the patient. In one or more examples, the sequence read data may be derived from sequencing circulating tumor DNA in a liquid biopsy sample. In one or more examples, the sequence read data may be received by the system as a BAM file.

In one or more examples, the sequence read data (derived from, e.g., targeted exome sequencing) include one or more short variants (SVs) in a patient sample.

In some instances, the sequence read data may be derived from, targeted exome sequencing or whole exome sequencing. In one or more examples, the whole exome sequencing can increase the number of genomic features (e.g., the number of short variants) detected.

In some instances, the sequence read data may also include other genomic features in addition to short variants, such as copy number alterations, rearrangements, chromosomal aneuploidy, whole genome doubling, Catalogue Of Somatic Mutations In Cancer (COSMIC) mutational signatures, or any combination thereof.

At step 204A in FIG. 2A, the system can select a plurality of reads from the sequence read data based on an alteration. According to an example, the system may use a pysam package to select sequencing reads from a consensus BAM file in a genomic region where the alteration resides. Sequence reads that include a wild type or a mutant sequence may be selected unless the reads are secondary reads, supplement reads, or a mapping quality of the reads is less than a threshold or a range of thresholds. Fragment length may be estimated based on the length of the sequence read, one or more locations the sequence reads on a reference genome, and an alignment of the sequence reads. In one or more examples, the plurality of reads may be selected based on a gene associated with the alteration. In one or more examples, the system can identify a plurality of reads from the sequence data that overlap with the alteration.

In one or more examples, the system can receive a particular alteration as an input from the user. The alteration can correspond to any alteration of interest in a sample. In one or more examples, the alteration can be determined based on one or more tests. In such examples, the system may predict the origin for any alteration identified in the test in accordance with the methods described herein. In one or more examples, the system can receive a plurality of alterations (e.g., a group of alterations), such that the system predicts an origin of the cells for each of the plurality of alterations.

In one or more examples, the alteration can correspond to one or more of an insertion, a deletion, and a substitution. In one or more examples, the alteration can correspond to alterations associated with clonal hematopoiesis. In one or more examples the alteration can include at least one of a simple or complex insertion, a simple or a complex deletion, or a base substitution. In one or more examples, the embodiments of this disclosure may apply to short variants, which can encompass any single nucleotide alterations. In one or more examples, a short variant can refer to a variant sequence of less than about 50 base pairs in length.

At step 206A in FIG. 2A, the system can determine at least one feature characterizing the selected plurality of reads. In one or more examples, the at least one feature can include a fragmentomic characteristic. For example, the at least one feature can include fragment lengths for the selected plurality of reads, a fragment end motif for the selected plurality of reads, a start position of the fragment for the selected plurality of reads and/or an end position of the fragment for the selected plurality of reads) or a combination thereof. The fragment length can refer to a length of a single strand of cfDNA, the size of which may be determined by extraction of the reference genome alignment coordinates of the corresponding paired-end sequence read of the selected plurality of reads. In one or more examples, the at least one feature can include one or more additional features characterizing the selected plurality of reads of the sample. The fragment end motif can refer to an expected sequence of a few nucleotides at fragment ends.

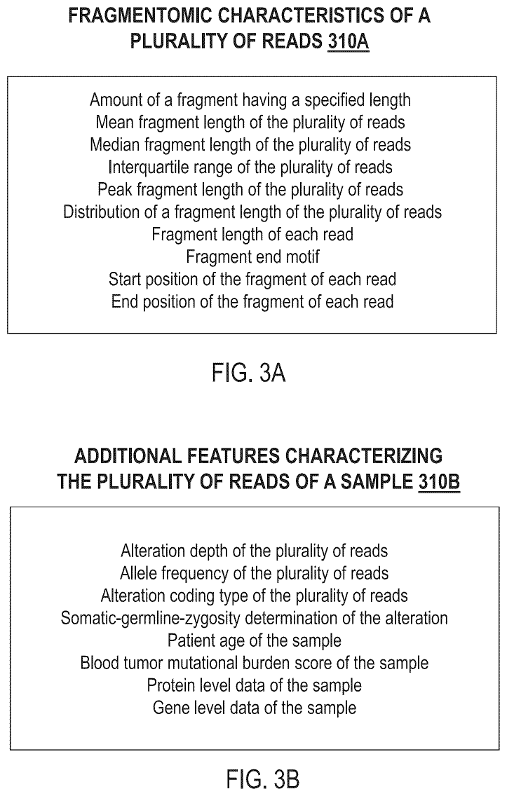

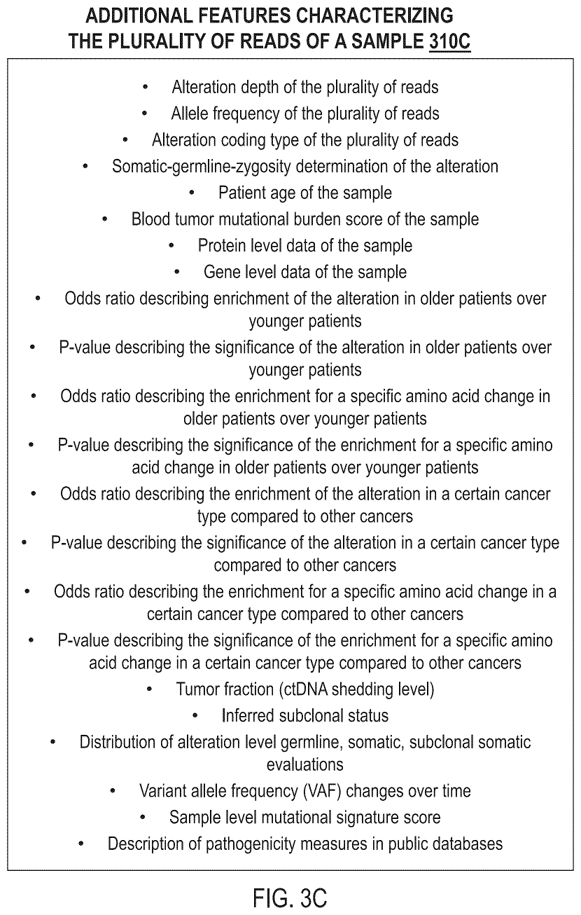

In one or more examples, the at least one feature characterizing the selected plurality of reads can correspond to one or more fragmentomic characteristics. FIG. 3A illustrates exemplary fragmentomic characteristics 310A of the selected plurality reads for a patient sample, according to one or more examples of the present disclosure. In one or more examples, exemplary fragmentomic characteristics 310A can include one or more of an amount of the selected plurality of reads having a specified length, a mean fragment length of the selected plurality of reads, a median fragment length of the selected plurality of reads, an interquartile range of fragment lengths of the selected plurality of reads, a distribution of fragment lengths of the selected plurality of reads, one or more peaks of fragment lengths for the selected plurality of reads, a fragment length for at least one of the selected plurality of reads, a fragment end motif, a start position of a fragment for the selected plurality of reads, and an end position of a fragment for the selected plurality of reads. The provided examples of fragmentomic characteristics 310A is not exhaustive and a skilled artisan would understand that additional fragmentomic characteristics could be determined for the plurality of reads without departing from the scope of this disclosure.

In some instances, the amount of a fragment having a specified length can correspond to a total amount (i.e., count) of fragments at the specified length. For example, for a particular sample, the total number of fragments that have a length below 100 bp, a length of 100 bp, a length of 101 bp, a length of 102 bp, . . . , a length of 550 bp, and a length greater than 550 bp. A skilled artisan will understand that these examples of specified lengths are exemplary and is not intended to limit the scope of the disclosure. For example, the specified length can correspond to a specific number of base pairs, a range of number of base pairs, or a combination thereof.

In some instances, the amount of a fragment having a specified length can correspond to a relative amount of fragments of a selected plurality of reads (e.g., reads overlapping with the alteration or gene of interest) corresponding to a specified length. In some examples, the amount of a fragment having a specified length can comprise a fraction. For example, the amount of a fragment having a specified length can be determined based on the number of fragments with a specified length (e.g., length below 100 bp, a length of 100 bp, a length of 101 bp, a length of 102 bp, . . . , a length of 550 bp, and a length greater than 550 bp) divided by the number of the selected plurality of reads to determine the amount of the fragments having a specified length. In some examples, the amount can correspond to a selected plurality of reads that include an alteration and/or a selected plurality of reads that include a wild type gene. A skilled artisan will understand that these examples of specified lengths are exemplary and is not intended to limit the scope of the disclosure.

In some instances, the mean fragment length of the selected plurality of reads can correspond to an average fragment length of the selected plurality of reads. In some instances, the median fragment length of the selected plurality of reads can correspond to the middle fragment length value of a sorted list of the fragment lengths of the selected plurality of reads. In some instances, the interquartile range of fragment lengths of the plurality of reads can correspond to a first fragment length value associated with the 25th percentile of the fragment lengths of the selected plurality of reads and a second fragment length value associated with the 75th percentile of the fragment lengths of the selected plurality of reads. In some instances, the peak fragment length can correspond to the mode or the fragment length value that appears most frequently in the length characteristics for the selected plurality of reads. In some instances, the system can determine more than one peak fragment length. In some instances, the distribution of the fragment length can correspond to a summary statistics characterizing the distribution, e.g., maximum value, minimum value, standard deviation, shape, etc.

In one or more examples, the system can determine the fragmentomic characteristics of the selected plurality of reads. In one or more examples, the statistical model can determine one or more fragmentomic characteristics of the selected plurality of reads based on length characteristics characterizing one or more of the selected plurality of reads.

In one or more examples, the system can also determine additional features characterizing the plurality of reads of a patient's sample. FIG. 3B illustrates exemplary additional features characterizing the plurality of reads 310B, according to one or more examples of the present disclosure. As shown in the figure, the exemplary additional features characterizing the plurality of reads 310B can include one or more of an alteration depth, an allele frequency, an alteration coding type, a somatic determination of the alteration, a germline determination of the alteration, and a zygosity determination, a somatic-germline-zygosity (SGZ) determination of the alteration, an age of patient providing the sample, a blood tumor mutational burden (bTMB) score of the sample, protein level data, and gene level data.

FIG. 3C illustrates exemplary additional features characterizing the plurality of reads 310C, according to one or more examples of the present disclosure. As shown in the figure, the exemplary additional features characterizing the plurality of reads 310C can include one or more of an alteration depth, an allele frequency, an alteration coding type, a somatic determination of the alteration, a germline determination of the alteration, and a zygosity determination, a somatic-germline-zygosity (SGZ) determination of the alteration, an age of patient providing the sample, a blood tumor mutational burden (bTMB) score of the sample, protein level data, and gene level data, an odds ratio describing the enrichment of the alteration in older patients over younger patients adjusted by disease group, a p-value describing the significance of the enrichment of the alteration in older patients over younger patients adjusted by disease group, an odds ratio describing the enrichment for a specific amino acid change in older patients over younger patients adjusted by disease group, a p-value describing the significance of the enrichment for a specific amino acid change in older patients over younger patients adjusted by disease group, an odds ratio describing the enrichment of the alteration in a certain cancer type compared to other cancers, a p-value describing the significance of the enrichment of the alteration in a certain cancer type compared to other cancers, an odds ratio describing the enrichment for a specific amino acid change in a certain cancer type compared to other cancers, a p-value describing the significance of the enrichment for a specific amino acid change in a certain cancer type compared to other cancers, an evaluation of ctDNA shedding level (i.e., tumor fraction), an inferred subclonal status (e.g., derived from comparison between VAF and tumor fraction), a distribution of variant level germline, somatic, subclonal somatic calls in silico, VAF changes over time when multiple plasma specimens are available, a sample level mutational signature score, a description of pathogenicity measures in public database (e.g. COSMIC mutational signatures). In some embodiments, the features can be determined with respect to one or more alterations in a certain gene.

In some instances, the alteration depth can correspond to a number of times that the locus corresponding to the alteration of interest was sequenced. In some instances, the alteration depth can correspond to a sequencing depth of the alteration. In some instances, the allele frequency can correspond to the relative frequency of an allele at a particular locus. In some instances, the alteration coding type can correspond to at least one of a missense, a nonsense, a frameshift, a non-frameshift, and the like. In some instances, the SGZ determination can correspond to a determination of whether the alteration is germline or somatic based on the allele frequency and allele specific copy number modeling. In one or more examples, the SGZ determination can be a computational SGZ determination. In some instances, the bTMB score can correspond to a measure of a mutational burden extrapolated from baited region of one or more sequenced reads. In some instances, the protein level data can include a measure of how frequently the alteration of the same protein effect in this gene are seen in liquid biopsy compared to tissue and a measure of how frequently alterations of the same protein effect in this gene are seen in older patients compared to younger patients. In some instances, the gene level data can include a measure of how frequently alterations in this gene are observed to be mutated in liquid biopsy compared to tissue and a measure of how frequently alterations in this gene are seen in older patients compared to younger patients. In some instances the age of the individual can correspond the age of the individual (e.g., patient or subject) from whom the sample was taken.

At step 208A in FIG. 2A, the system can input the at least one feature into a statistical model, such as a trained machine learning model. For example, the system can input one or more of the fragmentomic characteristics 310A or the additional feature characterizing the plurality of reads 310B into a trained machine learning model.

At step 210A in FIG. 2A, the system can generate a score indicative of the origin of the alteration by the statistical model. For example, the statistical model can be configured to generate a score indicative of whether the selected plurality reads are tumor-derived. In one or more examples, the score can be expressed as a percentage likelihood of whether the selected plurality of reads are tumor derived. In one or more examples, the score can be expressed as a percentage likelihood of whether the selected plurality of reads are not tumor derived, (e.g., CH-derived).

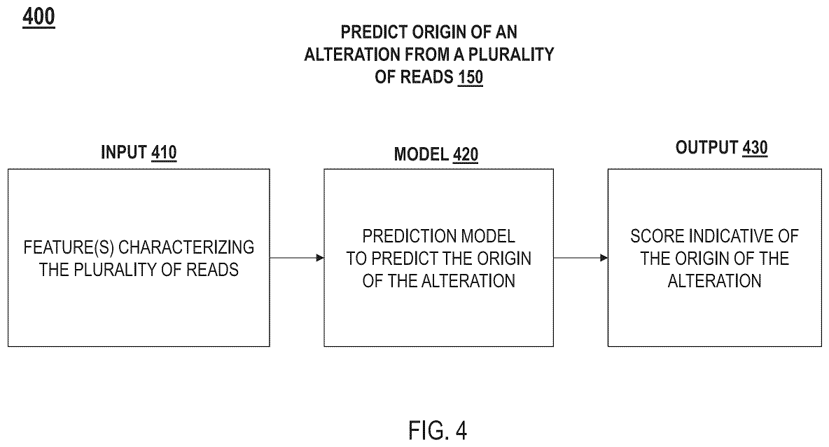

FIG. 4 is a diagram illustrating a process of predicting a score indicative of an origin of an alteration using a machine learning model, according to embodiments of the present disclosure. As shown in the figure, input data 410 corresponding to at least one feature characterizing the selected plurality of reads (e.g., fragmentomic characteristics 310A, or the additional feature characterizing the plurality of reads 310B) can be input into model 420. The model 420 can be a statistical model, such as a trained machine learning model configured to predict the origin of the alteration of interest. The model 420 can then output a score indicative of the origin of the alteration of interest 430. In one or more examples, model 420 can be associated with any of processes 200A and 200B.

In one or more examples, the input data 410 can include at least one fragmentomic characteristic 310A, and/or at least one additional feature characterizing the plurality of reads 310B. In such embodiments, the machine learning model 420 can be configured to extract additional fragmentomic characteristics from the input data 410. In this manner, the machine learning model can extract the fragmentomic characteristics 310B rather than manually extracting the fragmentomic characteristics from the length characteristics.

In some instances, the statistical model may be a trained model or an untrained model. In some instances, the statistical model can be a machine learning model. The machine learning model can include an artificial intelligence (“AI”) learning model. In some instances, the machine learning model can be at least one of a supervised model or an unsupervised model. In some instances the statistical model can be a gradient boosting ensemble model. In some instances, the statistical model can be a convolutional neural network (CNN), an artificial neural network (ANN), a recurrent neural network (RNN), or other neural network. In some examples, the statistical model can be a Bayesian regression model, a random forest regression model, a support vector machine model, a linear regression model, a non-linear regression model, a multivariate regression machine learning model, a robust regression machine learning model, a neural network model, a nearest neighbor regression machine learning model, or a proportional hazards regression statistical model.

In one or more examples, the model 420 can be trained to predict the origin of multiple alterations corresponding to different genes. For example, the model 420 can be trained based on input data 410 corresponding to a plurality of alterations. In one or more examples, the model 420 can be trained on a per gene basis, such that a single model is configured to predict the origin of the plurality of reads for a particular alteration. In such examples, input data corresponding to a particular alteration of interest can be input into the corresponding model 420 trained to predict the origin of the particular alteration of interest.

At step 212A in FIG. 2A, the system can predict the origin of the alteration in the sample by comparing the score (e.g., prediction score) and one or more predefined thresholds. For example, the system can compare the score to one or more predefined thresholds and determine whether the alteration is tumor-derived or CH-derived.

In one or more examples, a first threshold of the one or more thresholds can be determined such that if the score is above the threshold, then the plurality of reads are predicted to be not tumor-derived (e.g., CH-derived). In such examples, if the score is below the threshold, then the system can predict that the alteration is tumor derived. In one or more examples, if the score is below the threshold, then the system can indicate that the sequence read data of the plurality of reads is inconclusive. In such examples, the system may recommend that further testing (e.g., a normal paired test or an orthogonal test) should be completed to confirm the origin of the sequence read data of the plurality of reads.

In one or more examples, the one or more thresholds can include a first threshold and a second threshold lower than the first threshold. In such examples, if the score is above the first threshold, then the system can predict that the alteration is CH-derived. If the score is less than the second threshold, then the system can predict that the alteration is be tumor-derived. In one or more examples, if the score falls between the first threshold and the second threshold then the system can indicate that the sequence read data of the plurality of reads is inconclusive. In such examples, the system may recommend that further testing (e.g., a normal paired test or an orthogonal test) should be obtained to confirm the origin of the sequence read data of the plurality of reads.

In one or more examples, the one or more predetermined thresholds can be determined by maximizing or minimizing a function of sensitivity and specificity (such as the sum) For example, a loss function associated with performance metrics (e.g., whether the score corresponds to an accurate prediction) can be maximized or minimized. In some examples, the threshold can be set to maximize sensitivity and specificity.

In one or more examples, the one or more predetermined thresholds can be determined based on the area under the prediction function's receiver operating characteristic (ROC) curve. The area under a receiver operating characteristic curve can be used in statistics to measure the prediction accuracy of a binary classifier system. In one or more examples, the thresholds can be determined using one or more statistical techniques combined with predetermined confidence levels.

In one or more examples, each alteration (e.g., corresponding to a different gene) can be associated with different threshold levels. In one or more examples, each of alteration can be associated with the same threshold levels.

### Methods for Training a Model to Predict an Origin of an Alteration

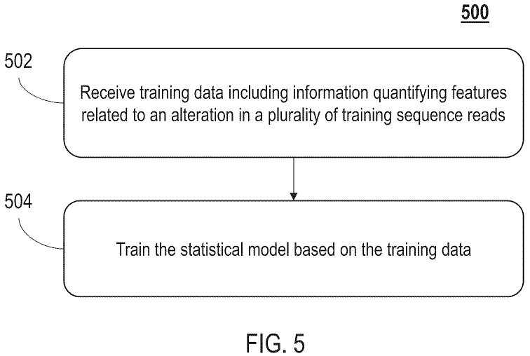

FIG. 5 provides a non-limiting example of a flowchart for a process 500 for training a machine learning model to predict an origin of an alteration of interest.

Process 500 can be performed, for example, using one or more electronic devices implementing a software platform. In some examples, process 500 is performed using a client-server system, and the blocks of process 500 are divided up in any manner between the server and a client device. In other examples, the blocks of process 500 are divided up between the server and multiple client devices. Thus, while portions of process 500 are described herein as being performed by particular devices of a client-server system, it will be appreciated that process 500 is not so limited. In other examples, process 500 is performed using only a client device or only multiple client devices. In process 500, some blocks are, optionally, combined, the order of some blocks is, optionally, changed, and some blocks are, optionally, omitted. In some examples, additional steps may be performed in combination with the process 500. Accordingly, the operations as illustrated (and described in greater detail below) are exemplary by nature and, as such, should not be viewed as limiting.

At step 502 in FIG. 5, the system can receive training data. In one or more examples, the training data can include information quantifying features related to an alteration in a plurality of training sequence reads. For example, the system can receive a plurality of subject samples that include quantifying features related to an alteration.

In one or more examples, the information quantifying features can include at least one of fragmentomic characteristics for each of a plurality of training sequencing reads, additional features for each of the plurality of training sequencing reads, and origin information for each of the plurality of training sequence reads.

In one or more examples, the fragmentomic characteristics for each of the plurality of training sequencing reads can include at least one of an amount of a fragment having a specified length, a mean fragment length of the selected plurality of reads, a median fragment length of the selected plurality of reads, an interquartile range of fragment lengths of the selected plurality of reads, a distribution of fragment lengths of the selected plurality of reads, one or more peaks of fragment lengths for the selected plurality of reads, a fragment length for the selected plurality of reads, a fragment end motif, a start position of a fragment for the selected plurality of reads, and an end position of a fragment for the selected plurality of reads, e.g., as described with respect to FIG. 3A.

In one or more examples, the additional features for each of the plurality of training sequencing reads can include at least one of an alteration depth, an allele frequency of the selected plurality of reads, an alteration coding type, a somatic determination, a germline determination, and a zygosity determination, a patient age, a blood tumor mutational burden score, a protein level data, or a gene level data.

In one or more examples, the origin information for each of the plurality of training sequence reads can correspond to a determination of whether the alteration from a subject's sample is tumor-derived or not tumor-derived based on a paired sample test.

At step 504 in FIG. 5, the system can train the machine-learning model based on the training data. The model can be trained to predict a score indicative of a likelihood that the plurality of reads are tumor derived.

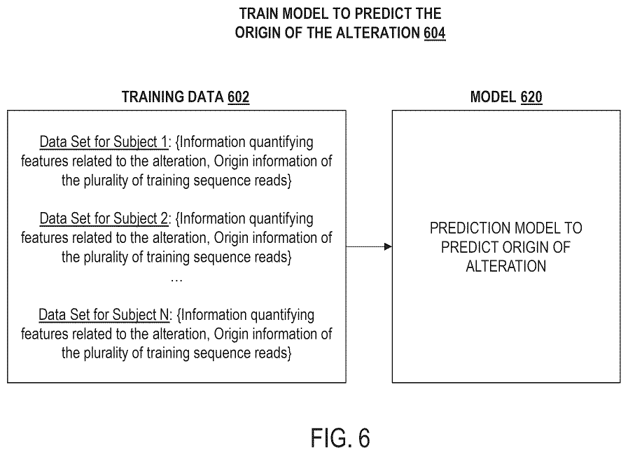

FIG. 6 illustrates a non-limiting example of a diagram for a process 600 for training a machine-learning model, according to embodiments of this disclosure. In one or more examples, process 600 can correspond to Step 504 of process 500. In one or more examples, the training at Step 504 can be applied to train model 420 described with respect to FIG. 4. As shown in FIG. 6, training data 602 can be input into model 620.

The training data 602 can include one or more data sets corresponding to a plurality of individuals (e.g., patients or subjects). Each data set can include input data associated with an alteration and a corresponding label indicative of an origin (e.g., CH-derived or tumor-derived) of the input data. For example, the input data can correspond to information quantifying features related to the alteration. In some instances, the information quantifying features related to the alteration can correspond to one or more fragmentomic characteristics of the plurality of training sequence reads (e.g., fragment end motifs, the mean fragment length, median fragment length, interquartile range values, and a peak fragment length of the plurality of training sequence reads, among others). In one or more examples, the input data can further include one or more additional features characterizing the plurality of training sequence reads (e.g., alteration depth, alteration coding type, a somatic determination of the alteration, a germline determination of the alteration, and a zygosity determination, patient age, BTMB score, protein level data, and gene level data). In some embodiments, the one or more fragmentomic characteristics may correspond to the fragmentomic characteristics described with respect to FIG. 3A. In some embodiments, the one or more additional features may correspond to the additional features described with respect to FIGS. 3B and 3C.

In one or more examples, the prediction score of the model, e.g., model 420, 620, can be determined based on a weighted evaluation of the input data (e.g., one or more fragmentomic characteristics of the plurality of training sequence reads and/or one or more additional features characterizing the plurality of training sequence reads and/or patient demographic information, such as age). For example, based on the training, the statistical model can assign weights to the input data to form a linear combination of the features and interaction of the features. An example formula can be Y=WX+b, where X is a vector of all features and complex interactions of the features, W is a matrix of weights, b is a vector of intercept, and Y is the linear predictor. Y is then transformed using a nonlinear function, such as the Sigmoid function into a probability within [0, 1], as the prediction score. In this manner, these weights may be based on data that has already been used to train the statistical model.

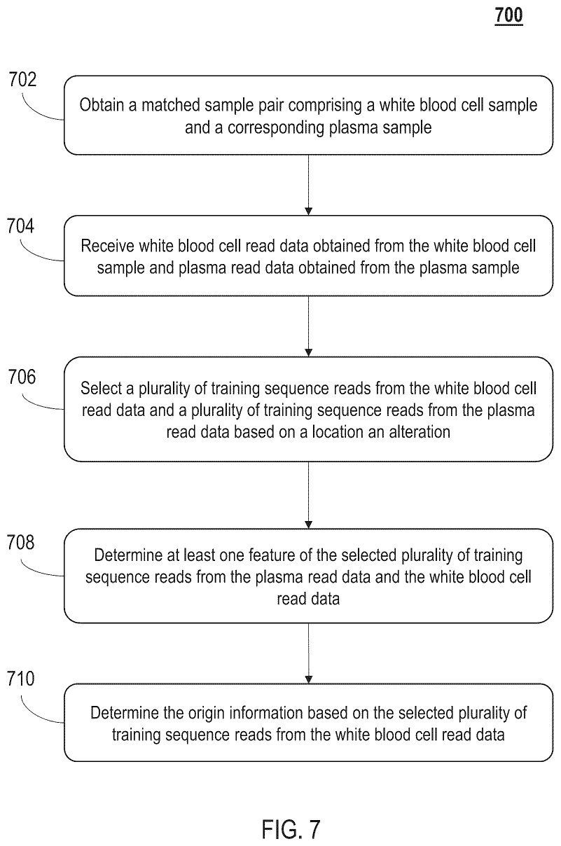

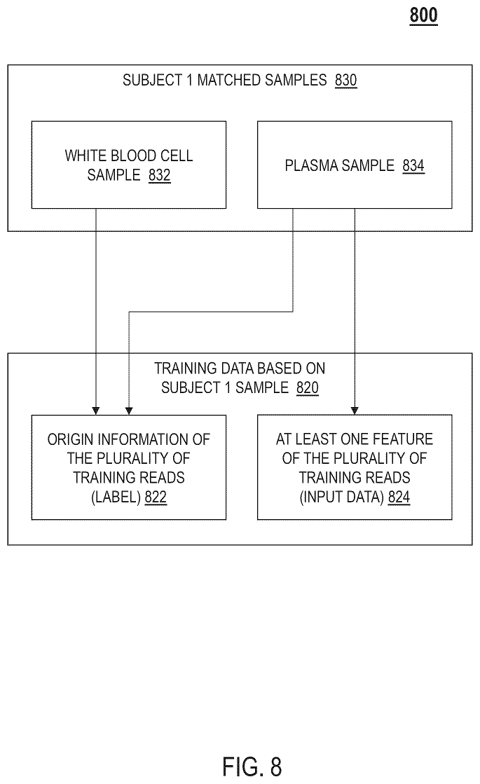

FIG. 7 provides a non-limiting example of a flowchart for a process 700 for obtaining training data according to embodiments of the present disclosure. FIG. 8 provides a non-limiting example of a diagram 800 for obtaining training data. As shown in diagram 800, in some instances, training data 820 can include origin information of the plurality of training sequence reads 822 and one or more features of the plurality of training sequence reads 824. In some examples, the origin information of the plurality of training sequence reads 822 can be used as a label, while the at least one feature of the plurality of training sequence reads 824 can be used as the input data for supervised learning. In some instances, this training data may be obtained based on a matched sample 830 from a subject (e.g., patient). The matched sample 830 can include a white blood cell sample 832, a plasma sample 834, or both.

Process 700 can be performed, for example, using one or more electronic devices implementing a software platform. In some examples, process 700 is performed using a client-server system, and the blocks of process 700 are divided up in any manner between the server and a client device. In other examples, the blocks of process 700 are divided up between the server and multiple client devices. Thus, while portions of process 700 are described herein as being performed by particular devices of a client-server system, it will be appreciated that process 700 is not so limited. In other examples, process 700 is performed using only a client device or only multiple client devices. In process 700, some blocks are, optionally, combined, the order of some blocks is, optionally, changed, and some blocks are, optionally, omitted. In some examples, additional steps may be performed in combination with the process 700. Accordingly, the operations as illustrated (and described in greater detail below) are exemplary by nature and, as such, should not be viewed as limiting. The following description of process 700 will be made with reference to diagram 800.

At step 702 in FIG. 7, the system can obtain a matched sample pair comprising a white blood cell sample and a corresponding plasma sample. For example, the system can obtain a set of matched samples 830 from a first subject (e.g., subject 1). As shown in diagram 800 the set of matched samples 830 can include a white blood cell sample 832 and a plasma sample 834.

At step 704 in FIG. 7, the system can receive white blood cell read data obtained from the white blood cell sample and plasma read data obtained from the plasma sample. In one or more examples, the white blood cell read data and the plasma read data can include one or more short variants (SVs). In one or more examples, white blood cell read data and the plasma read data may be received by the system as BAM files.

In some instances, the white blood cell read data and the plasma read data may also include other genomic features in addition to short variants, such as copy number alterations, rearrangements, chromosomal aneuploidy, whole genome doubling, Catalogue Of Somatic Mutations In Cancer (COSMIC) mutational signatures, or any combination thereof.

In some instances, the white blood cell read data and the plasma read data may be derived from, targeted exome sequencing or whole exome sequencing. In one or more examples, the whole exome sequencing can increase the number of genomic features (e.g., the number of short variants) detected.

At step 706 in FIG. 7, the system can select the plurality of training sequence reads from the white blood cell read data based on the location of the alteration and further select a plurality of training sequence reads from the plasma read data based on a location of the alteration. In one or more examples, the system can identify a plurality of training sequence reads from the sequence data that overlap with the alteration in the white blood cell read data and the alteration in the plasma read data.

At step 708 in FIG. 7, the system can determine at least one feature of the plurality of training sequence reads 824 from the plasma read data and the white blood cell read data. In one or more examples, the at least one feature of the selected plurality of training sequence reads 824 can include sequence read length characteristics (e.g., fragment length, fragment end motif, fragment start position, fragment end position, etc.) one or more fragmentomic characteristics of the plurality of training sequence reads, and one or more additional features characterizing the plurality of sequencing reads (e.g. alteration depth, alteration coding type, a somatic determination of the alteration, a germline determination of the alteration, and a zygosity determination, patient age, BTMB score, protein level data, and gene level data). In some embodiments, the one or more fragmentomic characteristics may correspond to the fragmentomic characteristics described with respect to FIG. 3A. In some embodiments, the one or more additional features may correspond to the additional features described with respect to FIGS. 3B and 3C.

For example, the system can extract one or more length characteristics associated with each read of the plurality of training sequence reads from the plasma read data of the plasma sample 834 and the white blood cell read data of the white blood cell sample 832. In some instances, the one or more length characteristics can include a fragment length, a start location of a read, and an end location of a read.

The system can further determine one or more additional fragmentomic characteristics of the selected plurality of training reads based on the extracted length characteristics, as discussed above. For example, the fragmentomic characteristics can include, but is not limited to, one or more of a mean fragment length of the selected plurality of reads, a median fragment length of the selected plurality of reads, an interquartile range of the selected plurality of reads, and a peak fragment length or a mode fragment length of the selected plurality of reads.

In one or more examples, the system can also determine additional features characterizing the plurality of training reads of the plasma sample 834 and the white blood sample 832. The additional features can correspond to alteration features (e.g., an alteration depth, an alteration coding type, a somatic determination of the alteration, a germline determination of the alteration, and a zygosity determination), sample features (e.g., a patient age of the sample and a blood tumor mutational burden (BTMB), an allele frequency, protein level data and gene level data). A skilled artisan will understand that these exemplary features are not intended to be exhaustive and more or less features can be used to characterize the plurality of training sequence reads of the plasma sample and the white blood sample without departing from the scope of this disclosure.

At step 710 in FIG. 7, the system can determine the origin information based on the selected plurality of training sequence reads from the white blood cell read data and the plasma sample read data. The origin information can correspond to the plurality of training sequence reads being tumor-derived or not tumor-derived. For example, if the system detects an alteration of interest in both the plurality of training sequence reads from the white blood cell red data and the plurality of training sequence reads from the plasma read data, then the system determines that the alteration of interest is CH-derived. If the system detects an alteration of interest in the plurality of training sequence reads from the plasma read data, then the system determines that the alteration of interest is tumor-derived. In one or more examples, both false negatives and false positives may be controlled by a matched assay process including, but not limited to centralized lab, comparable sequencing depth, QC control, and the like. In some examples, false positives may occur when white blood cell sequencing picks up ctDNA signals, which may be filtered by a threshold of ratio between plasma allele frequency and peripheral blood mononuclear cell (PBMC) allele frequency. In such scenarios, the false positives may have a clearly inflated ratio. The system can then label the corresponding at least one feature of the plurality of training sequence reads 824 determined in Step 708 with the appropriate label.

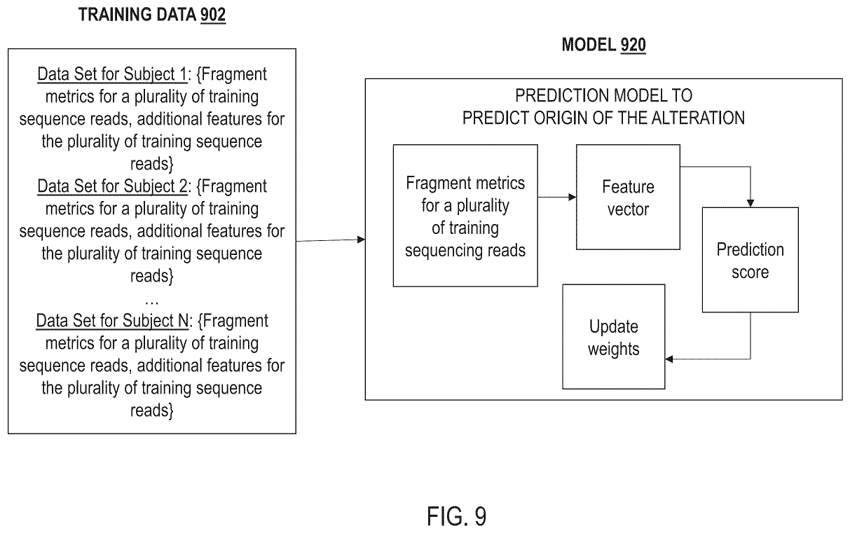

FIG. 9 provides a non-limiting example of a flowchart for a process 900 for training a machine-learning model, according to embodiments of this disclosure. In one or more examples, process 900 can correspond to Step 504 of process 500. In one or more examples, the training at Step 504 can be applied to train model 420 described with respect to FIG. 4. As shown in FIG. 9, training data 902 can be input into model 920 for training.

The training data 902 can include one or more data sets each corresponding to a subject (e.g., patient). The training data 902 can include input data including fragment metrics for a plurality of training sequence reads. In one or more examples, model 920 can be an unsupervised model. In one or more examples, model 920 can be a neural network model (e.g., convolutional neural network model).

In some instances, the fragment metrics for a plurality of training sequence reads can correspond to one or more of the fragmentomic characteristics of a plurality of reads 310A. In some examples, the fragment metrics can include amounts corresponding to fragments having a specified length. In some instances, one or more fragment metrics can be expressed as a count corresponding to fragments having a specified length as described above. In some instances, the fragment metric can be expressed in relation with the selected plurality of reads (e.g., expressed as a fraction), as described above. For example, the amount of a fragment having a specified length can be determined based on the number of fragments with a specified length (e.g., length below 100 bp, a length of 100 bp, a length of 101 bp, a length of 102 bp, . . . , a length of 550 bp, and a length greater than 550 bp) divided by the number of the selected plurality of reads to determine the amount of the fragments having a specified length. In some examples, the amount can correspond to a selected plurality of reads that include an alteration and/or a selected plurality of reads that include a wild type gene. A skilled artisan will understand that these examples of specified lengths are exemplary and is not intended to limit the scope of the disclosure. In some examples, the fragment metrics can include a mean fragment length of the selected plurality of reads, a median fragment length of the selected plurality of reads, an interquartile range of fragment lengths of the selected plurality of reads, a distribution of fragment lengths of the selected plurality of reads, one or more peaks of fragment lengths for the selected plurality of reads, a fragment length for at least one of the selected plurality of reads, a fragment end motif, a start position of a fragment for the selected plurality of reads, and an end position of a fragment for the selected plurality of reads. In some embodiments, the fragment metrics can be stored in an array (e.g., two-dimensional array) to be used as an input into the model 920.

As shown in the figure, during training, the model 920 can receive the training data 902 (e.g., corresponding to fragment metrics for a plurality of training sequencing reads). Based on the training data 902, the model 920 can extract a feature vector for each data set (e.g., for each array). The model 920 can then determine the prediction score based on the feature vector. In some instances, the model 920 can update weights based on the prediction score.

In one or more examples, the model 920 can be a convolutional neural network including one or more convolution layers, one or more pooling layers, and/or one or more dropout layers. These layers can extract a feature vector from the 2D array. In such embodiments, the extracted feature vector may be concatenated with one or more additional features (e.g., a patient age of the sample and a blood tumor mutational burden (BTMB), an allele frequency, protein level data and gene level data). In some instances, these additional features may correspond to one or more features described with respect to FIGS. 3B and 3C. In some instances, one or more dense layers of a deep neural network may use the concatenated feature vector to determine the probability score as output. In some instances, the CNN model parameters may be optimized by minimizing a binary cross-entropy loss function using a stochastic gradient descent optimization algorithm. Accordingly, in such embodiments, the model can be configured to extract features from fragment sizes, rather than manually extracting features. In one or more examples, the model training can use Keras and/or TensorFlow platforms.

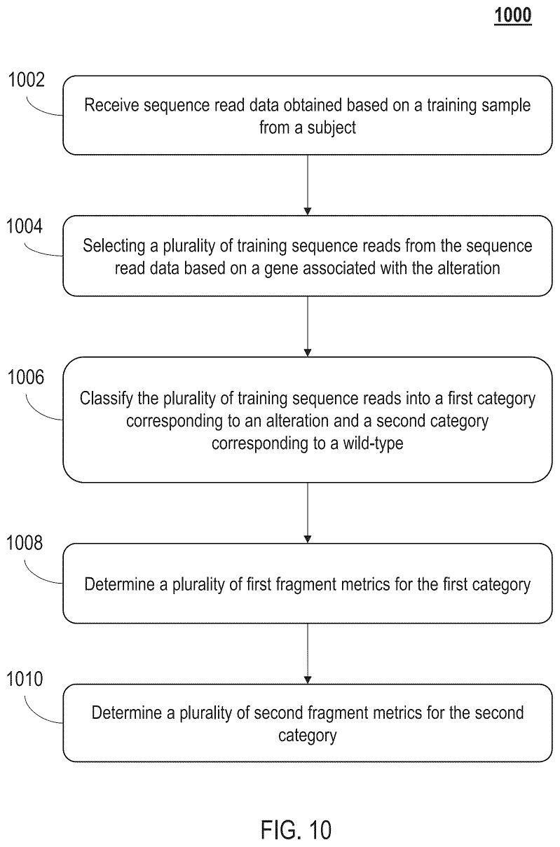

FIG. 10 provides a non-limiting example of a flowchart for a process 1000 for obtaining training data according to embodiments of the present disclosure.

Process 1000 can be performed, for example, using one or more electronic devices implementing a software platform. In some examples, process 1000 is performed using a client-server system, and the blocks of process 1000 are divided up in any manner between the server and a client device. In other examples, the blocks of process 1000 are divided up between the server and multiple client devices. Thus, while portions of process 1000 are described herein as being performed by particular devices of a client-server system, it will be appreciated that process 1000 is not so limited. In other examples, process 1000 is performed using only a client device or only multiple client devices. In process 1000, some blocks are, optionally, combined, the order of some blocks is, optionally, changed, and some blocks are, optionally, omitted. In some examples, additional steps may be performed in combination with the process 1000. Accordingly, the operations as illustrated (and described in greater detail below) are exemplary by nature and, as such, should not be viewed as limiting.

At step 1002 in FIG. 10, the system can receive sequence read data associated with a training sample from a subject. In one or more examples, the training sample can correspond to a liquid biopsy sample.

At step 1004 in FIG. 10, the system can select a plurality of training sequence reads from the sequence read data based on a gene associated with the alteration. For example, the plurality of training sequence reads can correspond to the alteration as well as the wild-type. In one or more examples, the selected plurality of training sequence reads may overlap the alteration and/or gene associated with the alteration. In some embodiments, the selected plurality of training sequence reads may also pass quality controls. For example, the training reads may have a mapping quality greater than 26, the training reads may be primary mapping reads, and the training reads may be representative alignments, among others. In one or more examples, the training reads may have a mapping quality between 10-20, 20-30, 30-40, 40-50, or a combination thereof.

At step 1006 in FIG. 10, the system can classify the plurality of training sequence reads into a first category corresponding to the alteration and a second category corresponding to a wild-type.

At step 1008 in FIG. 10, the system can determine a plurality of first fragment metrics for the first category. In some instances, the fragment metrics may correspond to a total amount of fragments at a specified length, fragment end motifs, and/or a relative amount of fragments at a specified length relative to the other selected plurality of reads. As discussed above, the relative amount of fragments at a specified length can be determined by, for example, determining the number of fragments with a specified length (e.g., a length below 100 bp, 100 bp, 101 bp, 102 bp, . . . , 550 bp, above 550 bp) and dividing this number by the total number of fragments in the first category. The exemplary fragment lengths provided are exemplary and other fragment lengths and/or ranges of fragment lengths can be used without departing from the scope of this disclosure. At step 1010 in FIG. 10, the system can determine a plurality of second fragment metrics for the second category. Step 1010 may be determined with respect to the second category in a manner similar to step 1006.

In one or more examples, the plurality of first fragment metrics and the plurality of second fragment metrics can be stored in a two dimensional array. In one or more examples, the two dimensional array can correspond to the training data 902 for the data set for a subject as described above.

In one or more examples, the fragmentomic characteristics (e.g., fragmentomic characteristics encoded in the two-dimensional array) can be processed with a convolutional neural network (CNN). In one or more examples, the CNN can include one or more one or more convolution layers, one or more pooling layers, one or more dropout layers, one or more dense layers, or a combination thereof. In such examples, the output the CNN can be concatenated with additional, non-fragmentomic features. For example, the output of the CNN may be an output vector that can be concatenated with non-fragmentomic features such as alteration depth, an allele frequency of the selected plurality of reads, an alteration coding type, a somatic determination, a germline determination, and a zygosity determination, a patient age, a blood tumor mutational burden score, a protein level data, a gene level data, or any combination thereof. The concatenated feature vector may be input into a deep neural network to output a prediction score. In some examples, the prediction score can correspond to a probability between 0 and 1.

FIG. 2B provides a non-limiting example of a flow-chart for predicting an origin of an alteration of interest in a sample from a patient. At step 202B in FIG. 2B, the system can receive sequence read data associated with a sample from the patient. In one or more examples, the sequence read data may be derived from sequencing circulating tumor DNA in a liquid biopsy sample. In one or more examples, the sequence read data may be received by the system as a BAM file. In some examples, step 202B may be substantially similar to step 202A described above with respect to FIG. 2A.

At step 204B in FIG. 2B, the system can select a plurality of reads from the sequence read data based on an alteration. In one or more examples, the plurality of reads may be selected based on a gene associated with the alteration. In one or more examples, the system can identify a plurality of reads from the sequence data that overlap with the alteration. In some examples, step 204B may be substantially similar to step 204A described above with respect to FIG. 2A.

At step 206B in FIG. 2B, the system can determine at least one fragmentomic characteristic characterizing the selected plurality of reads. For example, as discussed above, the at least one fragmentomic characteristic can include fragment lengths for the selected plurality of reads, an amount of fragment having a specified length, a mean fragment length of the selected plurality of reads, a median fragment length of the selected plurality of reads, an interquartile range of fragment lengths of the selected plurality of reads, a distribution of fragment lengths of the selected plurality of reads, one or more peaks of fragment lengths for the selected plurality of reads, a fragment end motif for the selected plurality of reads, a start position of the fragment for the selected plurality of reads, an end position of the fragment for the selected plurality of reads, or a combination thereof. The fragmentomic characteristics may correspond to the fragmentomic characteristics described with respect to FIG. 3A. In one or more examples, additional features associated with the selected plurality of reads of the sample can be input into the statistical model. The additional features associated with the selected plurality of reads may correspond to the characteristics described with respect to FIGS. 3B and 3C.

In one or more examples, the system can determine the fragmentomic characteristics of the selected plurality of reads. In one or more examples, the statistical model can determine one or more fragmentomic characteristics of the selected plurality of reads based on length characteristics or fragment end motifs characterizing one or more of the selected plurality of reads.

At step 208B in FIG. 2B, the system can input the at least one fragmentomic characteristic into a statistical model, such as a trained machine learning model. For example, the system can input one or more of the fragmentomic characteristics 310A or the additional feature(s) characterizing the plurality of reads 310B into a trained machine learning model. In some examples, step 208B may be substantially similar to step 208A described above with respect to FIG. 2A.

At step 210B in FIG. 2B, the system can generate a score indicative of the origin of the alteration by the statistical model. For example, the statistical model can be configured to generate a score indicative of whether the selected plurality reads are tumor-derived. In one or more examples, the score can be expressed as a percentage likelihood of whether the selected plurality of reads are tumor derived. In one or more examples, the score can be expressed as a percentage likelihood of whether the selected plurality of reads are not tumor derived, (e.g., CH-derived). In some examples, step 210B may be substantially similar to step 210A described above with respect to FIG. 2A.

At step 212B in FIG. 2B, the system can predict the origin of the alteration in the sample by comparing the score (e.g., prediction score) and one or more predefined thresholds. For example, the system can compare the score to one or more predefined thresholds and determine whether the alteration is tumor-derived or CH-derived. In some examples, step 212B may be substantially similar to step 212A described above with respect to FIG. 2A.

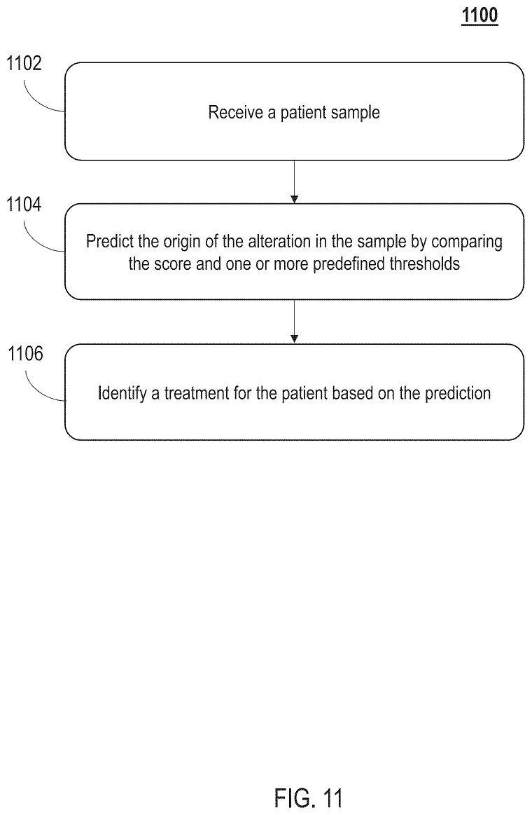

FIG. 11 provides a non-limiting example of a flowchart for a process 1100 for identifying a treatment for a patient according to embodiments of the present disclosure.

Process 1100 can be performed, for example, using one or more electronic devices implementing a software platform. In some examples, process 1100 is performed using a client-server system, and the blocks of process 1100 are divided up in any manner between the server and a client device. In other examples, the blocks of process 1100 are divided up between the server and multiple client devices. Thus, while portions of process 1100 are described herein as being performed by particular devices of a client-server system, it will be appreciated that process 1100 is not so limited. In other examples, process 1100 is performed using only a client device or only multiple client devices. In process 1100, some blocks are, optionally, combined, the order of some blocks is, optionally, changed, and some blocks are, optionally, omitted. In some examples, additional steps may be performed in combination with the process 1100. Accordingly, the operations as illustrated (and described in greater detail below) are exemplary by nature and, as such, should not be viewed as limiting.

At step 1102 in FIG. 11, the system can receive a patient sample. In some instances, the patient sample can correspond to a liquid biopsy sample taken from a patient.

At step 1104 in FIG. 11, the system can predict the origin of the alteration in the sample by comparing the score and one or more predefined thresholds. In one or more examples, step 1104 can correspond to any of processes 200A and 200B described above.

At step 1106 in FIG. 11, the system can identify a treatment for the patient based on the prediction. For example, if the prediction indicates that the alteration is tumor-derived, the system can identify an appropriate treatment for the patient. In such examples, because the alteration is determined to be tumor-derived, the system may determine that the treatment will be able to effectively treat the disease. In some instances, if the system predicts that the alteration of interest is not tumor-derived (e.g., that the results are inconclusive), the system can may not identify a treatment for the patient because such a treatment would not effectively treat the disease (e.g., because the alterations are not tumor derived). In such instances, the system may recommend that further monitoring of the patient and/or testing be conducted.

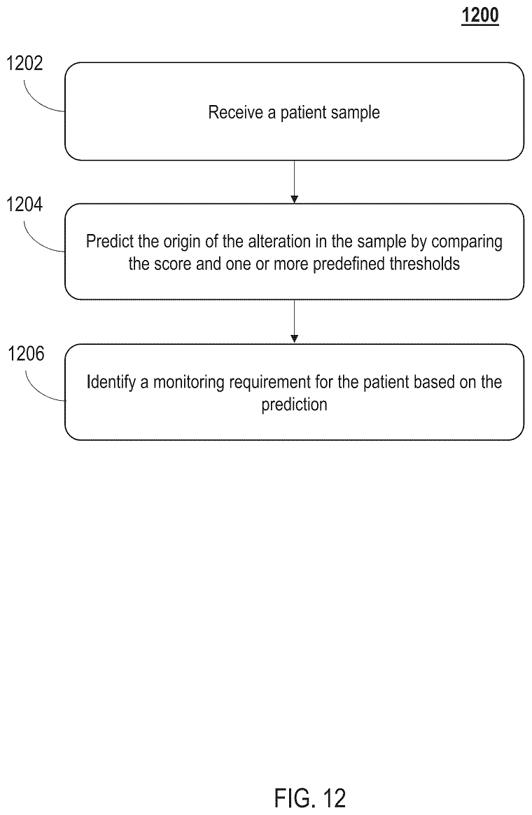

FIG. 12 provides a non-limiting example of a flowchart for a process 1200 for identifying a monitoring requirement for a patient based on the prediction according to embodiments of the present disclosure.

Process 1200 can be performed, for example, using one or more electronic devices implementing a software platform. In some examples, process 1200 is performed using a client-server system, and the blocks of process 1200 are divided up in any manner between the server and a client device. In other examples, the blocks of process 1200 are divided up between the server and multiple client devices. Thus, while portions of process 1200 are described herein as being performed by particular devices of a client-server system, it will be appreciated that process 1200 is not so limited. In other examples, process 1200 is performed using only a client device or only multiple client devices. In process 1200, some blocks are, optionally, combined, the order of some blocks is, optionally, changed, and some blocks are, optionally, omitted. In some examples, additional steps may be performed in combination with the process 1200. Accordingly, the operations as illustrated (and described in greater detail below) are exemplary by nature and, as such, should not be viewed as limiting.

At step 1202 in FIG. 12, the system can receive a patient sample. In some instances the patient sample can correspond to a liquid biopsy sample taken from a patient.

At step 1204 in FIG. 12, the system can predict the origin of the alteration in the sample by comparing the score and one or more predefined thresholds. In one or more examples, step 1204 can correspond to any of processes 200A and 200B described above.

At step 1206 in FIG. 12, the system can identify a monitoring requirement for the patient based on the prediction, according to embodiments of this disclosure. In some instances, monitoring may be recommended if the system predicts that the alteration is tumor derived. In some instances, monitoring may be recommended if the system predicts that the alteration is CH-derived. As discussed above, in some instances, the system may recommend that further monitoring should be conducted. In some instances, the further monitoring can include obtaining additional samples from a patient. In such examples, the system may determine an origin of the alteration in the additional sample using one or more tests. In some examples, the one or more tests may differ from the prediction model, e.g., prediction model 420, 620. In some examples, the one or more tests may include a paired normal test (e.g., sequencing blood mononuclear cells), an orthogonal test, and the like. In one or more examples, the monitoring requirement may include reflex tissue testing.

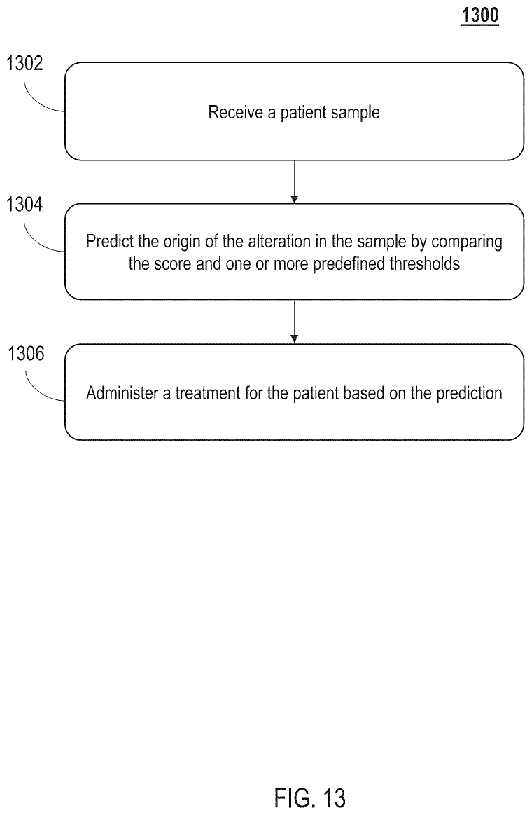

FIG. 13 provides a non-limiting example of a flowchart for a process 1300 for identifying a treatment for a patient according to embodiments of the present disclosure.

Process 1300 can be performed, for example, using one or more electronic devices implementing a software platform. In some examples, process 1300 is performed using a client-server system, and the blocks of process 1300 are divided up in any manner between the server and a client device. In other examples, the blocks of process 1300 are divided up between the server and multiple client devices. Thus, while portions of process 1300 are described herein as being performed by particular devices of a client-server system, it will be appreciated that process 1300 is not so limited. In other examples, process 1300 is performed using only a client device or only multiple client devices. In process 1300, some blocks are, optionally, combined, the order of some blocks is, optionally, changed, and some blocks are, optionally, omitted. In some examples, additional steps may be performed in combination with the process 1300. Accordingly, the operations as illustrated (and described in greater detail below) are exemplary by nature and, as such, should not be viewed as limiting.

At step 1302 in FIG. 13, the system can receive a patient sample. In some instances the patient sample can correspond to a liquid biopsy sample taken from a patient.

At step 1304 in FIG. 13, the system can predict the origin of the alteration in the sample by comparing the score and one or more predefined thresholds. In one or more examples, step 1304 can correspond to any of processes 200A and 200B described above.

At step 1306 in FIG. 13, the system can administer a treatment for the patient based on the prediction. For example, if the prediction indicates that the alteration is tumor-derived, the system can administer an appropriate treatment for the patient. In such examples, because the alteration is determined to be tumor-derived, the system may determine that the treatment will be able to effectively treat the disease. In some instances, if the system predicts that the alteration of interest is not tumor-derived (e.g., that the results are inconclusive), the system can may not administer a treatment for the patient because such a treatment would not effectively treat the disease (e.g., because the alterations are not tumor derived). In such instances, the system may instead recommend that further monitoring of the patient and/or testing be conducted.

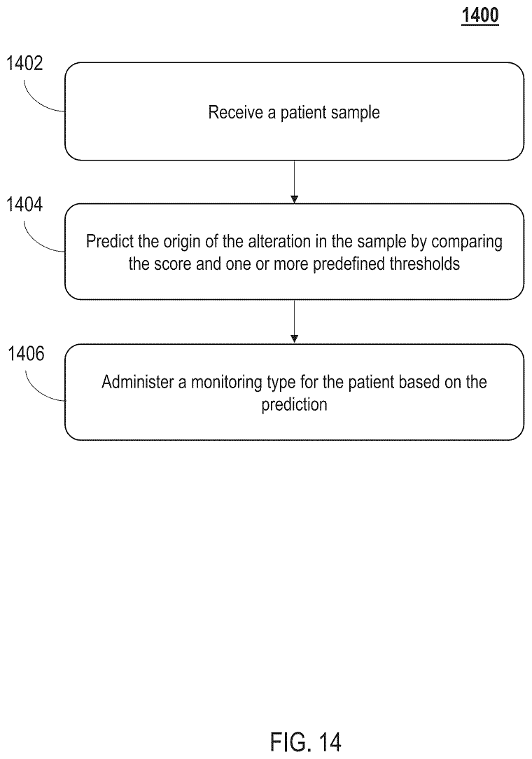

FIG. 14 provides a non-limiting example of a flowchart for a process 1400 for identifying a monitoring requirement for a patient based on the prediction according to embodiments of the present disclosure.

Process 1400 can be performed, for example, using one or more electronic devices implementing a software platform. In some examples, process 1400 is performed using a client-server system, and the blocks of process 1400 are divided up in any manner between the server and a client device. In other examples, the blocks of process 1400 are divided up between the server and multiple client devices. Thus, while portions of process 1400 are described herein as being performed by particular devices of a client-server system, it will be appreciated that process 1400 is not so limited. In other examples, process 1400 is performed using only a client device or only multiple client devices. In process 1400, some blocks are, optionally, combined, the order of some blocks is, optionally, changed, and some blocks are, optionally, omitted. In some examples, additional steps may be performed in combination with the process 1400. Accordingly, the operations as illustrated (and described in greater detail below) are exemplary by nature and, as such, should not be viewed as limiting.

At step 1402 in FIG. 14, the system can receive a patient sample. In some instances the patient sample can correspond to a liquid biopsy sample taken from a patient.

At step 1404 in FIG. 14, the system can predict the origin of the alteration in the sample by comparing the score and one or more predefined thresholds. In one or more examples, step 1404 can correspond to any of processes 200A and 200B described above.

At step 1406 in FIG. 14, the system can administer a monitoring type for the patient based on the prediction, according to embodiments of this disclosure. As discussed above, in some instances, the system may recommend that further monitoring should be conducted. In some instances, the monitoring type can include obtaining additional samples from a patient. In such examples, the system may determine an origin of the alteration in the additional sample using one or more tests. In some examples, the one or more tests may differ from the prediction model, e.g., prediction model 420, 620. In some examples, the one or more tests may include a paired normal test (e.g., sequencing blood mononuclear cells), an orthogonal test, and the like.

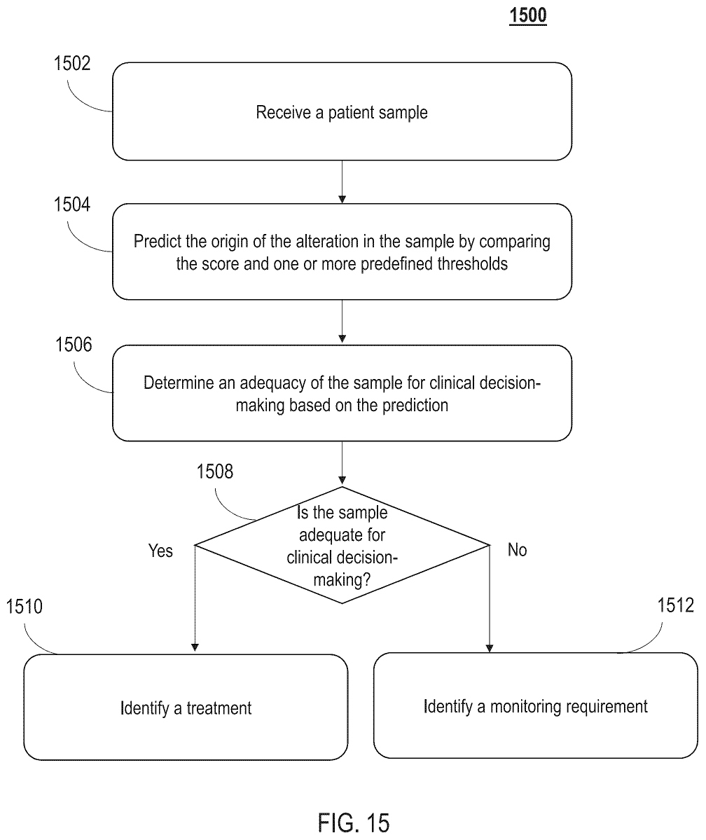

FIG. 15 provides a non-limiting example of a flowchart for a process 1500 for identifying a monitoring requirement for a patient based on the prediction according to embodiments of the present disclosure.

Process 1500 can be performed, for example, using one or more electronic devices implementing a software platform. In some examples, process 1500 is performed using a client-server system, and the blocks of process 1500 are divided up in any manner between the server and a client device. In other examples, the blocks of process 1500 are divided up between the server and multiple client devices. Thus, while portions of process 1500 are described herein as being performed by particular devices of a client-server system, it will be appreciated that process 1500 is not so limited. In other examples, process 1500 is performed using only a client device or only multiple client devices. In process 1500, some blocks are, optionally, combined, the order of some blocks is, optionally, changed, and some blocks are, optionally, omitted. In some examples, additional steps may be performed in combination with the process 1500. Accordingly, the operations as illustrated (and described in greater detail below) are exemplary by nature and, as such, should not be viewed as limiting.

At step 1502 in FIG. 15, the system can receive a patient sample. In some instances the patient sample can correspond to a liquid biopsy sample taken from a patient.

At step 1504 in FIG. 15, the system can predict the origin of the alteration in the sample by comparing the score and one or more predefined thresholds. In one or more examples, step 1504 can correspond to any of processes 200A and 200B described above.

At step 1506 in FIG. 15, the system can determine an adequacy of the sample for clinical decision-making based on the prediction. For example, if the prediction indicates that the plurality of reads corresponding to the alteration of interest or any alterations observed in the sample are CH-derived, then the system may determine that the sample is inadequate for clinical decision-making. That is, because the sequence read data corresponding to the alterations of interest do not include tumor-derived cells, the sample itself is unlikely to be useful to prescribe a treatment (e.g., cancer treatment) to the patient. Such a result may be used in clinical decision making, including the decision of whether genomic testing of tumor tissue, rather than cfDNA, is medically necessary.

At step 1508 in FIG. 15, if the system determines that the sample is adequate for clinical decision making, the system can proceed to step 1512, where the system can identify a treatment for the patient based on the prediction and the patient sample. For example, if the system determines that the alteration of interest is tumor derived, the system can identify a treatment for the patient based on the sequence read data from the patient sample.

In one or more examples, at step 1508 in FIG. 15, the system determines that the sample is not adequate for clinical decision making, the system can proceed to step 1512, where the system can identify a monitoring requirement for the patient. For example, the system can determine that further testing is needed based on the output score from the machine learning model and whether the sample is determined to be adequate for clinical decision-making. In one or more examples, the further testing can correspond to the monitoring requirement described above.

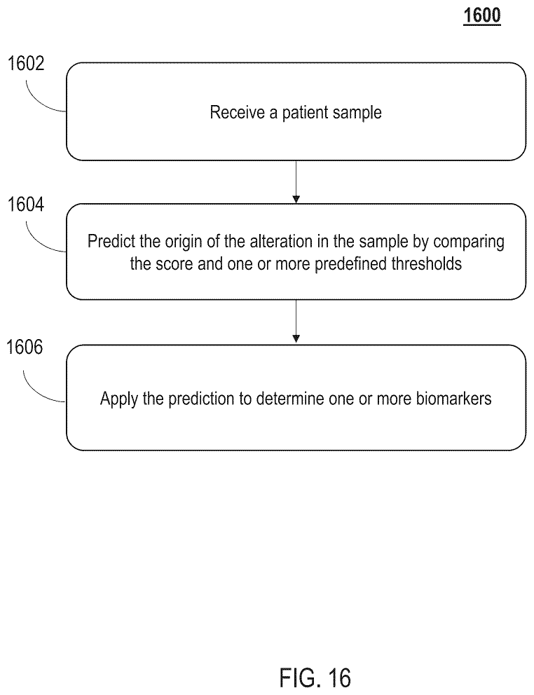

FIG. 16 provides a non-limiting example of a flowchart for a process 1600 for determining one or more biomarkers according to embodiments of the present disclosure.

Process 1600 can be performed, for example, using one or more electronic devices implementing a software platform. In some examples, process 1600 is performed using a client-server system, and the blocks of process 1600 are divided up in any manner between the server and a client device. In other examples, the blocks of process 1600 are divided up between the server and multiple client devices. Thus, while portions of process 1600 are described herein as being performed by particular devices of a client-server system, it will be appreciated that process 1600 is not so limited. In other examples, process 1600 is performed using only a client device or only multiple client devices. In process 1600, some blocks are, optionally, combined, the order of some blocks is, optionally, changed, and some blocks are, optionally, omitted. In some examples, additional steps may be performed in combination with the process 1600. Accordingly, the operations as illustrated (and described in greater detail below) are exemplary by nature and, as such, should not be viewed as limiting.

At step 1602 in FIG. 16, the system can receive a patient sample. In some instances the patient sample can correspond to a liquid biopsy sample taken from a patient.

At step 1604 in FIG. 16, the system can predict the origin of the alteration in the sample by comparing the score and one or more predefined thresholds. In one or more examples, step 1604 can correspond to any of processes 200A and 200B described above.

At step 1606 in FIG. 16, the system can apply the prediction to determine one or more biomarkers. In one or more examples, the system may apply the prediction to determine one of more biomarkers if the system predicts that the alteration of interest is tumor-derived. In one or more examples, the system may apply the prediction to determine one or more biomarkers if the system determines that the sample is adequate for clinical decision making as described in process 1500. Accordingly, based on the prediction that the alteration in the sample is tumor-derived, the system can use the CH versus tumor predictions in downstream analyses to determine biomarkers such as, but not limited to tumor fraction, bTMB, measures of microsatellite instability, homologous recombination metrics, personalized neoantigen prediction schemes, mutational signatures, patient-specific cancer monitoring tests, and/or ctDNA quantification for monitoring or molecular residual disease determination. In one or more examples, the system can identify a treatment based on the one or more biomarkers.

In some instances, the disclosed methods may be used to identify variants in the ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOTIL, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MSTIR, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2RIA, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSCIL1, WT1, XPO1, XRCC2, ZNF217, or ZNF703 gene locus, or any combination thereof.

In some instances, the disclosed methods may be used to identify variants in the ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1β, IL-6, IL-6R, JAK1, JAK2, JAK3, KIT, KRAS, MEK, MET, MSI-H, mTOR, PARP, PD-1, PDGFR, PDGFRα, PDGFRB, PD-L1, PI3Kδ, PIGF, PTCH, RAF, RANKL, RET, ROS1, SLAMF7, VEGF, VEGFA, or VEGFB gene locus, or any combination thereof.

### Methods of Use

In some instances, the disclosed methods may further comprise one or more of the steps of: (i) obtaining the sample from the subject (e.g., a subject suspected of having or determined to have cancer), (ii) extracting nucleic acid molecules (e.g., a mixture of tumor nucleic acid molecules and non-tumor nucleic acid molecules) from the sample, (iii) ligating one or more adapters to the nucleic acid molecules extracted from the sample (e.g., one or more amplification primers, flow cell adaptor sequences, substrate adapter sequences, or sample index sequences), (iv) amplifying the nucleic acid molecules (e.g., using a polymerase chain reaction (PCR) amplification technique, a non-PCR amplification technique, or an isothermal amplification technique), (v) capturing nucleic acid molecules from the amplified nucleic acid molecules (e.g., by hybridization to one or more bait molecules, where the bait molecules each comprise one or more nucleic acid molecules that each comprising a region that is complementary to a region of a captured nucleic acid molecule), (vi) sequencing the nucleic acid molecules extracted from the sample (or library proxies derived therefrom) using, e.g., a next-generation (massively parallel) sequencing technique, a whole genome sequencing (WGS) technique, a whole exome sequencing technique, a targeted sequencing technique, a direct sequencing technique, or a Sanger sequencing technique) using, e.g., a next-generation (massively parallel) sequencer, and (vii) generating, displaying, transmitting, and/or delivering a report (e.g., an electronic, web-based, or paper report) to the subject (or patient), a caregiver, a healthcare provider, a physician, an oncologist, an electronic medical record system, a hospital, a clinic, a third-party payer, an insurance company, or a government office. In some instances, the report comprises output from the methods described herein. In some instances, all or a portion of the report may be displayed in the graphical user interface of an online or web-based healthcare portal. In some instances, the report is transmitted via a computer network or peer-to-peer connection.

The disclosed methods may be used with any of a variety of samples. For example, in some instances, the sample may comprise a tissue biopsy sample, a liquid biopsy sample, or a normal control. In some instances, the sample may be a liquid biopsy sample and may comprise blood, plasma, cerebrospinal fluid, sputum, stool, urine, or saliva. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the nucleic acid molecules extracted from a sample may comprise a mixture of tumor nucleic acid molecules and non-tumor nucleic acid molecules. In some instances, the tumor nucleic acid molecules may be derived from a tumor portion of a heterogeneous tissue biopsy sample, and the non-tumor nucleic acid molecules may be derived from a normal portion of the heterogeneous tissue biopsy sample. In some instances, the sample may comprise a liquid biopsy sample, and the tumor nucleic acid molecules may be derived from a circulating tumor DNA (ctDNA) fraction of the liquid biopsy sample while the non-tumor nucleic acid molecules may be derived from a non-tumor, cell-free DNA (cfDNA) fraction of the liquid biopsy sample.

In some instances, the disclosed methods for predicting an origin of a plurality of reads corresponding to an alteration of interest in a patient sample may be used to diagnose (or as part of a diagnosis of) the presence of disease or other condition (e.g., cancer, genetic disorders (such as Down Syndrome and Fragile X), neurological disorders, or any other disease type where detection of variants, e.g., copy number alternations, are relevant to diagnosing, treating, or predicting said disease) in a subject (e.g., a patient). In some instances, the disclosed methods may be applicable to diagnosis of any of a variety of cancers as described elsewhere herein.

In some instances, the disclosed methods for predicting an origin of a plurality of reads corresponding to an alteration of interest in a patient sample may be used to select a subject (e.g., a patient) for a clinical trial based on the prediction score indicative of an origin of an alteration of interest determined for one or more gene loci. In some instances, patient selection for clinical trials based on, e.g., a prediction of an origin of an alteration of interest at one or more gene loci, may accelerate the development of targeted therapies and improve the healthcare outcomes for treatment decisions.

In some instances, the disclosed methods for predicting an origin of a plurality of reads corresponding to an alteration of interest in a subject sample may be used to select an appropriate therapy or treatment (e.g., an anti-cancer therapy or anti-cancer treatment) for a subject. In some instances, for example, the anti-cancer therapy or treatment may comprise use of a poly (ADP-ribose) polymerase inhibitor (PARPi), a platinum compound, chemotherapy, radiation therapy, a targeted therapy (e.g., immunotherapy), surgery, or any combination thereof.

In some instances, the targeted therapy (or anti-cancer target therapy) may comprise abemaciclib (Verzenio), abiraterone acetate (Zytiga), acalabrutinib (Calquence), ado-trastuzumab emtansine (Kadcyla), afatinib dimaleate (Gilotrif), aldesleukin (Proleukin), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), copanlisib hydrochloride (Aliqopa), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), iobenguane I131 (Azedra), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), panobinostat (Farydak), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sipuleucel-T (Provenge), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.

In some instances, the disclosed methods for predicting an origin of an alteration of interest of a plurality of reads in a subject sample may be used in selecting treatment and/or treating a disease (e.g., a cancer) in a subject (e.g., a patient). For example, in response to predicting an origin of an alteration of interest of a plurality of reads using any of the methods disclosed herein, an effective amount of an anti-cancer therapy or anti-cancer treatment may be administered to the subject.

In some instances, the disclosed methods for predicting an origin of an alteration of interest of a plurality of reads in a subject sample may be used for monitoring disease progression or recurrence (e.g., cancer or tumor progression or recurrence) in a subject. For example, in some instances, the methods may be used to predict an origin of an alteration of interest of a plurality of reads in a first sample obtained from the subject at a first time point, and used to predicting an origin of the alteration of interest of a plurality of reads in a second sample obtained from the subject at a second time point, where comparison of the first prediction of the origin of the alteration of interest of the first plurality of reads and the second prediction of the origin of the alteration of interest of the first plurality of reads allows one to monitor disease progression or recurrence. In some instances, the first time point is chosen before the subject has been administered a therapy or treatment, and the second time point is chosen after the subject has been administered the therapy or treatment.

In some instances, the disclosed methods may be used for adjusting a therapy or treatment (e.g., an anti-cancer treatment or anti-cancer therapy) for a subject, e.g., by adjusting a treatment dose and/or selecting a different treatment in response to a change in the prediction of the origin of the alteration of interest of a plurality of reads in a subject's sample.

In some instances, the value of prediction score determined using the disclosed methods may be used as a prognostic or diagnostic indicator associated with the sample. For example, in some instances, the prognostic or diagnostic indicator may comprise an indicator of the presence of a disease (e.g., cancer) in the sample, an indicator of the probability that a disease (e.g., cancer) is present in the sample, an indicator of the probability that the subject from which the sample was derived will develop a disease (e.g., cancer) (i.e., a risk factor), or an indicator of the likelihood that the subject from which the sample was derived will respond to a particular therapy or treatment.

In some instances, the disclosed methods for predicting an origin of a plurality of reads corresponding to an alteration of interest in a subject sample may be implemented as part of a genomic profiling process that comprises identification of the presence of variant sequences at one or more gene loci in a sample derived from a subject as part of detecting, monitoring, predicting a risk factor, or selecting a treatment for a particular disease, e.g., cancer. In some instances, the variant panel selected for genomic profiling may comprise the detection of variant sequences at a selected set of gene loci. In some instances, the variant panel selected for genomic profiling may comprise detection of variant sequences at a number of gene loci through comprehensive genomic profiling (CGP), which is a next-generation sequencing (NGS) approach used to assess hundreds of genes (including relevant cancer biomarkers) in a single assay. Inclusion of the disclosed methods for predicting an origin of a plurality of reads corresponding to an alteration of interest in a subject sample as part of a genomic profiling process (or inclusion of the output from the disclosed methods for predicting an origin of a plurality of reads corresponding to an alteration of interest in a subject sample as part of the genomic profile of the subject) can improve the validity of, e.g., disease detection calls and treatment decisions, made on the basis of the genomic profile by, for example, independently confirming the presence of tumor-derived alterations in a given patient sample.

In some instances, a genomic profile may comprise information on the presence of genes (or variant sequences thereof), copy number variations, epigenetic traits, proteins (or modifications thereof), and/or other biomarkers in an individual's genome and/or proteome, as well as information on the individual's corresponding phenotypic traits and the interaction between genetic or genomic traits, phenotypic traits, and environmental factors.

In some instances, a genomic profile for the subject may comprise results from a comprehensive genomic profiling (CGP) test, a nucleic acid sequencing-based test, a gene expression profiling test, a cancer hotspot panel test, a DNA methylation test, a DNA fragmentation test, an RNA fragmentation test, or any combination thereof.

In some instances, the method can further include administering or applying a treatment or therapy (e.g., an anti-cancer agent, anti-cancer treatment, or anti-cancer therapy) to the subject based on the generated genomic profile. An anti-cancer agent or anti-cancer treatment may refer to a compound that is effective in the treatment of cancer cells. Examples of anti-cancer agents or anti-cancer therapies include, but not limited to, alkylating agents, antimetabolites, natural products, hormones, chemotherapy, radiation therapy, immunotherapy, surgery, or a therapy configured to target a defect in a specific cell signaling pathway, e.g., a defect in a DNA mismatch repair (MMR) pathway.

### Samples

The disclosed methods and systems may be used with any of a variety of samples (also referred to herein as specimens) comprising nucleic acids (e.g., DNA or RNA) that are collected from a subject (e.g., a patient). Examples of a sample include, but are not limited to, a tumor sample, a tissue sample, a biopsy sample (e.g., a tissue biopsy, a liquid biopsy, or both), a blood sample (e.g., a peripheral whole blood sample), a blood plasma sample, a blood serum sample, a lymph sample, a saliva sample, a sputum sample, a urine sample, a gynecological fluid sample, a circulating tumor cell (CTC) sample, a cerebral spinal fluid (CSF) sample, a pericardial fluid sample, a pleural fluid sample, an ascites (peritoneal fluid) sample, a feces (or stool) sample, or other body fluid, secretion, and/or excretion sample (or cell sample derived therefrom). In certain instances, the sample may be frozen sample or a formalin-fixed paraffin-embedded (FFPE) sample.

In some instances, the sample may be collected by tissue resection (e.g., surgical resection), needle biopsy, bone marrow biopsy, bone marrow aspiration, skin biopsy, endoscopic biopsy, fine needle aspiration, oral swab, nasal swab, vaginal swab or a cytology smear, scrapings, washings or lavages (such as a ductal lavage or a bronchoalveolar lavage), etc.

In some instances, the sample is a liquid biopsy sample, and may comprise, e.g., whole blood, blood plasma, blood serum, urine, stool, sputum, saliva, or cerebrospinal fluid. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the sample may comprise one or more premalignant or malignant cells. Premalignant, as used herein, refers to a cell or tissue that is not yet malignant but is poised to become malignant. In certain instances, the sample may be acquired from a solid tumor, a soft tissue tumor, or a metastatic lesion. In certain instances, the sample may be acquired from a hematologic malignancy or pre-malignancy. In other instances, the sample may comprise a tissue or cells from a surgical margin. In certain instances, the sample may comprise tumor-infiltrating lymphocytes. In some instances, the sample may comprise one or more non-malignant cells. In some instances, the sample may be, or is part of, a primary tumor or a metastasis (e.g., a metastasis biopsy sample). In some instances, the sample may be obtained from a site (e.g., a tumor site) with the highest percentage of tumor (e.g., tumor cells) as compared to adjacent sites (e.g., sites adjacent to the tumor). In some instances, the sample may be obtained from a site (e.g., a tumor site) with the largest tumor focus (e.g., the largest number of tumor cells as visualized under a microscope) as compared to adjacent sites (e.g., sites adjacent to the tumor).

In some instances, the disclosed methods may further comprise analyzing a primary control (e.g., a normal tissue sample). In some instances, the disclosed methods may further comprise determining if a primary control is available and, if so, isolating a control nucleic acid (e.g., DNA) from said primary control. In some instances, the sample may comprise any normal control (e.g., a normal adjacent tissue (NAT)) if no primary control is available. In some instances, the sample may be or may comprise histologically normal tissue. In some instances, the method includes evaluating a sample, e.g., a histologically normal sample (e.g., from a surgical tissue margin) using the methods described herein. In some instances, the disclosed methods may further comprise acquiring a sub-sample enriched for non-tumor cells, e.g., by macro-dissecting non-tumor tissue from said NAT in a sample not accompanied by a primary control. In some instances, the disclosed methods may further comprise determining that no primary control and no NAT is available, and marking said sample for analysis without a matched control.

In some instances, samples obtained from histologically normal tissues (e.g., otherwise histologically normal surgical tissue margins) may still comprise a genetic alteration such as a variant sequence as described herein. The methods may thus further comprise re-classifying a sample based on the presence of the detected genetic alteration. In some instances, multiple samples (e.g., from different subjects) are processed simultaneously.

The disclosed methods and systems may be applied to the analysis of nucleic acids extracted from any of variety of tissue samples (or disease states thereof), e.g., solid tissue samples, soft tissue samples, metastatic lesions, or liquid biopsy samples. Examples of tissues include, but are not limited to, connective tissue, muscle tissue, nervous tissue, epithelial tissue, and blood. Tissue samples may be collected from any of the organs within an animal or human body. Examples of human organs include, but are not limited to, the brain, heart, lungs, liver, kidneys, pancreas, spleen, thyroid, mammary glands, uterus, prostate, large intestine, small intestine, bladder, bone, skin, etc.

In some instances, the nucleic acids extracted from the sample may comprise deoxyribonucleic acid (DNA) molecules. Examples of DNA that may be suitable for analysis by the disclosed methods include, but are not limited to, genomic DNA or fragments thereof, mitochondrial DNA or fragments thereof, cell-free DNA (cfDNA), and circulating tumor DNA (ctDNA). Cell-free DNA (cfDNA) is comprised of fragments of DNA that are released from normal and/or cancerous cells during apoptosis and necrosis, and circulate in the blood stream and/or accumulate in other bodily fluids. Circulating tumor DNA (ctDNA) is comprised of fragments of DNA that are released from cancerous cells and tumors that circulate in the blood stream and/or accumulate in other bodily fluids.

In some instances, DNA is extracted from nucleated cells from the sample. In some instances, a sample may have a low nucleated cellularity, e.g., when the sample is comprised mainly of erythrocytes, lesional cells that contain excessive cytoplasm, or tissue with fibrosis. In some instances, a sample with low nucleated cellularity may require more, e.g., greater, tissue volume for DNA extraction.

In some instances, the nucleic acids extracted from the sample may comprise ribonucleic acid (RNA) molecules. Examples of RNA that may be suitable for analysis by the disclosed methods include, but are not limited to, total cellular RNA, total cellular RNA after depletion of certain abundant RNA sequences (e.g., ribosomal RNAs), cell-free RNA (cfRNA), messenger RNA (mRNA) or fragments thereof, the poly(A)-tailed mRNA fraction of the total RNA, ribosomal RNA (rRNA) or fragments thereof, transfer RNA (tRNA) or fragments thereof, and mitochondrial RNA or fragments thereof. In some instances, RNA may be extracted from the sample and converted to complementary DNA (cDNA) using, e.g., a reverse transcription reaction. In some instances, the cDNA is produced by random-primed cDNA synthesis methods. In other instances, the cDNA synthesis is initiated at the poly(A) tail of mature mRNAs by priming with oligo (dT)-containing oligonucleotides. Methods for depletion, poly(A) enrichment, and cDNA synthesis are well known to those of skill in the art.

In some instances, the sample may comprise a tumor content (e.g., comprising tumor cells or tumor cell nuclei), or a non-tumor content (e.g., immune cells, fibroblasts, and other non-tumor cells). In some instances, the tumor content of the sample may constitute a sample metric. In some instances, the sample may comprise a tumor content of at least 5-50%, 10-40%, 15-25%, or 20-30% tumor cell nuclei. In some instances, the sample may comprise a tumor content of at least 5%, at least 10%, at least 20%, at least 30%, at least 40%, or at least 50% tumor cell nuclei. In some instances, the percent tumor cell nuclei (e.g., sample fraction) is determined (e.g., calculated) by dividing the number of tumor cells in the sample by the total number of all cells within the sample that have nuclei. In some instances, for example when the sample is a liver sample comprising hepatocytes, a different tumor content calculation may be required due to the presence of hepatocytes having nuclei with twice, or more than twice, the DNA content of other, e.g., non-hepatocyte, somatic cell nuclei. In some instances, the sensitivity of detection of a genetic alteration, e.g., a variant sequence, or a determination of, e.g., microsatellite instability, may depend on the tumor content of the sample. For example, a sample having a lower tumor content can result in lower sensitivity of detection for a given size sample.

In some instances, as noted above, the sample comprises nucleic acid (e.g., DNA, RNA (or a cDNA derived from the RNA), or both), e.g., from a tumor or from normal tissue. In certain instances, the sample may further comprise a non-nucleic acid component, e.g., cells, protein, carbohydrate, or lipid, e.g., from the tumor or normal tissue.

### Subjects

In some instances, the sample is obtained (e.g., collected) from a subject (e.g., patient) with a condition or disease (e.g., a hyperproliferative disease or a non-cancer indication) or suspected of having the condition or disease. In some instances, the hyperproliferative disease is a cancer. In some instances, the cancer is a solid tumor or a metastatic form thereof. In some instances, the cancer is a hematological cancer, e.g., a leukemia or lymphoma.

In some instances, the subject has a cancer or is at risk of having a cancer. For example, in some instances, the subject has a genetic predisposition to a cancer (e.g., having a genetic mutation that increases his or her baseline risk for developing a cancer). In some instances, the subject has been exposed to an environmental perturbation (e.g., radiation or a chemical) that increases his or her risk for developing a cancer. In some instances, the subject is in need of being monitored for development of a cancer. In some instances, the subject is in need of being monitored for cancer progression or regression, e.g., after being treated with an anti-cancer therapy (or anti-cancer treatment). In some instances, the subject is in need of being monitored for relapse of cancer. In some instances, the subject is in need of being monitored for minimum residual disease (MRD). In some instances, the subject has been, or is being treated, for cancer. In some instances, the subject has not been treated with an anti-cancer therapy (or anti-cancer treatment).

In some instances, the subject (e.g., a patient) is being treated, or has been previously treated, with one or more targeted therapies. In some instances, e.g., for a patient who has been previously treated with a targeted therapy, a post-targeted therapy sample (e.g., specimen) is obtained (e.g., collected). In some instances, the post-targeted therapy sample is a sample obtained after the completion of the targeted therapy.

In some instances, the patient has not been previously treated with a targeted therapy. In some instances, e.g., for a patient who has not been previously treated with a targeted therapy, the sample comprises a resection, e.g., an original resection, or a resection following recurrence (e.g., following a disease recurrence post-therapy).

### Cancers

In some instances, the sample is acquired from a subject having a cancer. Exemplary cancers include, but are not limited to, B cell cancer (e.g., multiple myeloma), melanomas, breast cancer, lung cancer (such as non-small cell lung carcinoma or NSCLC), bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, adenocarcinomas, inflammatory myofibroblastic tumors, gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancers, essential thrombocythemia, agnogenic myeloid metaplasia, hypereosinophilic syndrome, systemic mastocytosis, familiar hypereosinophilia, chronic eosinophilic leukemia, neuroendocrine cancers, carcinoid tumors, and the like.

In some instances, the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR and MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), a gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

In some instances, the cancer is a hematologic malignancy (or premaligancy). As used herein, a hematologic malignancy refers to a tumor of the hematopoietic or lymphoid tissues, e.g., a tumor that affects blood, bone marrow, or lymph nodes. Exemplary hematologic malignancies include, but are not limited to, leukemia (e.g., acute lymphoblastic leukemia (ALL), acute myeloid leukemia (AML), chronic lymphocytic leukemia (CLL), chronic myelogenous leukemia (CML), hairy cell leukemia, acute monocytic leukemia (AMoL), chronic myelomonocytic leukemia (CMML), juvenile myelomonocytic leukemia (JMML), or large granular lymphocytic leukemia), lymphoma (e.g., AIDS-related lymphoma, cutaneous T-cell lymphoma, Hodgkin lymphoma (e.g., classical Hodgkin lymphoma or nodular lymphocyte-predominant Hodgkin lymphoma), mycosis fungoides, non-Hodgkin lymphoma (e.g., B-cell non-Hodgkin lymphoma (e.g., Burkitt lymphoma, small lymphocytic lymphoma (CLL/SLL), diffuse large B-cell lymphoma, follicular lymphoma, immunoblastic large cell lymphoma, precursor B-lymphoblastic lymphoma, or mantle cell lymphoma) or T-cell non-Hodgkin lymphoma (mycosis fungoides, anaplastic large cell lymphoma, or precursor T-lymphoblastic lymphoma)), primary central nervous system lymphoma, Sézary syndrome, Waldenström macroglobulinemia), chronic myeloproliferative neoplasm, Langerhans cell histiocytosis, multiple myeloma/plasma cell neoplasm, myelodysplastic syndrome, or myelodysplastic/myeloproliferative neoplasm.

### Nucleic Acid Extraction and Processing

DNA or RNA may be extracted from tissue samples, biopsy samples, blood samples, or other bodily fluid samples using any of a variety of techniques known to those of skill in the art (see, e.g., Example 1 of International Patent Application Publication No. WO 2012/092426; Tan, et al. (2009), “DNA, RNA, and Protein Extraction: The Past and The Present”, J. Biomed. Biotech. 2009:574398; the technical literature for the Maxwell® 16 LEV Blood DNA Kit (Promega Corporation, Madison, WI); and the Maxwell 16 Buccal Swab LEV DNA Purification Kit Technical Manual (Promega Literature #TM333, Jan. 1, 2011, Promega Corporation, Madison, WI)). Protocols for RNA isolation are disclosed in, e.g., the Maxwell® 16 Total RNA Purification Kit Technical Bulletin (Promega Literature #TB351, August 2009, Promega Corporation, Madison, WI).

A typical DNA extraction procedure, for example, comprises (i) collection of the fluid sample, cell sample, or tissue sample from which DNA is to be extracted, (ii) disruption of cell membranes (i.e., cell lysis), if necessary, to release DNA and other cytoplasmic components, (iii) treatment of the fluid sample or lysed sample with a concentrated salt solution to precipitate proteins, lipids, and RNA, followed by centrifugation to separate out the precipitated proteins, lipids, and RNA, and (iv) purification of DNA from the supernatant to remove detergents, proteins, salts, or other reagents used during the cell membrane lysis step.

Disruption of cell membranes may be performed using a variety of mechanical shear (e.g., by passing through a French press or fine needle) or ultrasonic disruption techniques. The cell lysis step often comprises the use of detergents and surfactants to solubilize lipids the cellular and nuclear membranes. In some instances, the lysis step may further comprise use of proteases to break down protein, and/or the use of an RNase for digestion of RNA in the sample.

Examples of suitable techniques for DNA purification include, but are not limited to, (i) precipitation in ice-cold ethanol or isopropanol, followed by centrifugation (precipitation of DNA may be enhanced by increasing ionic strength, e.g., by addition of sodium acetate), (ii) phenol-chloroform extraction, followed by centrifugation to separate the aqueous phase containing the nucleic acid from the organic phase containing denatured protein, and (iii) solid phase chromatography where the nucleic acids adsorb to the solid phase (e.g., silica or other) depending on the pH and salt concentration of the buffer.

In some instances, cellular and histone proteins bound to the DNA may be removed either by adding a protease or by having precipitated the proteins with sodium or ammonium acetate, or through extraction with a phenol-chloroform mixture prior to a DNA precipitation step.

In some instances, DNA may be extracted using any of a variety of suitable commercial DNA extraction and purification kits. Examples include, but are not limited to, the QIAamp (for isolation of genomic DNA from human samples) and DNAeasy (for isolation of genomic DNA from animal or plant samples) kits from Qiagen (Germantown, MD) or the Maxwell® and ReliaPrep™ series of kits from Promega (Madison, WI).

As noted above, in some instances the sample may comprise a formalin-fixed (also known as formaldehyde-fixed, or paraformaldehyde-fixed), paraffin-embedded (FFPE) tissue preparation. For example, the FFPE sample may be a tissue sample embedded in a matrix, e.g., an FFPE block. Methods to isolate nucleic acids (e.g., DNA) from formaldehyde- or paraformaldehyde-fixed, paraffin-embedded (FFPE) tissues are disclosed in, e.g., Cronin, et al., (2004) Am J Pathol. 164 (1): 35-42; Masuda, et al., (1999) Nucleic Acids Res. 27 (22): 4436-4443; Specht, et al., (2001) Am J Pathol. 158 (2): 419-429; the Ambion RecoverAll™ Total Nucleic Acid Isolation Protocol (Ambion, Cat. No. AM1975, September 2008); the Maxwell® 16 FFPE Plus LEV DNA Purification Kit Technical Manual (Promega Literature #TM349, February 2011); the E.Z.N.A.® FFPE DNA Kit Handbook (OMEGA bio-tek, Norcross, GA, product numbers D3399-00, D3399-01, and D3399-02, June 2009); and the QIAamp® DNA FFPE Tissue Handbook (Qiagen, Cat. No. 37625, October 2007). For example, the RecoverAll™ Total Nucleic Acid Isolation Kit uses xylene at elevated temperatures to solubilize paraffin-embedded samples and a glass-fiber filter to capture nucleic acids. The Maxwell® 16 FFPE Plus LEV DNA Purification Kit is used with the Maxwell® 16 Instrument for purification of genomic DNA from 1 to 10 μm sections of FFPE tissue. DNA is purified using silica-clad paramagnetic particles (PMPs), and eluted in low elution volume. The E.Z.N.A.® FFPE DNA Kit uses a spin column and buffer system for isolation of genomic DNA. QIAamp® DNA FFPE Tissue Kit uses QIAamp® DNA Micro technology for purification of genomic and mitochondrial DNA.

In some instances, the disclosed methods may further comprise determining or acquiring a yield value for the nucleic acid extracted from the sample and comparing the determined value to a reference value. For example, if the determined or acquired value is less than the reference value, the nucleic acids may be amplified prior to proceeding with library construction. In some instances, the disclosed methods may further comprise determining or acquiring a value for the size (or average size) of nucleic acid fragments in the sample, and comparing the determined or acquired value to a reference value, e.g., a size (or average size) of at least 100, 200, 300, 400, 500, 600, 700, 800, 900, or 1000 base pairs (bps). In some instances, one or more parameters described herein may be adjusted or selected in response to this determination.

After isolation, the nucleic acids are typically dissolved in a slightly alkaline buffer, e.g., Tris-EDTA (TE) buffer, or in ultra-pure water. In some instances, the isolated nucleic acids (e.g., genomic DNA) may be fragmented or sheared by using any of a variety of techniques known to those of skill in the art. For example, genomic DNA can be fragmented by physical shearing methods, enzymatic cleavage methods, chemical cleavage methods, and other methods known to those of skill in the art. Methods for DNA shearing are described in Example 4 in International Patent Application Publication No. WO 2012/092426. In some instances, alternatives to DNA shearing methods can be used to avoid a ligation step during library preparation.

### Library Preparation

In some instances, the nucleic acids isolated from the sample may be used to construct a library (e.g., a nucleic acid library as described herein). In some instances, the nucleic acids are fragmented using any of the methods described above, optionally subjected to repair of chain end damage, and optionally ligated to synthetic adapters, primers, and/or barcodes (e.g., amplification primers, sequencing adapters, flow cell adapters, substrate adapters, sample barcodes or indexes, and/or unique molecular identifier sequences), size-selected (e.g., by preparative gel electrophoresis), and/or amplified (e.g., using PCR, a non-PCR amplification technique, or an isothermal amplification technique). In some instances, the fragmented and adapter-ligated group of nucleic acids is used without explicit size selection or amplification prior to hybridization-based selection of target sequences. In some instances, the nucleic acid is amplified by any of a variety of specific or nonspecific nucleic acid amplification methods known to those of skill in the art. In some instances, the nucleic acids are amplified, e.g., by a whole-genome amplification method such as random-primed strand-displacement amplification. Examples of nucleic acid library preparation techniques for next-generation sequencing are described in, e.g., van Dijk, et al. (2014), Exp. Cell Research 322:12-20, and Illumina's genomic DNA sample preparation kit.

In some instances, the resulting nucleic acid library may contain all or substantially all of the complexity of the genome. The term “substantially all” in this context refers to the possibility that there can in practice be some unwanted loss of genome complexity during the initial steps of the procedure. The methods described herein also are useful in cases where the nucleic acid library comprises a portion of the genome, e.g., where the complexity of the genome is reduced by design. In some instances, any selected portion of the genome can be used with a method described herein. For example, in certain embodiments, the entire exome or a subset thereof is isolated. In some instances, the library may include at least 95%, 90%, 80%, 70%, 60%, 50%, 40%, 30%, 20%, 10%, or 5% of the genomic DNA. In some instances, the library may consist of cDNA copies of genomic DNA that includes copies of at least 95%, 90%, 80%, 70%, 60%, 50%, 40%, 30%, 20%, 10%, or 5% of the genomic DNA. In certain instances, the amount of nucleic acid used to generate the nucleic acid library may be less than 5 micrograms, less than 1 microgram, less than 500 ng, less than 200 ng, less than 100 ng, less than 50 ng, less than 10 ng, less than 5 ng, or less than 1 ng.

In some instances, a library (e.g., a nucleic acid library) includes a collection of nucleic acid molecules. As described herein, the nucleic acid molecules of the library can include a target nucleic acid molecule (e.g., a tumor nucleic acid molecule, a reference nucleic acid molecule and/or a control nucleic acid molecule; also referred to herein as a first, second and/or third nucleic acid molecule, respectively). The nucleic acid molecules of the library can be from a single subject or individual. In some instances, a library can comprise nucleic acid molecules derived from more than one subject (e.g., 2, 3, 4, 5, 6, 7, 8, 9, 10, 20, 30 or more subjects). For example, two or more libraries from different subjects can be combined to form a library having nucleic acid molecules from more than one subject (where the nucleic acid molecules derived from each subject are optionally ligated to a unique sample barcode corresponding to a specific subject). In some instances, the subject is a human having, or at risk of having, a cancer or tumor.

In some instances, the library (or a portion thereof) may comprise one or more subgenomic intervals. In some instances, a subgenomic interval can be a single nucleotide position, e.g., a nucleotide position for which a variant at the position is associated (positively or negatively) with a tumor phenotype. In some instances, a subgenomic interval comprises more than one nucleotide position. Such instances include sequences of at least 2, 5, 10, 50, 100, 150, 250, or more than 250 nucleotide positions in length. Subgenomic intervals can comprise, e.g., one or more entire genes (or portions thereof), one or more exons or coding sequences (or portions thereof), one or more introns (or portion thereof), one or more microsatellite region (or portions thereof), or any combination thereof. A subgenomic interval can comprise all or a part of a fragment of a naturally occurring nucleic acid molecule, e.g., a genomic DNA molecule. For example, a subgenomic interval can correspond to a fragment of genomic DNA which is subjected to a sequencing reaction. In some instances, a subgenomic interval is a continuous sequence from a genomic source. In some instances, a subgenomic interval includes sequences that are not contiguous in the genome, e.g., subgenomic intervals in cDNA can include exon-exon junctions formed as a result of splicing. In some instances, the subgenomic interval comprises a tumor nucleic acid molecule. In some instances, the subgenomic interval comprises a non-tumor nucleic acid molecule.

### Targeting Gene Loci for Analysis

The methods described herein can be used in combination with, or as part of, a method for evaluating a plurality or set of subject intervals (e.g., target sequences), e.g., from a set of genomic loci (e.g., gene loci or fragments thereof), as described herein.

In some instances, the set of genomic loci evaluated by the disclosed methods comprises a plurality of, e.g., genes, which in mutant form, are associated with an effect on cell division, growth or survival, or are associated with a cancer, e.g., a cancer described herein.

In some instances, the set of gene loci evaluated by the disclosed methods comprises at least 1, at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, or more than 100 gene loci.

In some instances, the selected gene loci (also referred to herein as target gene loci or target sequences), or fragments thereof, may include subject intervals comprising non-coding sequences, coding sequences, intragenic regions, or intergenic regions of the subject genome. For example, the subject intervals can include a non-coding sequence or fragment thereof (e.g., a promoter sequence, enhancer sequence, 5′ untranslated region (5′ UTR), 3′ untranslated region (3′ UTR), or a fragment thereof), a coding sequence of fragment thereof, an exon sequence or fragment thereof, an intron sequence or a fragment thereof.

### Target Capture Reagents

The methods described herein may comprise contacting a nucleic acid library with a plurality of target capture reagents in order to select and capture a plurality of specific target sequences (e.g., gene sequences or fragments thereof) for analysis. In some instances, a target capture reagent (i.e., a molecule which can bind to and thereby allow capture of a target molecule) is used to select the subject intervals to be analyzed. For example, a target capture reagent can be a bait molecule, e.g., a nucleic acid molecule (e.g., a DNA molecule or RNA molecule) which can hybridize to (i.e., is complementary to) a target molecule, and thereby allows capture of the target nucleic acid. In some instances, the target capture reagent, e.g., a bait molecule (or bait sequence), is a capture oligonucleotide (or capture probe). In some instances, the target nucleic acid is a genomic DNA molecule, an RNA molecule, a cDNA molecule derived from an RNA molecule, a microsatellite DNA sequence, and the like. In some instances, the target capture reagent is suitable for solution-phase hybridization to the target. In some instances, the target capture reagent is suitable for solid-phase hybridization to the target. In some instances, the target capture reagent is suitable for both solution-phase and solid-phase hybridization to the target. The design and construction of target capture reagents is described in more detail in, e.g., International Patent Application Publication No. WO 2020/236941, the entire content of which is incorporated herein by reference.

The methods described herein provide for optimized sequencing of a large number of genomic loci (e.g., genes or gene products (e.g., mRNA), microsatellite loci, etc.) from samples (e.g., cancerous tissue specimens, liquid biopsy samples, and the like) from one or more subjects by the appropriate selection of target capture reagents to select the target nucleic acid molecules to be sequenced. In some instances, a target capture reagent may hybridize to a specific target locus, e.g., a specific target gene locus or fragment thereof. In some instances, a target capture reagent may hybridize to a specific group of target loci, e.g., a specific group of gene loci or fragments thereof. In some instances, a plurality of target capture reagents comprising a mix of target-specific and/or group-specific target capture reagents may be used.

In some instances, the number of target capture reagents (e.g., bait molecules) in the plurality of target capture reagents (e.g., a bait set) contacted with a nucleic acid library to capture a plurality of target sequences for nucleic acid sequencing is greater than 10, greater than 50, greater than 100, greater than 200, greater than 300, greater than 400, greater than 500, greater than 600, greater than 700, greater than 800, greater than 900, greater than 1,000, greater than 1,250, greater than 1,500, greater than 1,750, greater than 2,000, greater than 3,000, greater than 4,000, greater than 5,000, greater than 10,000, greater than 25,000, or greater than 50,000.

In some instances, the overall length of the target capture reagent sequence can be between about 70 nucleotides and 1000 nucleotides. In one instance, the target capture reagent length is between about 100 and 300 nucleotides, 110 and 200 nucleotides, or 120 and 170 nucleotides, in length. In addition to those mentioned above, intermediate oligonucleotide lengths of about 70, 80, 90, 100, 110, 120, 130, 140, 150, 160, 170, 180, 190, 200, 210, 220, 230, 240, 250, 300, 400, 500, 600, 700, 800, and 900 nucleotides in length can be used in the methods described herein. In some embodiments, oligonucleotides of about 70, 80, 90, 100, 110, 120, 130, 140, 150, 160, 170, 180, 190, 200, 210, 220, or 230 bases can be used.

In some instances, each target capture reagent sequence can include: (i) a target-specific capture sequence (e.g., a gene locus or microsatellite locus-specific complementary sequence), (ii) an adapter, primer, barcode, and/or unique molecular identifier sequence, and (iii) universal tails on one or both ends. As used herein, the term “target capture reagent” can refer to the target-specific target capture sequence or to the entire target capture reagent oligonucleotide including the target-specific target capture sequence.

In some instances, the target-specific capture sequences in the target capture reagents are between about 40 nucleotides and 1000 nucleotides in length. In some instances, the target-specific capture sequence is between about 70 nucleotides and 300 nucleotides in length. In some instances, the target-specific sequence is between about 100 nucleotides and 200 nucleotides in length. In yet other instances, the target-specific sequence is between about 120 nucleotides and 170 nucleotides in length, typically 120 nucleotides in length. Intermediate lengths in addition to those mentioned above also can be used in the methods described herein, such as target-specific sequences of about 40, 50, 60, 70, 80, 90, 100, 110, 120, 130, 140, 150, 160, 170, 180, 190, 200, 210, 220, 230, 240, 250, 300, 400, 500, 600, 700, 800, and 900 nucleotides in length, as well as target-specific sequences of lengths between the above-mentioned lengths.

In some instances, the target capture reagent may be designed to select a subject interval containing one or more rearrangements, e.g., an intron containing a genomic rearrangement. In such instances, the target capture reagent is designed such that repetitive sequences are masked to increase the selection efficiency. In those instances where the rearrangement has a known juncture sequence, complementary target capture reagents can be designed to recognize the juncture sequence to increase the selection efficiency.

In some instances, the disclosed methods may comprise the use of target capture reagents designed to capture two or more different target categories, each category having a different target capture reagent design strategy. In some instances, the hybridization-based capture methods and target capture reagent compositions disclosed herein may provide for the capture and homogeneous coverage of a set of target sequences, while minimizing coverage of genomic sequences outside of the targeted set of sequences. In some instances, the target sequences may include the entire exome of genomic DNA or a selected subset thereof. In some instances, the target sequences may include, e.g., a large chromosomal region (e.g., a whole chromosome arm). The methods and compositions disclosed herein provide different target capture reagents for achieving different sequencing depths and patterns of coverage for complex sets of target nucleic acid sequences.

Typically, DNA molecules are used as target capture reagent sequences, although RNA molecules can also be used. In some instances, a DNA molecule target capture reagent can be single stranded DNA (ssDNA) or double-stranded DNA (dsDNA). In some instances, an RNA-DNA duplex is more stable than a DNA-DNA duplex and therefore provides for potentially better capture of nucleic acids.

In some instances, the disclosed methods comprise providing a selected set of nucleic acid molecules (e.g., a library catch) captured from one or more nucleic acid libraries. For example, the method may comprise: providing one or a plurality of nucleic acid libraries, each comprising a plurality of nucleic acid molecules (e.g., a plurality of target nucleic acid molecules and/or reference nucleic acid molecules) extracted from one or more samples from one or more subjects; contacting the one or a plurality of libraries (e.g., in a solution-based hybridization reaction) with one, two, three, four, five, or more than five pluralities of target capture reagents (e.g., oligonucleotide target capture reagents) to form a hybridization mixture comprising a plurality of target capture reagent/nucleic acid molecule hybrids; separating the plurality of target capture reagent/nucleic acid molecule hybrids from said hybridization mixture, e.g., by contacting said hybridization mixture with a binding entity that allows for separation of said plurality of target capture reagent/nucleic acid molecule hybrids from the hybridization mixture, thereby providing a library catch (e.g., a selected or enriched subgroup of nucleic acid molecules from the one or a plurality of libraries).

In some instances, the disclosed methods may further comprise amplifying the library catch (e.g., by performing PCR). In other instances, the library catch is not amplified.

In some instances, the target capture reagents can be part of a kit which can optionally comprise instructions, standards, buffers or enzymes or other reagents.

### Hybridization Conditions

As noted above, the methods disclosed herein may include the step of contacting the library (e.g., the nucleic acid library) with a plurality of target capture reagents to provide a selected library target nucleic acid sequences (i.e., the library catch). The contacting step can be effected in, e.g., solution-based hybridization. In some instances, the method includes repeating the hybridization step for one or more additional rounds of solution-based hybridization. In some instances, the method further includes subjecting the library catch to one or more additional rounds of solution-based hybridization with the same or a different collection of target capture reagents.

In some instances, the contacting step is effected using a solid support, e.g., an array. Suitable solid supports for hybridization are described in, e.g., Albert, T. J. et al. (2007) Nat. Methods 4 (11): 903-5; Hodges, E. et al. (2007) Nat. Genet. 39 (12): 1522-7; and Okou, D. T. et al. (2007) Nat. Methods 4 (11): 907-9, the contents of which are incorporated herein by reference in their entireties.

Hybridization methods that can be adapted for use in the methods herein are described in the art, e.g., as described in International Patent Application Publication No. WO 2012/092426. Methods for hybridizing target capture reagents to a plurality of target nucleic acids are described in more detail in, e.g., International Patent Application Publication No. WO 2020/236941, the entire content of which is incorporated herein by reference.

### Sequencing Methods

The methods and systems disclosed herein can be used in combination with, or as part of, a method or system for sequencing nucleic acids (e.g., a next-generation sequencing system) to generate a plurality of sequence reads that overlap one or more gene loci within a subgenomic interval in the sample and thereby determine, e.g., gene allele sequences at a plurality of gene loci. “Next-generation sequencing” (or “NGS”) as used herein may also be referred to as “massively parallel sequencing” (or “MPS”), and refers to any sequencing method that determines the nucleotide sequence of either individual nucleic acid molecules (e.g., as in single molecule sequencing) or clonally expanded proxies for individual nucleic acid molecules in a high throughput fashion (e.g., wherein greater than 103, 104, 105 or more than 105 molecules are sequenced simultaneously).

Next-generation sequencing methods are known in the art, and are described in, e.g., Metzker, M. (2010) Nature Biotechnology Reviews 11:31-46, which is incorporated herein by reference. Other examples of sequencing methods suitable for use when implementing the methods and systems disclosed herein are described in, e.g., International Patent Application Publication No. WO 2012/092426. In some instances, the sequencing may comprise, for example, whole genome sequencing (WGS), whole exome sequencing, targeted sequencing, or direct sequencing. In some instances, sequencing may be performed using, e.g., Sanger sequencing. In some instances, the sequencing may comprise a paired-end sequencing technique that allows both ends of a fragment to be sequenced and generates high-quality, alignable sequence data for detection of, e.g., genomic rearrangements, repetitive sequence elements, gene fusions, and novel transcripts.

The disclosed methods and systems may be implemented using sequencing platforms such as the Roche 454, Illumina Solexa, ABI-SOLID, ION Torrent, Complete Genomics, Pacific Bioscience, Helicos, and/or the Polonator platform. In some instances, sequencing may comprise Illumina MiSeq sequencing. In some instances, sequencing may comprise Illumina HiSeq sequencing. In some instances, sequencing may comprise Illumina NovaSeq sequencing. Optimized methods for sequencing a large number of target genomic loci in nucleic acids extracted from a sample are described in more detail in, e.g., International Patent Application Publication No. WO 2020/236941, the entire content of which is incorporated herein by reference.

In certain instances, the disclosed methods comprise one or more of the steps of: (a) acquiring a library comprising a plurality of normal and/or tumor nucleic acid molecules from a sample; (b) simultaneously or sequentially contacting the library with one, two, three, four, five, or more than five pluralities of target capture reagents under conditions that allow hybridization of the target capture reagents to the target nucleic acid molecules, thereby providing a selected set of captured normal and/or tumor nucleic acid molecules (i.e., a library catch); (c) separating the selected subset of the nucleic acid molecules (e.g., the library catch) from the hybridization mixture, e.g., by contacting the hybridization mixture with a binding entity that allows for separation of the target capture reagent/nucleic acid molecule hybrids from the hybridization mixture, (d) sequencing the library catch to acquiring a plurality of reads (e.g., sequence reads) that overlap one or more subject intervals (e.g., one or more target sequences) from said library catch that may comprise a mutation (or alteration), e.g., a variant sequence comprising a somatic mutation or germline mutation; (e) aligning said sequence reads using an alignment method as described elsewhere herein; and/or (f) assigning a nucleotide value for a nucleotide position in the subject interval (e.g., calling a mutation using, e.g., a Bayesian method or other method described herein) from one or more sequence reads of the plurality.

In some instances, acquiring sequence reads for one or more subject intervals may comprise sequencing at least 1, at least 5, at least 10, at least 20, at least 30, at least 40, at least 50, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1,000, at least 1,250, at least 1,500, at least 1,750, at least 2,000, at least 2,250, at least 2,500, at least 2,750, at least 3,000, at least 3,500, at least 4,000, at least 4,500, or at least 5,000 loci, e.g., genomic loci, gene loci, microsatellite loci, etc. In some instances, acquiring a sequence read for one or more subject intervals may comprise sequencing a subject interval for any number of loci within the range described in this paragraph, e.g., for at least 2,850 gene loci.

In some instances, acquiring a sequence read for one or more subject intervals comprises sequencing a subject interval with a sequencing method that provides a sequence read length (or average sequence read length) of at least 20 bases, at least 30 bases, at least 40 bases, at least 50 bases, at least 60 bases, at least 70 bases, at least 80 bases, at least 90 bases, at least 100 bases, at least 120 bases, at least 140 bases, at least 160 bases, at least 180 bases, at least 200 bases, at least 220 bases, at least 240 bases, at least 260 bases, at least 280 bases, at least 300 bases, at least 320 bases, at least 340 bases, at least 360 bases, at least 380 bases, or at least 400 bases. In some instances, acquiring a sequence read for the one or more subject intervals may comprise sequencing a subject interval with a sequencing method that provides a sequence read length (or average sequence read length) of any number of bases within the range described in this paragraph, e.g., a sequence read length (or average sequence read length) of 56 bases.

In some instances, acquiring a sequence read for one or more subject intervals may comprise sequencing with at least 100× or more coverage (or depth) on average. In some instances, acquiring a sequence read for one or more subject intervals may comprise sequencing with at least 100×, at least 150×, at least 200×, at least 250×, at least 500×, at least 750×, at least 1,000×, at least 1,500×, at least 2,000×, at least 2,500×, at least 3,000×, at least 3,500×, at least 4,000×, at least 4,500×, at least 5,000×, at least 5,500×, or at least 6,000× or more coverage (or depth) on average. In some instances, acquiring a sequence read for one or more subject intervals may comprise sequencing with an average coverage (or depth) having any value within the range of values described in this paragraph, e.g., at least 160×.

In some instances, acquiring a read for the one or more subject intervals comprises sequencing with an average sequencing depth having any value ranging from at least 100× to at least 6,000× for greater than about 90%, 92%, 94%, 95%, 96%, 97%, 98%, or 99% of the gene loci sequenced. For example, in some instances acquiring a read for the subject interval comprises sequencing with an average sequencing depth of at least 125× for at least 99% of the gene loci sequenced. As another example, in some instances acquiring a read for the subject interval comprises sequencing with an average sequencing depth of at least 4,100× for at least 95% of the gene loci sequenced.

In some instances, the relative abundance of a nucleic acid species in the library can be estimated by counting the relative number of occurrences of their cognate sequences (e.g., the number of sequence reads for a given cognate sequence) in the data generated by the sequencing experiment.

In some instances, the disclosed methods and systems provide nucleotide sequences for a set of subject intervals (e.g., gene loci), as described herein. In certain instances, the sequences are provided without using a method that includes a matched normal control (e.g., a wild-type control) and/or a matched tumor control (e.g., primary versus metastatic).

In some instances, the level of sequencing depth as used herein (e.g., an X-fold level of sequencing depth) refers to the number of reads (e.g., unique reads) obtained after detection and removal of duplicate reads (e.g., PCR duplicate reads). In other instances, duplicate reads are evaluated, e.g., to support detection of copy number alteration (CNAs).

### Alignment

Alignment is the process of matching a read with a location, e.g., a genomic location or locus. In some instances, NGS reads may be aligned to a known reference sequence (e.g., a wild-type sequence). In some instances, NGS reads may be assembled de novo. Methods of sequence alignment for NGS reads are described in, e.g., Trapnell, C. and Salzberg, S. L. Nature Biotech., 2009, 27:455-457. Examples of de novo sequence assemblies are described in, e.g., Warren R., et al., Bioinformatics, 2007, 23:500-501; Butler, J. et al., Genome Res., 2008, 18:810-820; and Zerbino, D. R. and Birney, E., Genome Res., 2008, 18:821-829. Optimization of sequence alignment is described in the art, e.g., as set out in International Patent Application Publication No. WO 2012/092426. Additional description of sequence alignment methods is provided in, e.g., International Patent Application Publication No. WO 2020/236941, the entire content of which is incorporated herein by reference.

Misalignment (e.g., the placement of base-pairs from a short read at incorrect locations in the genome), e.g., misalignment of reads due to sequence context (e.g., the presence of repetitive sequence) around an actual cancer mutation can lead to reduction in sensitivity of mutation detection, can lead to a reduction in sensitivity of mutation detection, as reads for the alternate allele may be shifted off the histogram peak of alternate allele reads. Other examples of sequence context that may cause misalignment include short-tandem repeats, interspersed repeats, low complexity regions, insertions-deletions (indels), and paralogs. If the problematic sequence context occurs where no actual mutation is present, misalignment may introduce artifactual reads of “mutated” alleles by placing reads of actual reference genome base sequences at the wrong location. Because mutation-calling algorithms for multigene analysis should be sensitive to even low-abundance mutations, sequence misalignments may increase false positive discovery rates and/or reduce specificity.

In some instances, the methods and systems disclosed herein may integrate the use of multiple, individually-tuned, alignment methods or algorithms to optimize base-calling performance in sequencing methods, particularly in methods that rely on massively parallel sequencing (MPS) of a large number of diverse genetic events at a large number of diverse genomic loci. In some instances, the disclosed methods and systems may comprise the use of one or more global alignment algorithms. In some instances, the disclosed methods and systems may comprise the use of one or more local alignment algorithms. Examples of alignment algorithms that may be used include, but are not limited to, the Burrows-Wheeler Alignment (BWA) software bundle (see, e.g., Li, et al. (2009), “Fast and Accurate Short Read Alignment with Burrows-Wheeler Transform”, Bioinformatics 25:1754-60; Li, et al. (2010), Fast and Accurate Long-Read Alignment with Burrows-Wheeler Transform”, Bioinformatics epub. PMID: 20080505), the Smith-Waterman algorithm (see, e.g., Smith, et al. (1981), “Identification of Common Molecular Subsequences”, J. Molecular Biology 147 (1): 195-197), the Striped Smith-Waterman algorithm (see, e.g., Farrar (2007), “Striped Smith-Waterman Speeds Database Searches Six Times Over Other SIMD Implementations”, Bioinformatics 23 (2): 156-161), the Needleman-Wunsch algorithm (Needleman, et al. (1970) “A General Method Applicable to the Search for Similarities in the Amino Acid Sequence of Two Proteins”, J. Molecular Biology 48 (3): 443-53), or any combination thereof.

In some instances, the methods and systems disclosed herein may also comprise the use of a sequence assembly algorithm, e.g., the Arachne sequence assembly algorithm (see, e.g., Batzoglou, et al. (2002), “ARACHNE: A Whole-Genome Shotgun Assembler”, Genome Res. 12:177-189).

In some instances, the alignment method used to analyze sequence reads is not individually customized or tuned for detection of different variants (e.g., point mutations, insertions, deletions, and the like) at different genomic loci. In some instances, different alignment methods are used to analyze reads that are individually customized or tuned for detection of at least a subset of the different variants detected at different genomic loci. In some instances, different alignment methods are used to analyze reads that are individually customized or tuned to detect each different variant at different genomic loci. In some instances, tuning can be a function of one or more of: (i) the genetic locus (e.g., gene loci, microsatellite locus, or other subject interval) being sequenced, (ii) the tumor type associated with the sample, (iii) the variant being sequenced, or (iv) a characteristic of the sample or the subject. The selection or use of alignment conditions that are individually tuned to a number of specific subject intervals to be sequenced allows optimization of speed, sensitivity, and specificity. The method is particularly effective when the alignment of reads for a relatively large number of diverse subject intervals are optimized. In some instances, the method includes the use of an alignment method optimized for rearrangements in combination with other alignment methods optimized for subject intervals not associated with rearrangements.

In some instances, the methods disclosed herein further comprise selecting or using an alignment method for analyzing, e.g., aligning, a sequence read, wherein said alignment method is a function of, is selected responsive to, or is optimized for, one or more of: (i) tumor type, e.g., the tumor type in the sample; (ii) the location (e.g., a gene locus) of the subject interval being sequenced; (iii) the type of variant (e.g., a point mutation, insertion, deletion, substitution, copy number variation (CNV), rearrangement, or fusion) in the subject interval being sequenced; (iv) the site (e.g., nucleotide position) being analyzed; (v) the type of sample (e.g., a sample described herein); and/or (vi) adjacent sequence(s) in or near the subject interval being evaluated (e.g., according to the expected propensity thereof for misalignment of the subject interval due to, e.g., the presence of repeated sequences in or near the subject interval).

In some instances, the methods disclosed herein allow for the rapid and efficient alignment of troublesome reads, e.g., a read having a rearrangement. Thus, in some instances where a read for a subject interval comprises a nucleotide position with a rearrangement, e.g., a translocation, the method can comprise using an alignment method that is appropriately tuned and that includes: (i) selecting a rearrangement reference sequence for alignment with a read, wherein said rearrangement reference sequence aligns with a rearrangement (in some instances, the reference sequence is not identical to the genomic rearrangement); and (ii) comparing, e.g., aligning, a read with said rearrangement reference sequence.

In some instances, alternative methods may be used to align troublesome reads. These methods are particularly effective when the alignment of reads for a relatively large number of diverse subject intervals is optimized. By way of example, a method of analyzing a sample can comprise: (i) performing a comparison (e.g., an alignment comparison) of a read using a first set of parameters (e.g., using a first mapping algorithm, or by comparison with a first reference sequence), and determining if said read meets a first alignment criterion (e.g., the read can be aligned with said first reference sequence, e.g., with less than a specific number of mismatches); (ii) if said read fails to meet the first alignment criterion, performing a second alignment comparison using a second set of parameters, (e.g., using a second mapping algorithm, or by comparison with a second reference sequence); and (iii) optionally, determining if said read meets said second criterion (e.g., the read can be aligned with said second reference sequence, e.g., with less than a specific number of mismatches), wherein said second set of parameters comprises use of, e.g., said second reference sequence, which, compared with said first set of parameters, is more likely to result in an alignment with a read for a variant (e.g., a rearrangement, insertion, deletion, or translocation).

In some instances, the alignment of sequence reads in the disclosed methods may be combined with a mutation calling method as described elsewhere herein. As discussed herein, reduced sensitivity for detecting actual mutations may be addressed by evaluating the quality of alignments (manually or in an automated fashion) around expected mutation sites in the genes or genomic loci (e.g., gene loci) being analyzed. In some instances, the sites to be evaluated can be obtained from databases of the human genome (e.g., the HG19 human reference genome) or cancer mutations (e.g., COSMIC). Regions that are identified as problematic can be remedied with the use of an algorithm selected to give better performance in the relevant sequence context, e.g., by alignment optimization (or re-alignment) using slower, but more accurate alignment algorithms such as Smith-Waterman alignment. In cases where general alignment algorithms cannot remedy the problem, customized alignment approaches may be created by, e.g., adjustment of maximum difference mismatch penalty parameters for genes with a high likelihood of containing substitutions; adjusting specific mismatch penalty parameters based on specific mutation types that are common in certain tumor types (e.g. C→T in melanoma); or adjusting specific mismatch penalty parameters based on specific mutation types that are common in certain sample types (e.g. substitutions that are common in FFPE).

Reduced specificity (increased false positive rate) in the evaluated subject intervals due to misalignment can be assessed by manual or automated examination of all mutation calls in the sequencing data. Those regions found to be prone to spurious mutation calls due to misalignment can be subjected to alignment remedies as discussed above. In cases where no algorithmic remedy is found possible, “mutations” from the problem regions can be classified or screened out from the panel of targeted loci.

### Mutation Calling

Base calling refers to the raw output of a sequencing device, e.g., the determined sequence of nucleotides in an oligonucleotide molecule. Mutation calling refers to the process of selecting a nucleotide value, e.g., A, G, T, or C, for a given nucleotide position being sequenced. Typically, the sequence reads (or base calling) for a position will provide more than one value, e.g., some reads will indicate a T and some will indicate a G. Mutation calling is the process of assigning a correct nucleotide value, e.g., one of those values, to the sequence. Although it is referred to as “mutation” calling, it can be applied to assign a nucleotide value to any nucleotide position, e.g., positions corresponding to mutant alleles, wild-type alleles, alleles that have not been characterized as either mutant or wild-type, or to positions not characterized by variability.

In some instances, the disclosed methods may comprise the use of customized or tuned mutation calling algorithms or parameters thereof to optimize performance when applied to sequencing data, particularly in methods that rely on massively parallel sequencing (MPS) of a large number of diverse genetic events at a large number of diverse genomic loci (e.g., gene loci, microsatellite regions, etc.) in samples, e.g., samples from a subject having cancer. Optimization of mutation calling is described in the art, e.g., as set out in International Patent Application Publication No. WO 2012/092426.

Methods for mutation calling can include one or more of the following: making independent calls based on the information at each position in the reference sequence (e.g., examining the sequence reads; examining the base calls and quality scores; calculating the probability of observed bases and quality scores given a potential genotype; and assigning genotypes (e.g., using Bayes' rule)); removing false positives (e.g., using depth thresholds to reject SNPs with read depth much lower or higher than expected; local realignment to remove false positives due to small indels); and performing linkage disequilibrium (LD)/imputation-based analysis to refine the calls.

Equations used to calculate the genotype likelihood associated with a specific genotype and position are described in, e.g., Li, H. and Durbin, R. Bioinformatics, 2010; 26 (5): 589-95. The prior expectation for a particular mutation in a certain cancer type can be used when evaluating samples from that cancer type. Such likelihood can be derived from public databases of cancer mutations, e.g., Catalogue of Somatic Mutation in Cancer (COSMIC), HGMD (Human Gene Mutation Database), The SNP Consortium, Breast Cancer Mutation Data Base (BIC), and Breast Cancer Gene Database (BCGD).

Examples of LD/imputation based analysis are described in, e.g., Browning, B. L. and Yu, Z. Am. J. Hum. Genet. 2009, 85 (6): 847-61. Examples of low-coverage SNP calling methods are described in, e.g., Li, Y., et al., Annu. Rev. Genomics Hum. Genet. 2009, 10:387-406.

After alignment, detection of substitutions can be performed using a mutation calling method (e.g., a Bayesian mutation calling method) which is applied to each base in each of the subject intervals, e.g., exons of a gene or other locus to be evaluated, where presence of alternate alleles is observed. This method will compare the probability of observing the read data in the presence of a mutation with the probability of observing the read data in the presence of base-calling error alone. Mutations can be called if this comparison is sufficiently strongly supportive of the presence of a mutation.

An advantage of a Bayesian mutation-detection approach is that the comparison of the probability of the presence of a mutation with the probability of base-calling error alone can be weighted by a prior expectation of the presence of a mutation at the site. If some reads of an alternate allele are observed at a frequently mutated site for the given cancer type, then presence of a mutation may be confidently called even if the amount of evidence of mutation does not meet the usual thresholds. This flexibility can then be used to increase detection sensitivity for even rarer mutations/lower purity samples, or to make the test more robust to decreases in read coverage. The likelihood of a random base-pair in the genome being mutated in cancer is ˜le-6. The likelihood of specific mutations occurring at many sites in, for example, a typical multigenic cancer genome panel can be orders of magnitude higher. These likelihoods can be derived from public databases of cancer mutations (e.g., COSMIC).

Indel calling is a process of finding bases in the sequencing data that differ from the reference sequence by insertion or deletion, typically including an associated confidence score or statistical evidence metric. Methods of indel calling can include the steps of identifying candidate indels, calculating genotype likelihood through local re-alignment, and performing LD-based genotype inference and calling. Typically, a Bayesian approach is used to obtain potential indel candidates, and then these candidates are tested together with the reference sequence in a Bayesian framework.

Algorithms to generate candidate indels are described in, e.g., McKenna, A., et al., Genome Res. 2010; 20 (9): 1297-303; Ye, K., et al., Bioinformatics, 2009; 25 (21): 2865-71; Lunter, G., and Goodson, M., Genome Res. 2011; 21 (6): 936-9; and Li, H., et al. (2009), Bioinformatics 25 (16): 2078-9.

Methods for generating indel calls and individual-level genotype likelihoods include, e.g., the Dindel algorithm (Albers, C. A., et al., Genome Res. 2011; 21 (6): 961-73). For example, the Bayesian EM algorithm can be used to analyze the reads, make initial indel calls, and generate genotype likelihoods for each candidate indel, followed by imputation of genotypes using, e.g., QCALL (Le S. Q. and Durbin R. Genome Res. 2011; 21 (6): 952-60). Parameters, such as prior expectations of observing the indel can be adjusted (e.g., increased or decreased), based on the size or location of the indels.

Methods have been developed that address limited deviations from allele frequencies of 50% or 100% for the analysis of cancer DNA. (see, e.g., SNVMix-Bioinformatics. 2010 Mar. 15; 26 (6): 730-736.) Methods disclosed herein, however, allow consideration of the possibility of the presence of a mutant allele at frequencies (or allele fractions) ranging from 1% to 100% (i.e., allele fractions ranging from 0.01 to 1.0), and especially at levels lower than 50%. This approach is particularly important for the detection of mutations in, for example, low-purity FFPE samples of natural (multi-clonal) tumor DNA.

In some instances, the mutation calling method used to analyze sequence reads is not individually customized or fine-tuned for detection of different mutations at different genomic loci. In some instances, different mutation calling methods are used that are individually customized or fine-tuned for at least a subset of the different mutations detected at different genomic loci. In some instances, different mutation calling methods are used that are individually customized or fine-tuned for each different mutant detected at each different genomic loci. The customization or tuning can be based on one or more of the factors described herein, e.g., the type of cancer in a sample, the gene or locus in which the subject interval to be sequenced is located, or the variant to be sequenced. This selection or use of mutation calling methods individually customized or fine-tuned for a number of subject intervals to be sequenced allows for optimization of speed, sensitivity and specificity of mutation calling.

In some instances, a nucleotide value is assigned for a nucleotide position in each of X unique subject intervals using a unique mutation calling method, and X is at least 2, at least 3, at least 4, at least 5, at least 10, at least 15, at least 20, at least 30, at least 40, at least 50, at least 60, at least 70, at least 80, at least 90, at least 100, at least 200, at least 300, at least 400, at least 500, at least 1000, at least 1500, at least 2000, at least 2500, at least 3000, at least 3500, at least 4000, at least 4500, at least 5000, or greater. The calling methods can differ, and thereby be unique, e.g., by relying on different Bayesian prior values.

In some instances, assigning said nucleotide value is a function of a value which is or represents the prior (e.g., literature) expectation of observing a read showing a variant, e.g., a mutation, at said nucleotide position in a tumor of type.

In some instances, the method comprises assigning a nucleotide value (e.g., calling a mutation) for at least 10, 20, 40, 50, 60, 70, 80, 90, 100, 200, 300, 400, 500, 600, 700, 800, 900, or 1,000 nucleotide positions, wherein each assignment is a function of a unique value (as opposed to the value for the other assignments) which is or represents the prior (e.g., literature) expectation of observing a read showing a variant, e.g., a mutation, at said nucleotide position in a tumor of type.

In some instances, assigning said nucleotide value is a function of a set of values which represent the probabilities of observing a read showing said variant at said nucleotide position if the variant is present in the sample at a specified frequency (e.g., 1%, 5%, 10%, etc.) and/or if the variant is absent (e.g., observed in the reads due to base-calling error alone).

In some instances, the mutation calling methods described herein can include the following: (a) acquiring, for a nucleotide position in each of said X subject intervals: (i) a first value which is or represents the prior (e.g., literature) expectation of observing a read showing a variant, e.g., a mutation, at said nucleotide position in a tumor of type X; and (ii) a second set of values which represent the probabilities of observing a read showing said variant at said nucleotide position if the variant is present in the sample at a frequency (e.g., 1%, 5%, 10%, etc.) and/or if the variant is absent (e.g., observed in the reads due to base-calling error alone); and (b) responsive to said values, assigning a nucleotide value (e.g., calling a mutation) from said reads for each of said nucleotide positions by weighing, e.g., by a Bayesian method described herein, the comparison among the values in the second set using the first value (e.g., computing the posterior probability of the presence of a mutation), thereby analyzing said sample.

Additional description of mutation calling methods is provided in, e.g., International Patent Application Publication No. WO 2020/236941, the entire content of which is incorporated herein by reference.

### Systems

Also disclosed herein are systems designed to implement any of the disclosed methods for predicting an origin of a plurality of reads corresponding to an alteration of interest in a sample from a subject. The systems may comprise, e.g., one or more processors, and a memory unit communicatively coupled to the one or more processors and configured to store instructions that, when executed by the one or more processors, cause the system to: receive, using one or more processors, sequence read data associated with the sample; select, using the one or more processors, a plurality of reads from the sequence read data based on the alteration; determine, using the one or more processors, at least one feature characterizing the selected plurality of reads; input, using the one or more processors, the at least one feature characterizing the selected plurality of reads into a statistical model; generate, using the one or more processors, a score indicative of the origin of the alteration by the statistical model; and predict, using the one or more processors, the origin of the alteration in the sample by comparing the score and one or more predefined thresholds.

In some instances, the disclosed systems may further comprise a sequencer, e.g., a next generation sequencer (also referred to as a massively parallel sequencer). Examples of next generation (or massively parallel) sequencing platforms include, but are not limited to, Roche/454's Genome Sequencer (GS) FLX system, Illumina/Solexa's Genome Analyzer (GA), Illumina's HiSeq® 2500, HiSeq® 3000, HiSeq® 4000 and NovaSeq® 6000 sequencing systems, Life/APG's Support Oligonucleotide Ligation Detection (SOLID) system, Polonator's G.007 system, Helicos BioSciences' HeliScope Gene Sequencing system, ThermoFisher Scientific's Ion Torrent Genexus system, or Pacific Biosciences' PacBio® RS system.

In some instances, the disclosed systems may be used for predicting an origin of a plurality of reads corresponding to an alteration of interest in a subject sample in any of a variety of samples as described herein (e.g., hematological sample, or liquid biopsy sample derived from the subject).

In some instances, the plurality of gene loci for which sequencing data is processed to determine predicting an origin of a plurality of reads corresponding to an alteration of interest in a subject sample may comprise at least 1, 2, 3, 4, 5, 6, 7, 8, 9, 10, 20, 30, 40, 50, 60, 70, 80, 90, 100, or more than 100 gene loci.

In some instance, the nucleic acid sequence data is acquired using a next generation sequencing technique (also referred to as a massively parallel sequencing technique) having a read-length of less than 400 bases, less than 300 bases, less than 200 bases, less than 150 bases, less than 100 bases, less than 90 bases, less than 80 bases, less than 70 bases, less than 60 bases, less than 50 bases, less than 40 bases, or less than 30 bases.

In some instances, the determination of predicting an origin of a plurality of reads corresponding to an alteration of interest in a subject sample is used to select, initiate, adjust, or terminate a treatment for cancer in the subject (e.g., a patient) from which the sample was derived, as described elsewhere herein.

In some instances, the disclosed systems may further comprise sample processing and library preparation workstations, microplate-handling robotics, fluid dispensing systems, temperature control modules, environmental control chambers, additional data storage modules, data communication modules (e.g., Bluetooth®, WiFi, intranet, or internet communication hardware and associated software), display modules, one or more local and/or cloud-based software packages (e.g., instrument/system control software packages, sequencing data analysis software packages), etc., or any combination thereof. In some instances, the systems may comprise, or be part of, a computer system or computer network as described elsewhere herein.

### Computer Systems and Networks

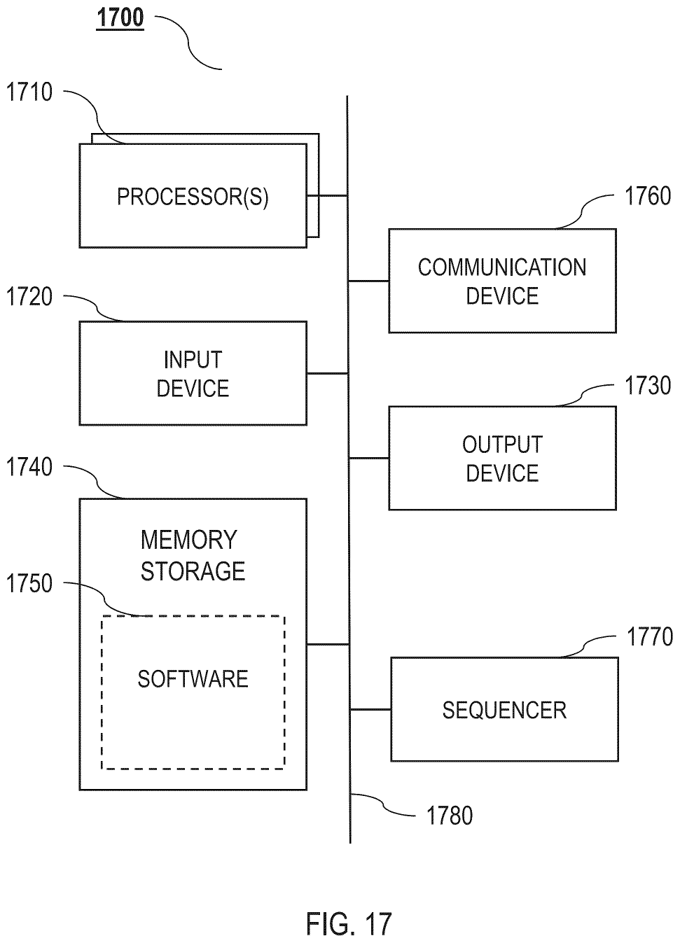

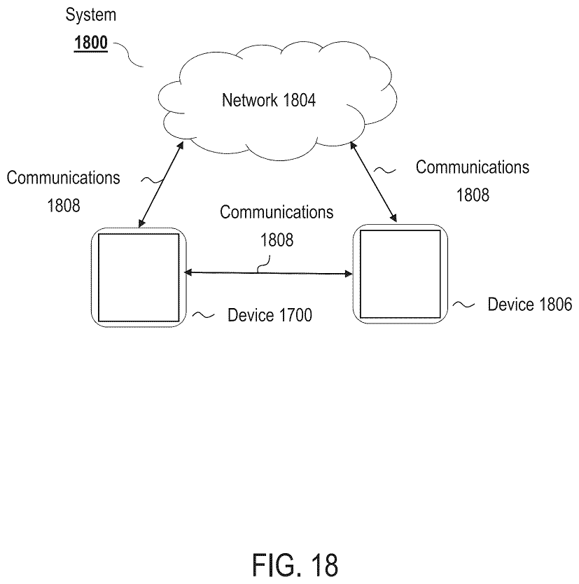

FIG. 17 illustrates an example of a computing device or system in accordance with one embodiment. Device 1700 can be a host computer connected to a network. Device 1700 can be a client computer or a server. As shown in FIG. 17, device 1700 can be any suitable type of microprocessor-based device, such as a personal computer, workstation, server or handheld computing device (portable electronic device) such as a phone or tablet. The device can include, for example, one or more processor(s) 1710, input devices 1720, output devices 1730, memory or storage devices 1740, communication devices 1760, and nucleic acid sequencers 1770. Software 1750 residing in memory or storage device 1740 may comprise, e.g., an operating system as well as software for executing the methods described herein. Input device 1720 and output device 1730 can generally correspond to those described herein, and can either be connectable or integrated with the computer.

Input device 1720 can be any suitable device that provides input, such as a touch screen, keyboard or keypad, mouse, or voice-recognition device. Output device 1730 can be any suitable device that provides output, such as a touch screen, haptics device, or speaker.

Storage 1740 can be any suitable device that provides storage (e.g., an electrical, magnetic or optical memory including a RAM (volatile and non-volatile), cache, hard drive, or removable storage disk). Communication device 1760 can include any suitable device capable of transmitting and receiving signals over a network, such as a network interface chip or device. The components of the computer can be connected in any suitable manner, such as via a wired media (e.g., a physical system bus 1780, Ethernet connection, or any other wire transfer technology) or wirelessly (e.g., Bluetooth®, Wi-Fi®, or any other wireless technology).

Software module 1750, which can be stored as executable instructions in storage 1740 and executed by processor(s) 1710, can include, for example, an operating system and/or the processes that embody the functionality of the methods of the present disclosure (e.g., as embodied in the devices as described herein).

Software module 1750 can also be stored and/or transported within any non-transitory computer-readable storage medium for use by or in connection with an instruction execution system, apparatus, or device, such as those described herein, that can fetch instructions associated with the software from the instruction execution system, apparatus, or device and execute the instructions. In the context of this disclosure, a computer-readable storage medium can be any medium, such as storage 1740, that can contain or store processes for use by or in connection with an instruction execution system, apparatus, or device. Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art will appreciate that the above processes may be routines or modules within other processes.

Software module 1750 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

Device 1700 may be connected to a network (e.g., network 1804, as shown in FIG. 13 and/or described below), which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 1700 can be implemented using any operating system, e.g., an operating system suitable for operating on the network. Software module 1750 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example. In some embodiments, the operating system is executed by one or more processors, e.g., processor(s) 1710.

Device 1700 can further include a sequencer 1770, which can be any suitable nucleic acid sequencing instrument.

FIG. 13 illustrates an example of a computing system in accordance with one embodiment. In system 1800, device 1700 (e.g., as described above and illustrated in FIG. 17) is connected to network 1804, which is also connected to device 1806. In some embodiments, device 1806 is a sequencer. Exemplary sequencers can include, without limitation, Roche/454's Genome Sequencer (GS) FLX System, Illumina/Solexa's Genome Analyzer (GA), Illumina's HiSeq® 2500, HiSeq® 3000, HiSeq® 4000 and NovaSeq® 6000 Sequencing Systems, Life/APG's Support Oligonucleotide Ligation Detection (SOLID) system, Polonator's G.007 system, Helicos BioSciences' HeliScope Gene Sequencing system, or Pacific Biosciences' PacBio® RS system.

Devices 1700 and 1806 may communicate, e.g., using suitable communication interfaces via network 1804, such as a Local Area Network (LAN), Virtual Private Network (VPN), or the Internet. In some embodiments, network 1804 can be, for example, the Internet, an intranet, a virtual private network, a cloud network, a wired network, or a wireless network. Devices 1700 and 1806 may communicate, in part or in whole, via wireless or hardwired communications, such as Ethernet, IEEE 802.11b wireless, or the like. Additionally, devices 1700 and 1806 may communicate, e.g., using suitable communication interfaces, via a second network, such as a mobile/cellular network. Communication between devices 1700 and 1806 may further include or communicate with various servers such as a mail server, mobile server, media server, telephone server, and the like. In some embodiments, Devices 1700 and 1806 can communicate directly (instead of, or in addition to, communicating via network 1804), e.g., via wireless or hardwired communications, such as Ethernet, IEEE 802.11b wireless, or the like. In some embodiments, devices 1700 and 1806 communicate via communications 1808, which can be a direct connection or can occur via a network (e.g., network 1804).

One or all of devices 1700 and 1806 generally include logic (e.g., http web server logic) or are programmed to format data, accessed from local or remote databases or other sources of data and content, for providing and/or receiving information via network 1804 according to various examples described herein.

### EXAMPLES

As noted above, liquid biopsy tests provide healthcare providers with a less-invasive method of obtaining and analyzing patient sample for detecting potentially cancerous cells in order to diagnose and treat patient. Liquid biopsy tests examine cell free DNA (cfDNA) and can detect multiple categories of alterations, including germline alleles, somatic mutations from tumor cells, and alterations that drive clonal hematopoiesis (CH). Due to overlap in the underlying biological processes, the liquid biopsy tests can misclassify CH-derived alterations as tumor-derived alterations, which can inflate the number of tumor-derived alterations detected in the sample. A healthcare provider that recommends a therapy based on the results of a test that does not account for the potential misclassification of CH-derived alterations as tumor-derived alterations runs the risk of prescribing an ineffective treatment while exposing the patient to the risks and adverse side-effects associated with the treatment. Accordingly, healthcare providers and patients alike would thus benefit from having access to a more accurate cfDNA testing to improve clinical management for the patient's solid tumor.

Embodiments of the present disclosure consider the length distribution of cfDNA sequence reads to determine whether an alteration is tumor-derived or CH-derived.

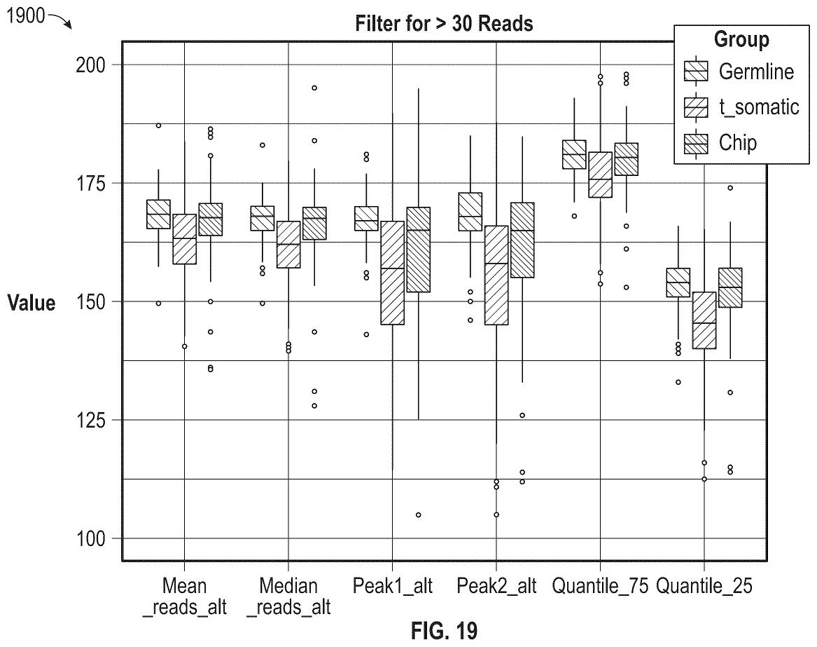

FIG. 19 is a plot 1900 that shows the differences in various fragmentomic characteristics for germline (e.g., wild type) cells, tumor-derived cells, and CH-derived cells. As shown in the figure, samples with a higher concentration of ctDNA corresponds to fragmentomics characteristics associated with a shorter fragment length. Plot 1900 illustrates that for the mean fragment length, median fragment length, first peak of the fragment length (e.g., mode), second peak of the fragment length, 75th quartile fragment length, and 25th quartile fragment length is associated with a shorter length for the tumor-derived cells compared to the germline and CH-derived cells. Accordingly, based on this analysis the inventors determined that fragmentomic characteristics could be used to distinguish CH-derived alterations from tumor-derived alterations to improve the accuracy of liquid biopsy tests.

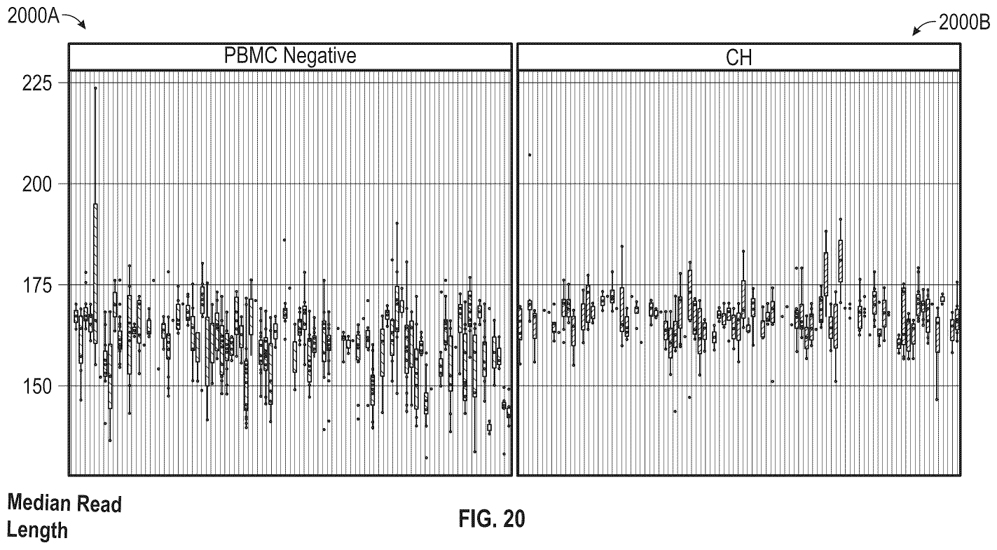

FIG. 20 is a side-by-side box plot 2000 that shows the difference between the distribution of median fragment lengths for a sample that includes tumor-derived cells 2000A and a sample that includes CH-derived cells 2000B. As shown in the figure, while the tumor-derived cells 2000A may include some fragments (e.g., box plots) that have a median fragment length that is higher than the median fragment length of one or more of the CH-derived fragments, the median fragment length for the tumor-derived samples is, more often than not, less than the median fragment length for the CH-derived samples. Accordingly, the inventors determined that fragment characteristics for the sample as opposed to individual sequencing reads and/or fragments should be used to distinguish CH-derived alterations from tumor-derived alterations.

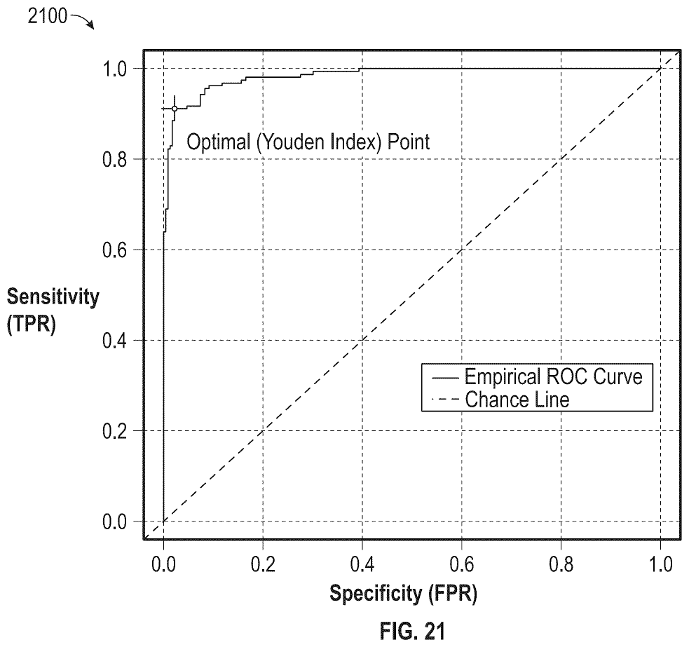

FIG. 21 is a plot 2100 that shows a receiver operating characteristic (ROC) curve for a convolutional neural network model to predict whether a plurality of reads from a sample is tumor derived or CH-derived based on fragmentomic and other characteristics. As shown in the figure, the ROC curve is based on prediction results in validation (or held-out) sets of a 5-fold cross-validation. In some examples, other validation methods can be used. As shown in the figure, the area under the curve of plot 2100 is 97.3%. At the cutpoint that maximizes the Youden index (e.g., statistic that illustrates the performance of the diagnostic system), the sensitivity (e.g., probability of predicting a true CH alteration as CH) is 94.9% and the specificity (e.g., probability of predicting a true somatic alteration as somatic) is 91.7%.

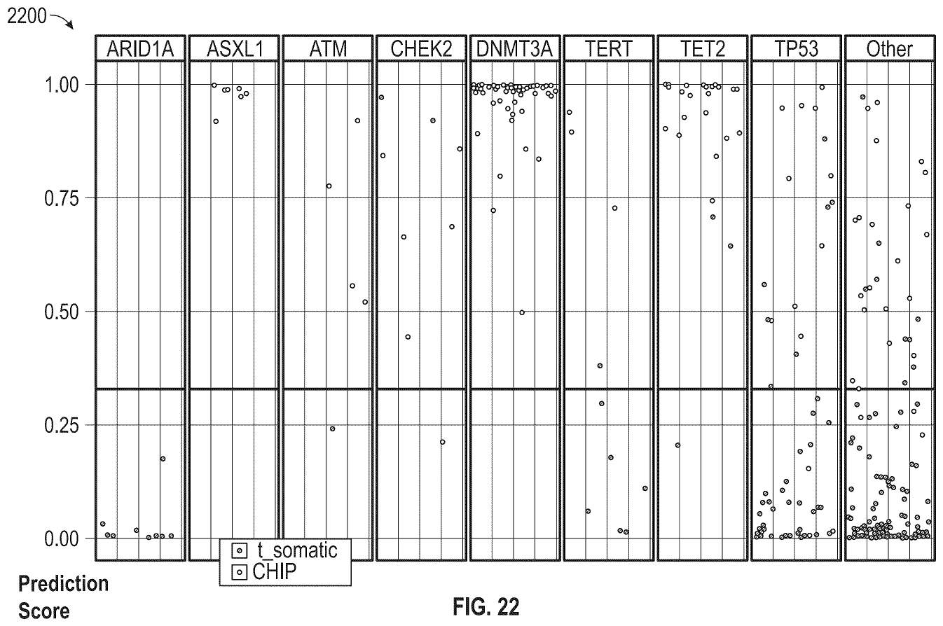

FIG. 22 is plot 2200 that shows exemplary prediction scores for a plurality of patient samples across a plurality of genes for tumor-derived fragments and CH-derived fragments. Each dot in the figure represents a prediction score for a patient sample for a particular gene (e.g., ARID1A, ASXL1, ATM, CHEK2, DNMT3A, TERT, TET2, TP53). As shown in the figure, in this example the threshold is set to 0.733, such that alterations with a score above the threshold would be predicted to be CH-derived and alterations below the threshold would be predicted to be tumor-derived. Based on the figure, there are very few alterations that are tumor-derived that are located above the threshold and few alterations that are CH-derived that are located below the threshold. Accordingly, the 0.733 threshold effectively predicts the tumor-derived reads from the CH-derived reads. This threshold is exemplary and more than one threshold and/or different thresholds can be used without departing from the scope of this disclosure.

Examples in accordance with this disclosure can be used to predict tumor somatic versus clonal hematopoiesis origin for short variants in liquid assay. As discussed above, a common challenge presented by emerging liquid biopsy technology is to distinguish somatic variants originated in CH from those in tumor. While sequencing of genomic DNA from matched peripheral blood mononuclear cells (PBMC) can effectively identify CH variants, its wide application still remains prohibitively expensive. Computational-only solutions are challenging due to shared bioinformatic presentations in both CH and somatic variants such as low allele frequency. Development of such algorithm with clinically meaningful accuracy will have extensive utility in cancer care.

In one example, a statistical model was developed according to embodiments of this disclosure. This exemplary statistical model was developed using paired plasma-buffy coat samples from 754 pan-cancer patients. The samples were sequenced using a liquid sample assay and randomized into a training (532 samples) and a test set (222 samples). Ground truth (tumor somatic vs. CH) for short variants, including base substitutions and short indels, was determined by comparing variant calls in plasma and in PBMC. The exemplary statistical model was developed as a deep learning prediction model using the training set data. This exemplary model incorporates genomic and clinical features as an input and returns a prediction score indicative of the origin of the alteration, (e.g., whether the alteration is from clonal hematopoiesis). The prediction score was dichotomized by a learned threshold to determine whether alteration is CH derived. After the threshold and model parameters were finalized, the model was applied to the test set to evaluate performance.

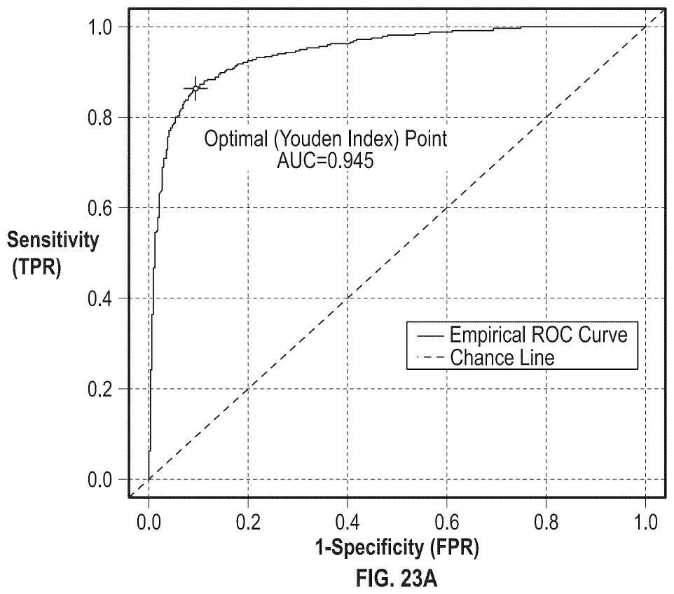

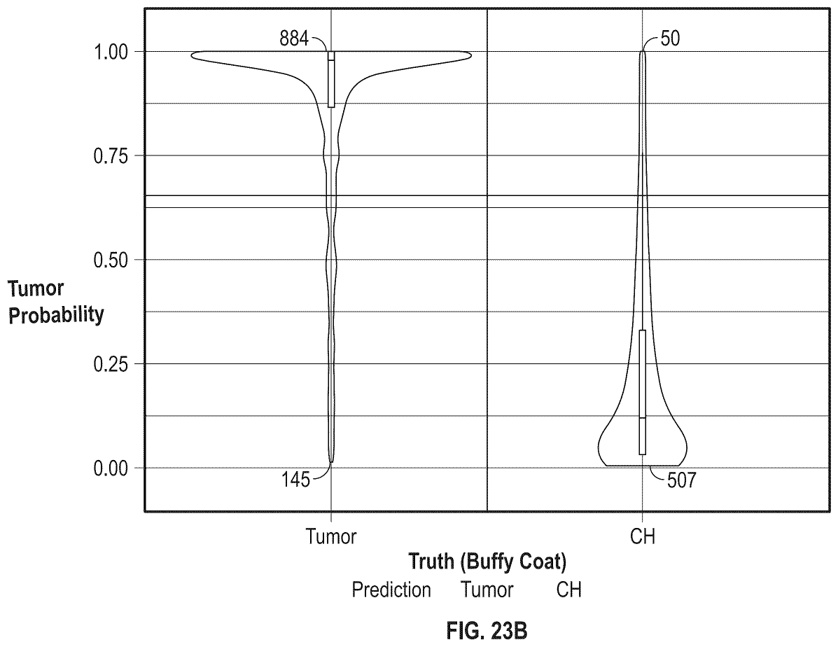

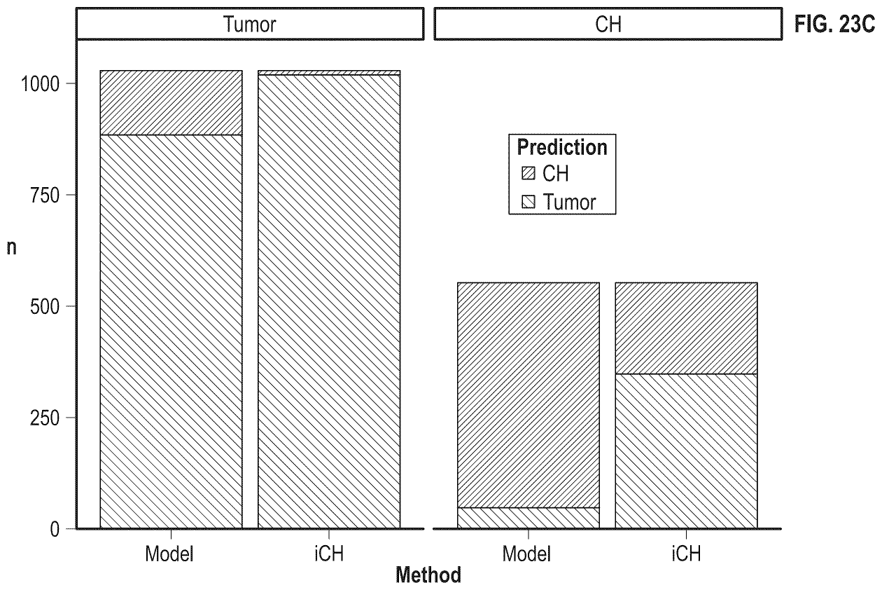

FIG. 23A illustrates an ROC curve for this exemplary model trained on the test set comprising 222 paired samples (523 CH and 904 tumor somatic variants identified in the samples). FIG. 23B illustrates a plot that shows the overall prediction accuracy of the exemplary model. As shown in the figures, the exemplary model achieved 91% sensitivity (probability of predicting true CH variants as CH) and 88% specificity (probability of predicting true tumor somatic variants as tumor somatic) based on the test set. FIG. 23C illustrates a benchmark with inferred CH (iCH), a computational method that predicts CH status based on a list of common CH genes and alterations. As shown in the figure, a model developed in accordance with embodiments of the present disclosure identified a greater fraction of true tumor alterations than inferred CH. A small increase in true tumor alterations falsely called as CH is observed with this model, compared to inferred CH.

The prediction score of the exemplary model accurately identified the origin of one or more alterations (e.g., identified CH alterations) in challenging genes such as TP53 (sensitivity=94%, specificity=90%, n=144) and ATM (sensitivity=93%, specificity=95%, n=49), which are known to harbor both tumor somatic and CH variants. Model performance was further evaluated by cancer types and variant allele frequency. CH variants were identified with 90% sensitivity and 89% specificity in non-small cell lung carcinoma (n=384), and with 87% sensitivity and 100% specificity in pancreas cancer (n=53). CH variants were identified with 92% sensitivity and 87% specificity in 1%-5% variant allele frequency (n=645), and with 94% sensitivity and 94% specificity in 5%-10% variant allele frequency (n=189). Of note, a well-known oncogenic mutation in ATM was predicted as CH in a prostate cancer patient, which would prevent ineffective administration of PARP inhibitors. Multiple plasma specimens also gained more accurate bTMB and tumor fraction estimation by filtering out CH variants. Tables I-III show the prediction accuracy of this model stratified by tumor type, age and VAF.

TABLE I

| | NSCLC | | PROSTATE | | BREAST | | CRC | | OTHERS | |
|---|---|---|---|---|---|---|---|---|---|---|
| | TRUE | TRUE | TRUE | TRUE | TRUE | TRUE | TRUE | TRUE | TRUE | TRUE |
| | TUMOR | CH | TUMOR | CH | TUMOR | CH | TUMOR | CH | TUMOR | CH |
| PREDICT | 253 | 12 | 86 | 9 | 85 | 4 | 162 | 9 | 298 | 16 |
| TUMOR | | | | | | | | | | |
| PREDICT | 34 | 141 | 34 | 73 | 42 | 40 | 7 | 39 | 28 | 214 |
| CH | | | | | | | | | | |

| | | | | |
|---|---|---|---|---|
| SENSITIVITY = 88% | SENSITIVITY = 72% | SENSITIVITY = 67% | SENSITIVITY = 96% | SENSITIVITY = 91% |
| SPECIFICITY = 92% | SPECIFICITY = 89% | SPECIFICITY = 91% | SPECIFICITY = 81% | SPECIFICITY = 93% |

TABLE II

| | AGE <50 | | AGE [50, 60) | | AGE [60, 70) | | AGE [70, 80) | | AGE >80 OR ND | |
|---|---|---|---|---|---|---|---|---|---|---|
| | TRUE | TRUE | TRUE | TRUE | TRUE | TRUE | TRUE | TRUE | TRUE | TRUE |
| | TUMOR | CH | TUMOR | CH | TUMOR | CH | TUMOR | CH | TUMOR | CH |
| PREDICT | 88 | 3 | 134 | 4 | 265 | 15 | 238 | 19 | 159 | 9 |
| TUMOR | | | | | | | | | | |
| PREDICT | 7 | 9 | 17 | 26 | 47 | 146 | 44 | 200 | 30 | 126 |
| CH | | | | | | | | | | |

| | | | | |
|---|---|---|---|---|
| SENSITIVITY = 93% | SENSITIVITY = 89% | SENSITIVITY = 85% | SENSITIVITY = 84% | SENSITIVITY = 84% |
| SPECIFICITY = 75% | SPECIFICITY = 87% | SPECIFICITY = 91% | SPECIFICITY = 91% | SPECIFICITY = 93% |

TABLE III

| | VAF <1% | | VAF [1, 5%) | | VAF [5, 10%) | | VAF [10, 30%) | | VAF >30% | |
|---|---|---|---|---|---|---|---|---|---|---|
| | TRUE | TRUE | TRUE | TRUE | TRUE | TRUE | TRUE | TRUE | TRUE | TRUE |
| | TUMOR | CH | TUMOR | CH | TUMOR | CH | TUMOR | CH | TUMOR | CH |
| PREDICT | 136 | 16 | 395 | 21 | 153 | 2 | 163 | 1 | 37 | 10 |
| TUMOR | | | | | | | | | | |
| PREDICT | 43 | 147 | 72 | 237 | 11 | 47 | 14 | 51 | 5 | 25 |
| CH | | | | | | | | | | |

| | | | | |
|---|---|---|---|---|
| SENSITIVITY = 76% | SENSITIVITY = 85% | SENSITIVITY = 93% | SENSITIVITY = 92% | SENSITIVITY = 88% |
| SPECIFICITY = 90% | SPECIFICITY = 92% | SPECIFICITY = 96% | SPECIFICITY = 98% | SPECIFICITY = 71% |

Accordingly, examples according to embodiments of this disclosure demonstrate that computational methods can distinguish tumor somatic from CH variants from plasma cfDNA sequencing. For instance, the statistical model described above exhibited high sensitivity and specificity in a pan-cancer cohort. In some embodiments, this statistical model may have clinical utility in CH-corrected bTMB and tumor fraction.

### EXEMPLARY IMPLEMENTATIONS

Exemplary implementations of the methods and systems described herein include:
1. A method comprising:
obtaining a first set of one or more samples from a subject;
isolating polynucleotides from the first set of one or more samples;
sequencing the isolated polynucleotides to produce sequence reads;
selecting a plurality of reads from the sequence read data based on an alteration in the first set of one or more samples;
determining at least one feature characterizing each of the selected plurality of reads;
inputting the at least one feature characterizing each of the selected plurality of reads into a trained machine learning model;
generating a score indicative of an origin of the alteration by the trained machine learning model; and
predicting the origin of the alteration in the sample by comparing the score and one or more predefined thresholds.
2. The method of clause 1, wherein the alteration includes at least one of an insertion, a deletion, or a substitution.
3. The method of any of clauses 1 to 2, further comprising:
providing a plurality of nucleic acid molecules obtained from a sample from a subject;
ligating one or more adapters onto one or more nucleic acid molecules from the plurality of nucleic acid molecules;
amplifying the one or more ligated nucleic acid molecules from the plurality of nucleic acid molecules;
capturing amplified nucleic acid molecules from the amplified nucleic acid molecules;
sequencing, by a sequencer, the captured nucleic acid molecules to obtain a plurality of sequence reads that represent the captured nucleic acid molecules;
receiving, at one or more processors, sequence read data for the plurality of sequence reads;
receiving, using one or more processors, sequence read data associated with the sample;
selecting, using the one or more processors, a plurality of reads from the sequence read data based on the alteration;
determining, using the one or more processors, at least one feature characterizing the selected plurality of reads;
inputting, using the one or more processors, the at least one feature into a statistical model;
generating, using the one or more processors, a score indicative of the origin of the alteration by the statistical model; and
predicting, using the one or more processors, the origin of the alteration in the sample by comparing the score and one or more predefined thresholds.
4. The method of any of clauses 1 to 3, wherein the statistical model is a trained statistical model or an untrained statistical model.
5. The method of any of clauses 1 to 4, wherein the statistical model is a trained machine learning model and trained by:
receiving, using the one or more processors, training data including information quantifying features related to the alteration; and
training, using the one or more processors, the machine learning model based on the training data.
6. The method of any of clauses 1 to 5, wherein the score is indicative of a probability that the alteration is derived from a solid tumor.
7. The method of any of clauses 1 to 6, wherein the score is further indicative of a probability that the alteration is derived from clonal hematopoiesis.
8. The method of any of clauses 1 to 7, wherein the at least one feature comprises at least one fragmentomic characteristic of the sample.
9. The method of any of clause 8, wherein the at least one fragmentomic characteristic of the sample comprises at least one of: an amount of the selected plurality of reads having a specified length, a mean fragment length of the selected plurality of reads, a median fragment length of the selected plurality of reads, an interquartile range of fragment lengths of the selected plurality of reads, a distribution of fragment lengths of the selected plurality of reads, one or more peaks of fragment lengths for the selected plurality of reads, a fragment length for at least one of the selected plurality of reads, a fragment end motif, a start position of a fragment for the selected plurality of reads, and an end position of a fragment for the selected plurality of reads.
10. The method of clause 9, wherein a read of the selected plurality of reads is a cfDNA fragment.
11. The method of any of clauses 1 to 10, wherein the statistical model is configured to receive one or more additional features related to the alteration.
12. The method of clause 11, wherein the one or more additional features comprise an alteration depth, an allele frequency of the selected plurality of reads, an alteration coding type, a somatic determination, a germline determination, a zygosity determination, an age of the individual, a blood tumor mutational burden score, a protein level data, a gene level data, an odds ratio describing an enrichment of the alteration in older patients over younger patients adjusted by disease group, a p-value describing a significance of an enrichment of the alteration in older patients over younger patients adjusted by disease group, an odds ratio describing an enrichment for a specific amino acid change in older patients over younger patients adjusted by disease group, a p-value describing a significance of an enrichment for a specific amino acid change in older patients over younger patients adjusted by disease group, an odds ratio describing an enrichment of the alteration in a certain cancer type compared to other cancers, a p-value describing a significance of an enrichment of the alteration in a certain cancer type compared to other cancers, an odds ratio describing an enrichment for a specific amino acid change in a certain cancer type compared to other cancers, a p-value describing a significance of an enrichment for a specific amino acid change in a certain cancer type compared to other cancers, a tumor fraction, an inferred subclonal status, a distribution of variant level germline, somatic, subclonal somatic calls, variant allele frequency changes over time, a sample level mutational signature score, a description of pathogenicity measures, or any combination thereof.
13. The method of any of clauses 1 to 12, wherein the one or more predetermined thresholds comprise a first predetermined threshold, and wherein predicting the origin of the alteration in the sample by comparing the score and one or more predefined thresholds further comprises:
comparing, using the one or more processors, the score against the first predetermined threshold; and
in accordance with a determination that the score is greater than or equal to the first predetermined threshold, determining, using the one or more processors, that the alteration is not derived from a tumor.
14. The method of clause 13, wherein the one or more predetermined thresholds comprise a second predetermined threshold, and wherein predicting, using the one or more processors, the origin of the alteration in the sample by comparing the score and one or more predefined thresholds further comprises:
comparing, using the one or more processors, the score against the second predetermined threshold; and
in accordance with a determination that the score is less than or equal to the second predetermined threshold, determining, using the one or more processors, that the alteration is derived from the tumor.
15. The method of any of clauses 13 to 14, wherein the second predetermined threshold is the same as the first predetermined threshold.
16. The method of any of clauses 1 to 15, wherein the individual is suspected of having or is determined to have cancer.
17. The method of clause 16, wherein the cancer is a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.
18. The method of clause 16, wherein the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR/MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.
19. The method of clause 18, further comprising treating the subject with an anti-cancer therapy.
20. The method of clause 19, wherein the anti-cancer therapy comprises a targeted anti-cancer therapy.
21. The method of clause 20, wherein the targeted anti-cancer therapy comprises abemaciclib (Verzenio), abiraterone acetate (Zytiga), acalabrutinib (Calquence), ado-trastuzumab emtansine (Kadcyla), afatinib dimaleate (Gilotrif), aldesleukin (Proleukin), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), copanlisib hydrochloride (Aliqopa), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), iobenguane I131 (Azedra), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), panobinostat (Farydak), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sipuleucel-T (Provenge), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.
22. The method of any of clauses 1 to 21, further comprising obtaining the sample from the subject.
23. The method of any of clauses 1 to 22, wherein the sample comprises a tissue biopsy sample, a liquid biopsy sample, or a normal control.
24. The method of clause 23, wherein the sample is a liquid biopsy sample and comprises blood, plasma, cerebrospinal fluid, sputum, stool, urine, or saliva.
25. The method of clause 23, wherein the sample is a liquid biopsy sample and comprises circulating tumor cells (CTCs).
26. The method of clause 23, wherein the sample is a liquid biopsy sample and comprises cell-free DNA (cfDNA), circulating tumor DNA (ctDNA), or any combination thereof.
27. The method of any of clauses 1 to 26, wherein the plurality of nucleic acid molecules comprises a mixture of tumor nucleic acid molecules and non-tumor nucleic acid molecules.
28. The method of clause 27, wherein the tumor nucleic acid molecules are derived from a tumor portion of a heterogeneous tissue biopsy sample, and the non-tumor nucleic acid molecules are derived from a normal portion of the heterogeneous tissue biopsy sample.
29. The method of clause 27, wherein the sample comprises a liquid biopsy sample, and wherein the tumor nucleic acid molecules are derived from a circulating tumor DNA (ctDNA) fraction of the liquid biopsy sample, and the non-tumor nucleic acid molecules are derived from a non-tumor, cell-free DNA (cfDNA) fraction of the liquid biopsy sample.
30. The method of any of clauses 1 to 29, wherein the one or more adapters comprise amplification primers, flow cell adaptor sequences, substrate adapter sequences, or sample index sequences.
31. The method of any of clauses 1 to 30, wherein the captured nucleic acid molecules are captured from the amplified nucleic acid molecules by hybridization to one or more bait molecules.
32. The method of clause 31, wherein the one or more bait molecules comprise one or more nucleic acid molecules, each comprising a region that is complementary to a region of a captured nucleic acid molecule.
33. The method of any of clauses 1 to 32, wherein amplifying nucleic acid molecules comprises performing a polymerase chain reaction (PCR) amplification technique, a non-PCR amplification technique, or an isothermal amplification technique.
34. The method of any of clauses 1 to 33, wherein the sequencing comprises use of a massively parallel sequencing (MPS) technique, whole genome sequencing (WGS), whole exome sequencing, targeted sequencing, direct sequencing, or Sanger sequencing technique.
35. The method of clause 34, wherein the sequencing comprises massively parallel sequencing, and the massively parallel sequencing technique comprises next generation sequencing (NGS).
36. The method of any of clauses 1 to 35, wherein the sequencer comprises a next generation sequencer.
37. The method of any of clauses 1 to 36, wherein one or more of the plurality of sequencing reads overlap one or more gene loci within one or more subgenomic intervals in the sample.
38. The method of clause 37, wherein the one or more gene loci comprises between 10 and 20 loci, between 10 and 40 loci, between 10 and 60 loci, between 10 and 80 loci, between 10 and 100 loci, between 10 and 150 loci, between 10 and 200 loci, between 10 and 250 loci, between 10 and 300 loci, between 10 and 350 loci, between 10 and 400 loci, between 10 and 450 loci, between 10 and 500 loci, between 20 and 40 loci, between 20 and 60 loci, between 20 and 80 loci, between 20 and 100 loci, between 20 and 150 loci, between 20 and 200 loci, between 20 and 250 loci, between 20 and 300 loci, between 20 and 350 loci, between 20 and 400 loci, between 20 and 500 loci, between 40 and 60 loci, between 40 and 80 loci, between 40 and 100 loci, between 40 and 150 loci, between 40 and 200 loci, between 40 and 250 loci, between 40 and 300 loci, between 40 and 350 loci, between 40 and 400 loci, between 40 and 500 loci, between 60 and 80 loci, between 60 and 100 loci, between 60 and 150 loci, between 60 and 200 loci, between 60 and 250 loci, between 60 and 300 loci, between 60 and 350 loci, between 60 and 400 loci, between 60 and 500 loci, between 80 and 100 loci, between 80 and 150 loci, between 80 and 200 loci, between 80 and 250 loci, between 80 and 300 loci, between 80 and 350 loci, between 80 and 400 loci, between 80 and 500 loci, between 100 and 150 loci, between 100 and 200 loci, between 100 and 250 loci, between 100 and 300 loci, between 100 and 350 loci, between 100 and 400 loci, between 100 and 500 loci, between 150 and 200 loci, between 150 and 250 loci, between 150 and 300 loci, between 150 and 350 loci, between 150 and 400 loci, between 150 and 500 loci, between 200 and 250 loci, between 200 and 300 loci, between 200 and 350 loci, between 200 and 400 loci, between 200 and 500 loci, between 250 and 300 loci, between 250 and 350 loci, between 250 and 400 loci, between 250 and 500 loci, between 300 and 350 loci, between 300 and 400 loci, between 300 and 500 loci, between 350 and 400 loci, between 350 and 500 loci, or between 400 and 500 loci.
39. The method of any of clauses 37 to 38, wherein the one or more gene loci comprise ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOTIL, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MSTIR, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCDILG2, PDGFRA, PDGFRB, PDK1, PIK3C2B, PIK3C2G, PIK3CA, PIK3CB, PIK3R1, PIM1, PMS2, POLD1, POLE, PPARG, PPP2RIA, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSCIL1, WT1, XPO1, XRCC2, ZNF217, ZNF703, or any combination thereof.
40. The method of any of clauses 37 to 38, wherein the one or more gene loci comprise ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1β, IL-6, IL-6R, JAK1, JAK2, JAK3, KIT, KRAS, MEK, MET, MSI-H, mTOR, PARP, PD-1, PDGFR, PDGFRα, PDGFRB, PD-L1, PI3Kδ, PIGF, PTCH, RAF, RANKL, RET, ROS1, SLAMF7, VEGF, VEGFA, VEGFB, or any combination thereof.
41. The method of any one of clauses 1 to 40, further comprising generating, by the one or more processors, a report indicating the origin of the alteration in the sample.
42. The method of clause 41, further comprising transmitting the report to a healthcare provider.
43. The method of clause 42, wherein the report is transmitted via a computer network or a peer-to-peer connection.
44. A method for predicting an origin of an alteration in a sample from an individual, the method comprising:
receiving, using one or more processors, sequence read data associated with the sample;
selecting, using the one or more processors, a plurality of reads from the sequence read data based on the alteration;
determining, using the one or more processors, at least one feature characterizing the selected plurality of reads;
inputting, using the one or more processors, the at least one feature into a statistical model;
generating, using the one or more processors, a score indicative of the origin of the alteration by the statistical model; and
predicting, using the one or more processors, the origin of the alteration in the sample by comparing the score and one or more predefined thresholds.
45. The method of clause 44, wherein the alteration includes at least one of an insertion, a deletion, or a substitution.
46. The method of any of clauses 44 to 45, wherein the statistical model is a trained statistical model or an untrained statistical model.
47. The method of any of clauses 44 to 46, wherein the statistical model is a machine learning model.
48. The method of any of clauses 44 to 47, wherein the statistical model is a trained machine learning model and trained by:
receiving, using the one or more processors, training data including information quantifying features related to the alteration; and
training, using the one or more processors, the machine learning model based on the training data.
49. The method of any of clauses 44 to 48, wherein the score is indicative of a probability that the alteration is derived from a solid tumor.
50. The method of any of clauses 44 to 49, wherein the score is further indicative of a probability that the alteration is derived from clonal hematopoiesis.
51. The method of any of clauses 44 to 50, wherein the at least one feature comprises a fragment length for the selected plurality of reads, a start position of a fragment for the selected plurality of reads, an end position of a fragment for the selected plurality of reads, or a combination thereof.
52. The method of any of clauses 44 to 50, wherein the at least one feature comprises at least one fragmentomic characteristic of the sample.
53. The method of any of clause 52, wherein the at least one fragmentomic characteristic of the sample comprises at least one of: an amount of the selected plurality of reads having a specified length, a mean fragment length of the selected plurality of reads, a median fragment length of the selected plurality of reads, an interquartile range of fragment lengths of the selected plurality of reads, a distribution of fragment lengths of the selected plurality of reads, one or more peaks of fragment lengths for the selected plurality of reads, a fragment length for at least one of the selected plurality of reads, a fragment end motif, a start position of a fragment for the selected plurality of reads, and an end position of a fragment for the selected plurality of reads.
54. The method of clause 53, wherein a read of the selected plurality of reads is a cfDNA fragment.
55. The method of any of clauses 44 to 54, wherein the statistical model is configured to receive one or more additional features related to the alteration.
56. The method of clause 55, wherein the one or more additional features comprise an alteration depth, an allele frequency of the selected plurality of reads, an alteration coding type, a somatic determination, a germline determination, a zygosity determination, an age of the individual, a blood tumor mutational burden score, a protein level data, a gene level data, an odds ratio describing an enrichment of the alteration in older patients over younger patients adjusted by disease group, a p-value describing a significance of an enrichment of the alteration in older patients over younger patients adjusted by disease group, an odds ratio describing an enrichment for a specific amino acid change in older patients over younger patients adjusted by disease group, a p-value describing a significance of an enrichment for a specific amino acid change in older patients over younger patients adjusted by disease group, an odds ratio describing an enrichment of the alteration in a certain cancer type compared to other cancers, a p-value describing a significance of an enrichment of the alteration in a certain cancer type compared to other cancers, an odds ratio describing an enrichment for a specific amino acid change in a certain cancer type compared to other cancers, a p-value describing a significance of an enrichment for a specific amino acid change in a certain cancer type compared to other cancers, a tumor fraction, an inferred subclonal status, a distribution of variant level germline, somatic, subclonal somatic calls, variant allele frequency changes over time, a sample level mutational signature score, a description of pathogenicity measures, or any combination thereof.
57. The method of clause 56, wherein one or more of the somatic determination, the germline determination, and the zygosity determination is a computational determination.
58. The method of any of clauses 44 to 57, wherein the one or more predetermined thresholds comprise a first predetermined threshold, and wherein predicting the origin of the alteration in the sample by comparing the score and one or more predefined thresholds further comprises:
comparing, using the one or more processors, the score against the first predetermined threshold; and
in accordance with a determination that the score is greater than or equal to the first predetermined threshold, determining, using the one or more processors, that the alteration is not derived from a tumor.
59. The method of clause 58, wherein the one or more predetermined thresholds comprise a second predetermined threshold, and wherein predicting, using the one or more processors, the origin of the alteration in the sample by comparing the score and one or more predefined thresholds further comprises:
comparing, using the one or more processors, the score against the second predetermined threshold; and
in accordance with a determination that the score is less than or equal to the second predetermined threshold, determining, using the one or more processors, that the alteration is derived from the tumor.
60. The method of clause 59, wherein the second predetermined threshold is the same as the first predetermined threshold.
61. The method of clause 59, wherein the second predetermined threshold is different from the first predetermined threshold.
62. The method of clause 61, wherein predicting the origin of the alteration in the sample by comparing the score and one or more predefined thresholds further comprises:
in accordance with a determination that the score is less than the first predetermined threshold and that the score is greater than the second predetermined threshold, determining, using the one or more processors, that the score is inconclusive.
63. The method of any of clauses 44 to 62, wherein the one or more predetermined thresholds are determined by maximizing or minimizing one or more of a function of sensitivity and specificity and the area under a predictor's receiver operating characteristic curve.
64. The method of any of clauses 44 to 63, wherein the sample comprises a liquid biopsy sample and comprises cell-free DNA (cfDNA), circulating tumor DNA (ctDNA), or any combination thereof.
65. The method of any of clauses 44 to 64, further comprising: identifying, using the one or more processors, one or more of a treatment or a monitoring requirement for the individual based on the prediction.
66. The method of clause 65, wherein the monitoring requirement comprises:
obtaining, using the one or more processors, an additional sample from the individual; and
determining, using the one or more processors, an origin of the alteration in the additional sample using one or more tests.
67. The method of clause 66, wherein the one or more tests comprises at least one of a paired normal test or an orthogonal test.
68. The method of clause 67, wherein the paired normal test comprises sequencing peripheral blood mononuclear cells.
69. The method of clause 67, wherein the one or more tests comprises reflex testing of a tissue sample.
70. The method of any of clauses 44 to 69, further comprising: administering, using the one or more processors, one or more of a treatment or a monitoring type for the individual based on the prediction.
71. The method of clause 70, wherein the administering the monitoring type comprises: obtaining, using the one or more processors, an additional sample from the individual; and
determining, using the one or more processors, an origin of the alteration in the additional sample using one or more tests.
72. The method of any of clauses 44 to 71, further comprising: determining, using the one or more processors, an adequacy of the sample for clinical decision-making.
73. The method of clause 72, wherein the sample is determined to be inadequate for clinical decision-making if the alteration is not derived from a tumor.
74. The method of any of clauses 44 to 73, further comprising determining one or more biomarkers based on the score.
75. The method of clause 74, wherein the one or more biomarkers comprise at least one of: a tumor fraction or a blood tumor mutational burden (bTMB).
76. The method of any of clauses 44 to 75, further comprising obtaining training data, wherein the training data includes information quantifying features related to the alteration.
77. The method of clause 76, wherein the information quantifying features related to the alteration includes at least one of: fragmentomic characteristics for each of a plurality of training sequencing reads, additional features for the plurality of training sequencing reads, and origin information for the plurality of training sequence reads.
78. The method of clause 77, wherein the fragmentomic characteristics for each of a plurality of training sequencing reads comprises at least one of: an amount of a fragment having a specified length, a mean fragment length of the selected plurality of reads, a median fragment length of the selected plurality of reads, an interquartile range of the selected plurality of reads, a distribution of a fragment length of the selected plurality of reads, one or more peaks of a fragment length for the selected plurality of reads, a fragment length for the selected plurality of reads, a start position of a fragment for the selected plurality of reads, and an end position of a fragment for the selected plurality of reads.
79. The method of any of clauses 77 to 78, wherein the additional features for each of the plurality of training sequence reads comprise an alteration depth, an allele frequency of the selected plurality of reads, an alteration coding type, a somatic determination, a germline determination, a zygosity determination, an age of the individual, a blood tumor mutational burden score, a protein level data, a gene level data, or any combination thereof.
80. The method of any of clauses 76 to 79, wherein the training data is obtained by: obtaining, using the one or more processors, a matched sample pair comprising a white blood cell sample and a corresponding plasma sample;
receiving, using the one or more processors, white blood cell read data obtained from the white blood cell sample and plasma read data obtained from the plasma sample;
selecting, using the one or more processors, a plurality of training sequence reads from the white blood cell read data based on the alteration and further selecting a plurality of training sequence reads from the plasma read data based on the alteration;
determining, using the one or more processors, the information quantifying features related to the alteration from the plasma read data and white blood cell read data; and
determining, using the one or more processors, origin information based on a comparison of the selected plurality of training sequence reads from the plasma read data and the selected plurality of training sequence reads from white blood cell read data.
81. The method of clause 76 to 79, wherein the training data is obtained by:
receiving, using one or more processors, sequence read data obtained based on a training sample from a subject;
selecting, using the one or more processors, a plurality of training sequence reads from the sequence read data obtained based on the training sample based on a gene associated with the alteration;
classifying, using one or more processors, the plurality of training sequence reads into a first category corresponding to the alteration and a second category corresponding to a wild-type;
determining, using one or more processors, a plurality of first fragment length metrics for the first category;
determining, using one or more processors, a plurality of second fragment length metrics for the second category;
wherein the information quantifying features related to the alteration comprises the plurality of first fragment length metrics and the plurality of second fragment length metrics.
82. The method of clause 81, wherein the training further comprises:
inputting, using one or more processors, the information quantifying features related to the alteration into the statistical model;
obtaining, using one or more processors, a feature vector based on the first plurality of fragment length metrics and the second plurality of fragment length metrics;
determining, using one or more processors, a score based on the feature vector and one or more additional features; and
updating one or more weights associated with the statistical model based on the score.
83. The method of any of clauses 81 to 82, wherein a first fragment length metric of the plurality of first fragment length metrics comprises a plurality of first fragment amounts in the first category, each first fragment amount corresponding to a specified length for a plurality of lengths.
84. The method of any of clauses 81 to 83, wherein a second fragment length metric of the plurality of second fragment length metrics comprises a plurality of second fragment amounts in the second category, each second fragment amount having a specified length for the plurality of lengths.
85. The method of any of clauses 81 to 84, wherein the first fragment length metrics correspond to the alteration and the second fragment length metrics correspond to the wild-type.
86. The method of any of clauses 81 to 85, wherein the plurality of first fragment length metrics and the plurality of second fragment length metrics are stored in a two-dimensional array.
87. The method of any of clauses 81 to 86, wherein the feature vector includes one or more fragmentomic characteristics of the plurality of training sequence reads, one or more additional features of the plurality of training sequence reads, or a combination thereof.
88. The method of any of clauses 44 to 87, wherein the statistical model is part of a machine learning process.
89. The method of any of clauses 44 to 88, wherein the statistical model includes an artificial intelligence learning model.
90. The method of any of clauses 44 to 89, wherein the statistical model is at least one of a Bayesian model, a random forest model, a support vector machine learning model, a linear regression model, a non-linear regression model, a multivariate regression machine learning model, a robust machine learning model, a neural network model, a nearest neighbor machine learning model, a gradient boosting ensemble model, and a proportional hazards model.
91. The method of any of clauses 44 to 90, wherein the statistical model is a convolutional neural network (CNN) machine learning model.
92. The method of clause 91, wherein the CNN machine learning model comprises one or more one or more convolution layers, one or more pooling layers, one or more dropout layers, one or more dense layers, or a combination thereof.
93. The method of any of clauses 91 to 92, wherein the CNN machine learning model is configured to receive, an input, at least one fragmentomic characteristic of the sample and at least one additional feature.
94. The method of clause 93, further comprising:
processing the at least one fragmentomic characteristic of the sample with the CNN machine learning model;
concatenating an output of the CNN machine learning model with the one additional feature; and
inputting the concatenated output into a deep neural network, wherein generating the score indicative of the origin of the alteration is performed by the deep neural network.
95. The method of clause 93, wherein the CNN machine learning model is configured to extract one or more additional fragmentomic characteristics from the input.
96. The method of clause 93, wherein the at least one additional feature comprises an age of the individual, a blood tumor mutational burden score, a protein level data, a gene level data, or any combination thereof.
97. The method of any of clauses 44 to 96, wherein the statistical model is a supervised machine learning model or an unsupervised machine learning model.
98. The method of any of clauses 44 to 97, further comprising:
selecting, using the one or more processors, a plurality of reference reads from the sequence read data based on a location of a reference gene associated with the alteration;
determining, using the one or more processors, at least one feature characterizing the selected plurality of reference reads;
comparing, using the one or more processors, the at least one feature characterizing the selected plurality of reference reads and the at least one feature characterizing the selected plurality of reads to determine a reference score; and
inputting, using the one or more processors, the reference score into the statistical model.
99. The method of any of clauses 44 to 98, wherein the alteration is based on a predetermined user input.
100. The method of any of clauses 44 to 99, wherein the alteration is determined based on an algorithmic process.
101. The method of any of clauses 44 to 100, wherein the statistical model is configured to determine one or more fragmentomic characteristics based on the at least one feature characterizing the selected plurality of reads.
102. The method of any of clauses 44 to 101, wherein the sequence read data is obtained through the use of next-generation sequencing.
103. The method of any of clauses 44 to 102, wherein selecting a plurality of reads from the sequence read data based on the alteration comprises selecting sequencing reads from a genomic region where the alteration is located.
104. A method for predicting an origin of an alteration in a sample from an individual, the method comprising:
receiving, using one or more processors, sequence read data associated with the sample;
selecting, using the one or more processors, a plurality of reads from the sequence read data based on the alteration;
determining, using the one or more processors, at least one at least one fragmentomic characteristic based on the selected plurality of reads;
inputting, using the one or more processors, the at least one fragmentomic characteristic into a statistical model;
generating, using the one or more processors, a score indicative of the origin of the alteration by the statistical model; and
predicting, using the one or more processors, the origin of the alteration in the sample by comparing the score and one or more predefined thresholds.
105. The method of clause 104, wherein the alteration includes at least one of an insertion, a deletion, or a substitution.
106. The method of any of clauses 104 to 105, wherein the statistical model is a trained statistical model or an untrained statistical model.
107. The method of any of clauses 104 to 106, wherein the statistical model is a machine learning model.
108. The method of any of clauses 104 to 107, wherein the statistical model is a trained machine learning model and trained by:
receiving, using the one or more processors, training data including information quantifying features related to the alteration; and
training, using the one or more processors, the machine learning model based on the training data.
109. The method of any of clauses 104 to 108, wherein the score is indicative of a probability that the alteration is derived from a solid tumor.
110. The method of any of clauses 104 to 109, wherein the score is further indicative of a probability that the alteration is derived from clonal hematopoiesis.
111. The method of any of clauses 104 to 110, wherein the at least one feature comprises at least one fragmentomic characteristic of the sample.
112. The method of any of clause 111, wherein the at least one fragmentomic characteristic of the sample comprises at least one of: an amount of the selected plurality of reads having a specified length, a mean fragment length of the selected plurality of reads, a median fragment length of the selected plurality of reads, an interquartile range of fragment lengths of the selected plurality of reads, a distribution of fragment lengths of the selected plurality of reads, one or more peaks of fragment lengths for the selected plurality of reads, a fragment length for at least one of the selected plurality of reads, a fragment end motif, a start position of a fragment for the selected plurality of reads, and an end position of a fragment for the selected plurality of reads.
113. The method of clause 112, wherein a read of the selected plurality of reads is a cfDNA fragment.
114. The method of any of clauses 104 to 113, wherein the statistical model is configured to receive one or more additional features related to the alteration.
115. The method of clause 114, wherein the one or more additional features comprise an alteration depth, an allele frequency of the selected plurality of reads, an alteration coding type, a somatic determination, a germline determination, a zygosity determination, an age of the individual, a blood tumor mutational burden score, a protein level data, a gene level data, an odds ratio describing an enrichment of the alteration in older patients over younger patients adjusted by disease group, a p-value describing a significance of an enrichment of the alteration in older patients over younger patients adjusted by disease group, an odds ratio describing an enrichment for a specific amino acid change in older patients over younger patients adjusted by disease group, a p-value describing a significance of an enrichment for a specific amino acid change in older patients over younger patients adjusted by disease group, an odds ratio describing an enrichment of the alteration in a certain cancer type compared to other cancers, a p-value describing a significance of an enrichment of the alteration in a certain cancer type compared to other cancers, an odds ratio describing an enrichment for a specific amino acid change in a certain cancer type compared to other cancers, a p-value describing a significance of an enrichment for a specific amino acid change in a certain cancer type compared to other cancers, a tumor fraction, an inferred subclonal status, a distribution of variant level germline, somatic, subclonal somatic calls, variant allele frequency changes over time, a sample level mutational signature score, a description of pathogenicity measures, or any combination thereof.
116. The method of clause 115, wherein one or more of the somatic determination, the germline determination, and the zygosity determination is a computational determination.
117. The method of any of clauses 104 to 116, wherein the one or more predetermined thresholds comprise a first predetermined threshold, and wherein predicting the origin of the alteration in the sample by comparing the score and one or more predefined thresholds further comprises:
comparing, using the one or more processors, the score against the first predetermined threshold; and
in accordance with a determination that the score is greater than or equal to the first predetermined threshold, determining, using the one or more processors, that the alteration is not derived from a tumor.
118. The method of clause 117, wherein the one or more predetermined thresholds comprise a second predetermined threshold, and wherein predicting, using the one or more processors, the origin of the alteration in the sample by comparing the score and one or more predefined thresholds further comprises:
comparing, using the one or more processors, the score against the second predetermined threshold; and
in accordance with a determination that the score is less than or equal to the second predetermined threshold, determining, using the one or more processors, that the alteration is derived from the tumor.
119. The method of clause 118, wherein the second predetermined threshold is the same as the first predetermined threshold.
120. The method of clause 118, wherein the second predetermined threshold is different from the first predetermined threshold.
121. The method of clause 120, wherein predicting the origin of the alteration in the sample by comparing the score and one or more predefined thresholds further comprises:
in accordance with a determination that the score is less than the first predetermined threshold and that the score is greater than the second predetermined threshold, determining, using the one or more processors, that the score is inconclusive.
122. The method of any of clauses 104 to 121, wherein the one or more predetermined thresholds are determined by maximizing or minimizing one or more of a function of sensitivity and specificity and the area under a predictor's receiver operating characteristic (AUC).
123. The method of any of clauses 104 to 122, wherein the sample comprises a liquid biopsy sample and comprises cell-free DNA (cfDNA), circulating tumor DNA (ctDNA), or any combination thereof.
124. The method of any of clauses 104 to 123, further comprising: identifying, using the one or more processors, one or more of a treatment or a monitoring requirement for the individual based on the prediction.
125. The method of clause 124, wherein the monitoring requirement comprises:
obtaining, using the one or more processors, an additional sample from the individual; and
determining, using the one or more processors, an origin of the alteration in the additional sample using one or more tests.
126. The method of clause 125, wherein the one or more tests comprises at least one of a paired normal test or an orthogonal test.
127. The method of clause 126, wherein the paired normal test comprises sequencing peripheral blood mononuclear cells.
128. The method of clause 127, wherein the one or more tests comprises reflex testing of a tissue sample.
129. The method of any of clauses 104 to 128, further comprising: administering, using the one or more processors, one or more of a treatment or a monitoring type for the individual based on the prediction.
130. The method of clause 129, wherein the administering the monitoring type comprises:
obtaining, using the one or more processors, an additional sample from the individual; and
determining, using the one or more processors, an origin of the alteration in the additional sample using one or more tests.
131. The method of any of clauses 104 to 130, further comprising: determining, using the one or more processors, an adequacy of the sample for clinical decision-making.
132. The method of clause 131, wherein the sample is determined to be inadequate for clinical decision-making if the alteration is not derived from a tumor.
133. The method of any of clauses 104 to 132, further comprising determining one or more biomarkers based on the score.
134. The method of clause 133, wherein the one or more biomarkers comprise at least one of: a tumor fraction or a blood tumor mutational burden (bTMB).
135. The method of any of clauses 104 to 134, further comprising obtaining training data, wherein the training data includes information quantifying features related to the alteration.
136. The method of clause 135, wherein the information quantifying features related to the alteration includes at least one of: fragmentomic characteristics for each of a plurality of training sequencing reads, additional features for each of the plurality of training sequencing reads, and origin information for each of the plurality of training sequence reads.
137. The method of clause 136, wherein the fragmentomic characteristics for each of a plurality of training sequencing reads comprises at least one of: an amount of a fragment having a specified length, a mean fragment length of the selected plurality of reads, a median fragment length of the selected plurality of reads, an interquartile range of the selected plurality of reads, a distribution of a fragment length of the selected plurality of reads, one or more peaks of a fragment length for the selected plurality of reads, a fragment length for the selected plurality of reads, a start position of a fragment for the selected plurality of reads, and an end position of a fragment for the selected plurality of reads.
138. The method of any of clauses 136 to 137, wherein the additional features for each of the plurality of training sequence reads comprise an alteration depth, an allele frequency of the selected plurality of reads, an alteration coding type, a somatic determination, a germline determination, a zygosity determination, an age of the individual, a blood tumor mutational burden score, a protein level data, a gene level data, or any combination thereof.
139. The method of any of clauses 135 to 138, wherein the training data is obtained by: obtaining, using the one or more processors, a matched sample pair comprising a white blood cell sample and a corresponding plasma sample;
receiving, using the one or more processors, white blood cell read data obtained from the white blood cell sample and plasma read data obtained from the plasma sample;
selecting, using the one or more processors, a plurality of training sequence reads from the white blood cell read data based on the alteration and further selecting a plurality of training sequence reads from the plasma read data based on the alteration;
determining, using the one or more processors, the information quantifying features related to the alteration from the plasma read data and white blood cell read data; and
determining, using the one or more processors, origin information based on a comparison of the selected plurality of training sequence reads from the plasma read data and the selected plurality of training sequence reads from white blood cell read data.
140. The method of any of clauses 135 to 138, wherein the training data is obtained by:
receiving, using one or more processors, sequence read data obtained based on a training sample from a subject;
selecting, using the one or more processors, a plurality of training sequence reads from the sequence read data obtained based on the training sample based on a gene associated with the alteration;
classifying, using one or more processors, the plurality of training sequence reads into a first category corresponding to the alteration and a second category corresponding to a wild-type;
determining, using one or more processors, a plurality of first fragment length metrics for the first category;
determining, using one or more processors, a plurality of second fragment length metrics for the second category;
wherein the information quantifying features related to the alteration comprises the plurality of first fragment length metrics and the plurality of second fragment length metrics.
141. The method of clause 140, wherein the training further comprises:
inputting, using one or more processors, the information quantifying features related to the alteration into the statistical model;
obtaining, using one or more processors, a feature vector based on the first plurality of fragment length metrics and the second plurality of fragment length metrics;
determining, using one or more processors, a score based on the feature vector and one or more additional features; and
updating one or more weights associated with the statistical model based on the score.
142. The method of any of clauses 140 to 141, wherein a first fragment length metric of the plurality of first fragment length metrics comprises a plurality of first fragment amounts in the first category, each first fragment amount corresponding to a specified length for a plurality of lengths.
143. The method of any of clauses 140 to 142, wherein a second fragment length metric of the plurality of second fragment length metrics comprises a plurality of second fragment amounts in the second category, each second fragment amount having a specified length for the plurality of lengths.
144. The method of any of clauses 140 to 143, wherein the first fragment length metrics corresponds to the alteration and the second fragment length metrics corresponds to the wild-type.
145. The method of any of clauses 140 to 144, wherein the plurality of first fragment length metrics and the plurality of second fragment length metrics are stored in a two-dimensional array.
146. The method of any of clauses 140 to 145, wherein the feature vector includes one or more fragmentomic characteristics of the plurality of training sequence reads, one or more additional features of the plurality of training sequence reads, or a combination thereof.
147. The method of any of clauses 104 to 146, wherein the statistical model is part of a machine learning process.
148. The method of any of clauses 104 to 147, wherein the statistical model includes an artificial intelligence learning model.
149. The method of any of clauses 104 to 148, wherein the statistical model is at least one of a Bayesian model, a random forest model, a support vector model, a linear regression model, a non-linear regression model, a multivariate regression model, a robust model, a neural network model, a nearest neighbor model, a gradient boosting ensemble model, and a proportional hazards model.
150. The method of any of clauses 104 to 149, wherein the statistical model is a convolutional neural network (CNN) machine learning model.
151. The method of clause 150, wherein the CNN machine learning model comprises one or more one or more convolution layers, one or more pooling layers, one or more dropout layers, one or more dense layers, or a combination thereof.
152. The method of any of clauses 150 to 151, wherein the CNN machine learning model is configured to receive, an input, at least one fragmentomic characteristic of the sample and at least one additional feature.
153. The method of clause 152, further comprising:
processing the at least one fragmentomic characteristic of the sample with the CNN machine learning model;
concatenating an output of the CNN machine learning model with the one additional feature; and
inputting the concatenated output into a deep neural network,
wherein generating the score indicative of the origin of the alteration is performed by the deep neural network.
154. The method of any of clauses 152 to 153, wherein the CNN machine learning model is configured to extract one or more additional fragmentomic characteristics from the input.
155. The method of any of clauses 152 to 154, wherein the at least one additional feature comprises an age of the individual, a blood tumor mutational burden score, a protein level data, a gene level data, or any combination thereof.
156. The method of any of clauses 104 to 155, wherein the statistical model is a supervised machine learning model or an unsupervised machine learning model.
157. The method of any of clauses 104 to 156, further comprising:
selecting, using the one or more processors, a plurality of reference reads from the sequence read data based on a location of a reference gene associated with the alteration;
determining, using the one or more processors, at least one fragmentomic characteristic based on the selected plurality of reference reads;
comparing, using the one or more processors, the at least one fragmentomic characteristic based on the selected plurality of reference reads and the at least one fragmentomic characteristic based on the selected plurality of reads to determine a reference score; and
inputting, using the one or more processors, the reference score into the statistical model.
158. The method of any of clauses 104 to 157, wherein the alteration is based on a predetermined user input.
159. The method of any of clauses 104 to 158, wherein the alteration is determined based on an algorithmic process.
160. The method of any of clauses 104 to 159, wherein the sequence read data is obtained through the use of next-generation sequencing.
161. The method of any of clauses 104 to 160, wherein selecting a plurality of reads from the sequence read data based on the alteration comprises selecting sequencing reads from a genomic region where the alteration is located.
162. A method for diagnosing a disease, the method comprising: diagnosing that a subject has the disease based on a determination of an origin of an alteration in a sample from the subject, wherein the origin of the alteration is determined according to the method of any one of clauses 1 to 161.
163. A method of selecting an anti-cancer therapy, the method comprising: responsive to determining an origin of an alteration in a sample from a subject, selecting an anti-cancer therapy for the subject, wherein the origin of the alteration is determined according to the method of any one of clauses 1 to 161.
164. A method of treating a cancer in a subject, comprising: responsive to determining an origin of an alteration in a sample from the subject, administering an effective amount of an anti-cancer therapy to the subject, wherein the origin of the alteration is determined according to the method of any one of clauses 1 to 161.
165. A method for monitoring cancer progression or recurrence in a subject, the method comprising:
determining a first origin of an alteration in a first sample obtained from the subject at a first time point according to the method of any one of clauses 1 to 161;
determining a second origin of an alteration in a second sample obtained from the subject at a second time point; and comparing the first origin of the alteration to the second origin of the alteration, thereby monitoring the cancer progression or recurrence.
166. The method of clause 165, wherein the second origin of the alteration for the second sample is determined according to the method of any one of clauses 1 to 161.
167. The method of any of clauses 165 to 166, further comprising selecting an anti-cancer therapy for the subject in response to the cancer progression.
168. The method of any of clauses 165 to 166, further comprising administering an anti-cancer therapy to the subject in response to the cancer progression.
169. The method of any of clauses 165 to 166, further comprising adjusting an anti-cancer therapy for the subject in response to the cancer progression.
170. The method of any of clauses 165 to 169, further comprising adjusting a dosage of the anti-cancer therapy or selecting a different anti-cancer therapy in response to the cancer progression.
171. The method of clause 169, further comprising administering the adjusted anti-cancer therapy to the subject.
172. The method of any of clauses 165 to 171, wherein the first time point is before the subject has been administered an anti-cancer therapy, and wherein the second time point is after the subject has been administered the anti-cancer therapy.
173. The method of any of clauses 165 to 172, wherein the subject has a cancer, is at risk of having a cancer, is being routine tested for cancer, or is suspected of having a cancer.
174. The method of any of clauses 165 to 173, wherein the cancer is a solid tumor.
175. The method of any of clauses 165 to 174, wherein the cancer is a hematological cancer.
176. The method of any of clauses 165 to 175, wherein the anti-cancer therapy comprises chemotherapy, radiation therapy, immunotherapy, a targeted therapy, or surgery.
177. The method of any one of clauses 1 to 161, wherein the determination of the origin of the alteration in the sample is used in making suggested treatment decisions for the subject.
178. The method of any one of clauses 1 to 161, wherein the determination of the origin of the alteration in the sample is used in applying or administering a treatment to the subject.
179. A system comprising:
one or more processors; and
a memory communicatively coupled to the one or more processors and configured to store instructions that, when executed by the one or more processors, cause the system to:
   receive, using one or more processors, sequence read data associated with the sample;
   select, using the one or more processors, a plurality of reads from the sequence read data based on the alteration;
   determine, using the one or more processors, at least one feature characterizing the selected plurality of reads;
   input, using the one or more processors, the at least one feature into a statistical model;
   generate, using the one or more processors, a score indicative of the origin of the alteration by the statistical model; and
   predict, using the one or more processors, the origin of the alteration in the sample by comparing the score and one or more predefined thresholds.
180. The system of clause 179, wherein the alteration includes at least one of an insertion, a deletion, or a substitution.
181. The system of any of clauses 179 to 180, wherein the statistical model is a trained statistical model or an untrained statistical model.
182. The system of any of clauses 179 to 181, wherein the statistical model is a trained machine learning model and trained by:
receiving, using the one or more processors, training data including information quantifying features related to the alteration; and
training, using the one or more processors, the machine learning model based on the training data.
183. The system of any of clauses 179 to 182, wherein the score is indicative of a probability that the alteration is derived from a solid tumor.
184. The system of any of clauses 179 to 183, wherein the score is further indicative of a probability that the alteration is derived from clonal hematopoiesis.
185. The system of any of clauses 179 to 184, wherein the at least one feature comprises at least one fragmentomic characteristic of the sample.
186. The system of any of clause 185, wherein the at least one fragmentomic characteristic of the sample comprises at least one of: an amount of the selected plurality of reads having a specified length, a mean fragment length of the selected plurality of reads, a median fragment length of the selected plurality of reads, an interquartile range of fragment lengths of the selected plurality of reads, a distribution of fragment lengths of the selected plurality of reads, one or more peaks of fragment lengths for the selected plurality of reads, a fragment length for at least one of the selected plurality of reads, a fragment end motif, a start position of a fragment for the selected plurality of reads, and an end position of a fragment for the selected plurality of reads.
187. The system of clause 186, wherein a read of the selected plurality of reads is a cfDNA fragment.
188. The system of any of clauses 179 to 187, wherein the statistical model is configured to receive one or more additional features related to the alteration.
189. The system of clause 188, wherein the one or more additional features comprise an alteration depth, an allele frequency of the selected plurality of reads, an alteration coding type, a somatic determination, a germline determination, a zygosity determination, an age of the individual, a blood tumor mutational burden score, a protein level data, a gene level data, an odds ratio describing an enrichment of the alteration in older patients over younger patients adjusted by disease group, a p-value describing a significance of an enrichment of the alteration in older patients over younger patients adjusted by disease group, an odds ratio describing an enrichment for a specific amino acid change in older patients over younger patients adjusted by disease group, a p-value describing a significance of an enrichment for a specific amino acid change in older patients over younger patients adjusted by disease group, an odds ratio describing an enrichment of the alteration in a certain cancer type compared to other cancers, a p-value describing a significance of an enrichment of the alteration in a certain cancer type compared to other cancers, an odds ratio describing an enrichment for a specific amino acid change in a certain cancer type compared to other cancers, a p-value describing a significance of an enrichment for a specific amino acid change in a certain cancer type compared to other cancers, a tumor fraction, an inferred subclonal status, a distribution of variant level germline, somatic, subclonal somatic calls, variant allele frequency changes over time, a sample level mutational signature score, a description of pathogenicity measures, or any combination thereof.
190. The system of any of clauses 179 to 189, wherein the one or more predetermined thresholds comprise a first predetermined threshold, and wherein predicting the origin of the alteration in the sample by comparing the score and one or more predefined thresholds further comprises:
comparing, using the one or more processors, the score against the first predetermined threshold; and
in accordance with a determination that the score is greater than or equal to the first predetermined threshold, determining, using the one or more processors, that the alteration is not derived from a tumor.
191. The system of clause 190, wherein the one or more predetermined thresholds comprise a second predetermined threshold, and wherein predicting, using the one or more processors, the origin of the alteration in the sample by comparing the score and one or more predefined thresholds further comprises:
comparing, using the one or more processors, the score against the second predetermined threshold; and
in accordance with a determination that the score is less than or equal to the second predetermined threshold, determining, using the one or more processors, that the alteration is derived from the tumor.
192. The system of any of clauses 190 to 191, wherein the second predetermined threshold is the same as the first predetermined threshold.
193. The method of any of clauses 179 to 192, wherein the statistical model is part of a machine learning process.
194. The method of any of clauses 179 to 193, wherein the statistical model includes an artificial intelligence learning model.
195. The method of any of clauses 179 to 194, wherein the statistical model is at least one of a Bayesian model, a random forest model, a support vector machine learning model, a linear regression model, a non-linear regression model, a multivariate regression machine learning model, a robust machine learning model, a neural network model, a nearest neighbor machine learning model, a gradient boosting ensemble model, and a proportional hazards model.
196. The method of any of clauses 179 to 195, wherein the statistical model is a convolutional neural network (CNN) machine learning model.
197. The method of clause 196, wherein the CNN machine learning model comprises one or more one or more convolution layers, one or more pooling layers, one or more dropout layers, one or more dense layers, or a combination thereof.
198. A non-transitory computer-readable storage medium storing one or more programs, the one or more programs comprising instructions, which when executed by one or more processors of a system, cause the system to:
receive, using one or more processors, sequence read data associated with the sample;
select, using the one or more processors, a plurality of reads from the sequence read data based on the alteration;
determine, using the one or more processors, at least one feature characterizing the selected plurality of reads;
input, using the one or more processors, the at least one feature into a statistical model;
generate, using the one or more processors, a score indicative of the origin of the alteration by the statistical model; and
predict, using the one or more processors, the origin of the alteration in the sample by comparing the score and one or more predefined thresholds.
199. The non-transitory computer-readable storage medium of clause 198, wherein the alteration includes at least one of an insertion, a deletion, or a substitution.
200. The non-transitory computer-readable storage medium of any of clauses 198 to 199, wherein the statistical model is a trained statistical model or an untrained statistical model.
201. The non-transitory computer-readable storage medium of any of clauses 198 to 200, wherein the statistical model is a trained machine learning model and trained by:
receiving, using the one or more processors, training data including information quantifying features related to the alteration; and
training, using the one or more processors, the machine learning model based on the training data.
202. The non-transitory computer-readable storage medium of any of clauses 198 to 201, wherein the score is indicative of a probability that the alteration is derived from a solid tumor.
203. The non-transitory computer-readable storage medium of any of clauses 198 to 202, wherein the score is further indicative of a probability that the alteration is derived from clonal hematopoiesis.
204. The non-transitory computer-readable storage medium of any of clauses 198 to 203, wherein the at least one feature comprises at least one fragmentomic characteristic of the sample.
205. The non-transitory computer-readable storage medium of any of clause 204, wherein the at least one fragmentomic characteristic of the sample comprises at least one of: an amount of the selected plurality of reads having a specified length, a mean fragment length of the selected plurality of reads, a median fragment length of the selected plurality of reads, an interquartile range of fragment lengths of the selected plurality of reads, a distribution of fragment lengths of the selected plurality of reads, one or more peaks of fragment lengths for the selected plurality of reads, a fragment length for at least one of the selected plurality of reads, a fragment end motif, a start position of a fragment for the selected plurality of reads, and an end position of a fragment for the selected plurality of reads.
206. The non-transitory computer-readable storage medium of clause 205, wherein a read of the selected plurality of reads is a cfDNA fragment.
207. The non-transitory computer-readable storage medium of any of clauses 198 to 206, wherein the statistical model is configured to receive one or more additional features related to the alteration.
208. The non-transitory computer-readable storage medium of clause 207, wherein the one or more additional features comprise an alteration depth, an allele frequency of the selected plurality of reads, an alteration coding type, a somatic determination, a germline determination, a zygosity determination, an age of the individual, a blood tumor mutational burden score, a protein level data, a gene level data, an odds ratio describing an enrichment of the alteration in older patients over younger patients adjusted by disease group, a p-value describing a significance of an enrichment of the alteration in older patients over younger patients adjusted by disease group, an odds ratio describing an enrichment for a specific amino acid change in older patients over younger patients adjusted by disease group, a p-value describing a significance of an enrichment for a specific amino acid change in older patients over younger patients adjusted by disease group, an odds ratio describing an enrichment of the alteration in a certain cancer type compared to other cancers, a p-value describing a significance of an enrichment of the alteration in a certain cancer type compared to other cancers, an odds ratio describing an enrichment for a specific amino acid change in a certain cancer type compared to other cancers, a p-value describing a significance of an enrichment for a specific amino acid change in a certain cancer type compared to other cancers, a tumor fraction, an inferred subclonal status, a distribution of variant level germline, somatic, subclonal somatic calls, variant allele frequency changes over time, a sample level mutational signature score, a description of pathogenicity measures, or any combination thereof.
209. The non-transitory computer-readable storage medium of any of clauses 198 to 208, wherein the one or more predetermined thresholds comprise a first predetermined threshold, and wherein predicting the origin of the alteration in the sample by comparing the score and one or more predefined thresholds further comprises:
comparing, using the one or more processors, the score against the first predetermined threshold; and
in accordance with a determination that the score is greater than or equal to the first predetermined threshold, determining, using the one or more processors, that the alteration is not derived from a tumor.
210. The non-transitory computer-readable storage medium of clause 209, wherein the one or more predetermined thresholds comprise a second predetermined threshold, and wherein predicting, using the one or more processors, the origin of the alteration in the sample by comparing the score and one or more predefined thresholds further comprises:
comparing, using the one or more processors, the score against the second predetermined threshold; and
in accordance with a determination that the score is less than or equal to the second predetermined threshold, determining, using the one or more processors, that the alteration is derived from the tumor.
211. The non-transitory computer-readable storage medium of any of clauses 209 to 210, wherein the second predetermined threshold is the same as the first predetermined threshold.
212. The method of any of clauses 198 to 211, wherein the statistical model is part of a machine learning process.
213. The method of any of clauses 198 to 212, wherein the statistical model includes an artificial intelligence learning model.
214. The method of any of clauses 198 to 213, wherein the statistical model is at least one of a Bayesian model, a random forest model, a support vector machine learning model, a linear regression model, a non-linear regression model, a multivariate regression machine learning model, a robust machine learning model, a neural network model, a nearest neighbor machine learning model, a gradient boosting ensemble model, and a proportional hazards model.
215. The method of any of clauses 198 to 214, wherein the statistical model is a convolutional neural network (CNN) machine learning model.
216. The method of clause 215, wherein the CNN machine learning model comprises one or more one or more convolution layers, one or more pooling layers, one or more dropout layers, one or more dense layers, or a combination thereof.
217. A method for predicting an origin of an alteration in a sample from an individual, the method comprising:
receiving, using one or more processors, sequence read data associated with the sample;
selecting, using the one or more processors, a plurality of reads from the sequence read data based on the alteration;
determining, using the one or more processors, at least one feature characterizing the selected plurality of reads;
inputting, using the one or more processors, the at least one feature into a trained statistical model;
generating, using the one or more processors, a score indicative of the origin of the alteration by the trained statistical model; and
predicting, using the one or more processors, the origin of the alteration in the sample by comparing the score and one or more predefined thresholds.
218. A method for predicting an origin of an alteration in a sample from an individual, the method comprising:
receiving, using one or more processors, sequence read data associated with the sample;
selecting, using the one or more processors, a plurality of reads from the sequence read data based on the alteration;
determining, using the one or more processors, at least one feature characterizing the selected plurality of reads;
inputting, using the one or more processors, the at least one feature into a trained statistical model;
generating, using the one or more processors, a score indicative of the origin of the alteration by the trained statistical model, wherein the score is indicative of a probability that the alteration is derived from a tumor; and
predicting, using the one or more processors, the origin of the alteration in the sample by comparing the score and one or more predefined thresholds.

It should be understood from the foregoing that, while particular implementations of the disclosed methods and systems have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

1. A method comprising:
obtaining a first set of one or more samples from a subject;
isolating polynucleotides from the first set of one or more samples;
sequencing the isolated polynucleotides to produce sequence reads;
selecting a plurality of reads from the sequence read data based on an alteration in the first set of one or more samples;
determining at least one feature characterizing each of the selected plurality of reads;
inputting the at least one feature characterizing each of the selected plurality of reads into a trained machine learning model;
generating a score indicative of an origin of the alteration by the trained machine learning model; and
predicting the origin of the alteration in the sample by comparing the score and one or more predefined thresholds.

2.-44. (canceled)

45. The method of claim 1, wherein the alteration includes at least one of an insertion, a deletion, or a substitution.

46. The method of claim 1, wherein the statistical model is a trained statistical model, an untrained statistical model, or a machine learning model.

47. (canceled)

48. The method of claim 1, wherein the statistical model is a trained machine learning model and trained by:
receiving, using the one or more processors, training data including information quantifying features related to the alteration; and
training, using the one or more processors, the machine learning model based on the training data.

49. The method of claim 1, wherein the score is indicative of a probability that the alteration is derived from a solid tumor and/or wherein the score is further indicative of a probability that the alteration is derived from clonal hematopoiesis.

50. (canceled)

51. The method of claim 1, wherein the at least one feature comprises a fragment length for the selected plurality of reads, a start position of a fragment for the selected plurality of reads, an end position of a fragment for the selected plurality of reads, or a combination thereof.

52. The method of claim 1, wherein the at least one feature comprises at least one fragmentomic characteristic of the sample.

53.-57. (canceled)

58. The method of claim 1, wherein the one or more predetermined thresholds comprise a first predetermined threshold, and wherein predicting the origin of the alteration in the sample by comparing the score and one or more predefined thresholds further comprises:
comparing, using the one or more processors, the score against the first predetermined threshold; and
in accordance with a determination that the score is greater than or equal to the first predetermined threshold, determining, using the one or more processors, that the alteration is not derived from a tumor.

59.-62. (canceled)

63. The method of claim 1, wherein the one or more predetermined thresholds are determined by maximizing or minimizing one or more of a function of sensitivity and specificity and the area under a predictor's receiver operating characteristic curve.

64. The method of claim 1, wherein the sample comprises a liquid biopsy sample and comprises cell-free DNA (cfDNA), circulating tumor DNA (ctDNA), or any combination thereof.

65. The method of claim 1, further comprising: identifying, using the one or more processors, one or more of a treatment or a monitoring requirement for the individual based on the prediction.

66.-71. (canceled)

72. The method of claim 1, further comprising: determining, using the one or more processors, an adequacy of the sample for clinical decision-making.

73.-89. (canceled)

90. The method of claim 1, wherein the statistical model is at least one of a Bayesian model, a random forest model, a support vector machine learning model, a linear regression model, a non-linear regression model, a multivariate regression machine learning model, a robust machine learning model, a neural network model, a nearest neighbor machine learning model, a gradient boosting ensemble model, and a proportional hazards model.

91. The method of claim 1, wherein the statistical model is a convolutional neural network (CNN) machine learning model.

92.-97. (canceled)

98. The method of claim 1, further comprising:
selecting, using the one or more processors, a plurality of reference reads from the sequence read data based on a location of a reference gene associated with the alteration;
determining, using the one or more processors, at least one feature characterizing the selected plurality of reference reads;
comparing, using the one or more processors, the at least one feature characterizing the selected plurality of reference reads and the at least one feature characterizing the selected plurality of reads to determine a reference score; and
inputting, using the one or more processors, the reference score into the statistical model.

99. The method of claim 1, wherein the alteration is based on a predetermined user input or based on an algorithmic process.

100. (canceled)

101. The method of claim 1, wherein the statistical model is configured to determine one or more fragmentomic characteristics based on the at least one feature characterizing the selected plurality of reads.

102.-176. (canceled)

177. The method of claim 1, wherein the determination of the origin of the alteration in the sample is used in making suggested treatment decisions for the subject and/or is used in applying or administering a treatment to the subject.

178. (canceled)

179. A system comprising:
one or more processors; and
a memory communicatively coupled to the one or more processors and configured to store instructions that, when executed by the one or more processors, cause the system to:
receive, using one or more processors, sequence read data associated with the sample;
select, using the one or more processors, a plurality of reads from the sequence read data based on the alteration;
determine, using the one or more processors, at least one feature characterizing the selected plurality of reads;
input, using the one or more processors, the at least one feature into a statistical model;
generate, using the one or more processors, a score indicative of the origin of the alteration by the statistical model; and
predict, using the one or more processors, the origin of the alteration in the sample by comparing the score and one or more predefined thresholds.

180.-197. (canceled)

198. A non-transitory computer-readable storage medium storing one or more programs, the one or more programs comprising instructions, which when executed by one or more processors of a system, cause the system to:
receive, using one or more processors, sequence read data associated with the sample;
select, using the one or more processors, a plurality of reads from the sequence read data based on the alteration;
determine, using the one or more processors, at least one feature characterizing the selected plurality of reads;
input, using the one or more processors, the at least one feature into a statistical model;
generate, using the one or more processors, a score indicative of the origin of the alteration by the statistical model; and
predict, using the one or more processors, the origin of the alteration in the sample by comparing the score and one or more predefined thresholds.

199.-218. (canceled)
